(19) United States
(12) Patent Application Publication
(10) Pub. No.: US 2025/0109207 A1
(43) Pub. Date: Apr. 3, 2025

(54) CHIMERIC HEAVY CHAIN CONSTANT DOMAINS WITH REDUCED BINDING TO FC GAMMA RECEPTORS AND USES THEREOF

(71) Applicant: Regeneron Pharmaceuticals, Inc., Tarrytown, NY (US)

(72) Inventors: Naga Suhasini AVVARU, Briarcliff Manor, NY (US); Samuel DAVIS, New York, NY (US); Chia-Yang LIN, Scarsdale, NY (US); Yang SHEN, Scarsdale, NY (US)

(73) Assignee: Regeneron Pharmaceuticals, Inc., Tarrytown, NY (US)

(21) Appl. No.: 18/857,898

(22) PCT Filed: Apr. 20, 2023
(86) PCT No.: PCT/US2023/066036

§ 371 Date: Oct. 18, 2024

Related U.S. Application Data
(60) Provisional application No. 63/333,293, filed on Apr. 21, 2022.

(51) Int. Cl. C07K 16/28; C07K 16/40

(52) U.S. Cl. CPC C07K 16/2863; C07K 16/40; C07K 2317/31; C07K 2317/35; C07K 2317/52; C07K 2317/524; C07K 2317/526; C07K 2317/53; C07K 2317/55; C07K 2317/622; C07K 2317/732; C07K 2317/92

(57) ABSTRACT

The present disclosure relates to chimeric heavy chain constant domains having reduced effector function. Also disclosed are recombinant polypeptides comprising such chimeric heavy chain constant domains, including antibodies such as multispecific antibodies, fusion proteins, and other recombinant proteins. Nucleic acids encoding such recombinant polypeptides are also disclosed, as well as cells expressing such recombinant polypeptides and pharmaceutical compositions comprising such recombinant polypeptides.

### 1. CROSS REFERENCE TO RELATED APPLICATIONS

This application claims the priority benefit of U.S. provisional application No. 63/333,293, filed on Apr. 21, 2022, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Apr. 12, 2023, is named RGN-013WO_SL.xml and is 58,069 bytes in size.

### 3. BACKGROUND

Antibodies of the Ig class are attractive therapeutic agents. IgGs exist as four subclasses in human: IgG1, IgG2, IgG3, and IgG4. The heavy chain constant (CH) region of IgG comprises three domains, CH1, CH2, CH3, and a hinge linking CH1 and CH2. Although the role of each subclass appears to vary between species, the heavy chain constant domain is responsible for various biological effector functions. The human IgG subclasses mediate several cellular immune responses through their interaction with Fcγ (FcγRs), such as cell killing, phagocytosis and opsonization. Such interaction involves binding of at least functional CH2 and CH3 domains of a heavy chain constant region to an FcγR on the surface of effector cells, such as natural killer cells and activated macrophages. Complement-mediated lysis can also be triggered by the interaction of the Fc region (e.g., CH2 and CH3 domains) with various complement components.

Effector functions are useful in some antibody therapies, such as treatment of some cancers or pathogens, in which effector function is primarily or at least partially responsible for killing cancer cells or the pathogen. However, other antibody therapies are mediated entirely or predominantly by effector-independent mechanisms, such as inhibiting a receptor-ligand interaction or agonizing a receptor. In such therapies, antibody effector functions serve little or no useful purpose but can result in undesired effects, including inflammation. In such circumstances, it may be advantageous to engineer the Fc receptor binding properties of an antibody so as to inhibit some or all of the available effector mechanisms, without substantially affecting the antibody's pharmacokinetic properties, immunogenicity and variable regions specificity and affinity. Several groups have undertaken such antibody engineering.

IgG heavy chain constant regions have been mutated in various positions to test the effect of amino acids on IgG/FcγR interaction (see, e.g., Canfield and Morrison, 1991, J Exp Med 73: 1483-1491; Chappel et al., 1993, JSC 268(33):25124-31; and Armour et al., Eur J Immunol 29:2613-24). Several amino acid residues in the hinge region and in the CH2 domain of a heavy chain constant region have been proposed as mediating binding to Fcγ receptors (see Sarmay et al., 1992, Mol Immunol 29:633-9; Greenwood et al., Eur J Immunol 23(5):1098, Morgan et al., 1995, Immunology 86:319; Stevenson, 1997, Chem Immunol 65:57-72). Glycosylation of a site (N297) in the CH2 domain and variations in the composition of its carbohydrates also strongly affect the IgG/FcγR interaction (Stevenson, 1997, Chem Immunol 65:57-72; Siberil et al. 2006, Immunol Ltrs 106:111-118). WO2014121087 describes a chimeric IgG comprising IgG1 upper hinge, IgG1 lower hinge, IgG4 CH2, and IgG1 CH3, in which amino acids in the lower hinge region are replaced with corresponding amino acids from the human IgG2 isotype so as to reduce FcγR binding without unacceptable conformational changes and consequent immunogenicity. WO2016161010A2 describes engineered IgG heavy chain constant domains having a modified hinge region in which amino acid positions 233-236 (EU numbering) are modified to Gly, Gly, Gly and unoccupied; Gly, Gly, unoccupied, and unoccupied; Gly, unoccupied, unoccupied, and unoccupied; or all unoccupied, with positions numbered by EU numbering (as shown in FIG. 1 of WO2016161010A2).

Having alternatives to those chimeric heavy chain constant domains known in the art is valuable. Having alternative chimeric heavy chain constant regions based on different IgG subclasses allows, e.g., for antibody activity optimization, and improved expression and production. Accordingly, heavy chain constant regions with reduced binding to FcγRs, and therapeutic polypeptides (e.g., recombinant antibodies) comprising the same, are sought that can be used to treat diseases or conditions in which effector function is to be minimized.

### 4. SUMMARY

The present disclosure relates to chimeric heavy chain constant domains with reduced Fc receptor and/or effector function.

Chimeric heavy chain constant domains of the disclosure are based on an IgG1 heavy chain constant domain in which the hinge region is modified. Unexpectedly, incorporating the chimeric constant domains of the disclosure into exemplary antibodies improved their expression and activity in addition to reduction of Fc receptor and effector function (see, e.g., Section 8.2 (Example 1), Section 8.5 (Example 4), Section 8.6 (Example 5) and Section 8.7 (Example 6)).

The present disclosure thus provides recombinant proteins, e.g., fusion proteins, comprising chimeric heavy chain constant domains of the disclosure. The chimeric heavy chain constant domains can advantageously be used as dimerization moieties in a variety of engineered proteins, e.g., recombinant antibodies, immunocytokines, and soluble receptors. In addition to a chimeric heavy chain constant domain of the disclosure, a recombinant polypeptide can include one or more target binding domains (e.g., one or more Fab moieties, one or more scFv moieties, or a combination thereof) and/or one or more linker moieties separating one or more moieties in the recombinant protein. In some embodiments, the recombinant protein is a recombinant antibody, for example a recombinant multispecific antibody.

Exemplary chimeric heavy chain constant domains are disclosed in Section 6.2.1. Exemplary recombinant proteins comprising the constant domains are disclosed in Section 6.2, Group A numbered embodiments 1 to 105 and 110 to 214, Group C numbered embodiments 1 to 45, and Group D numbered embodiments 1 to 24. Where the recombinant protein is an antibody, exemplary target binding domains for incorporation in the antibody are disclosed in Section 6.2.2 and exemplary linkers useful for connecting the constant domains to the target binding domains or different components of the target binding domains are described in Section 6.2.5.

The disclosure further provides nucleic acids encoding the recombinant proteins of the disclosure. The nucleic acids can be in the form of a single nucleic acid (e.g., a vector encoding two or more polypeptide chains) or a plurality of nucleic acids (e.g., two or more vectors encoding different polypeptide chains). The disclosure further provides host cells and cell lines engineered to express the nucleic acids and recombinant proteins of the disclosure. Exemplary nucleic acids, host cells, and cell lines, are described in Section 6.3, Group A numbered embodiments 108, 109, 216 and 217, Group C numbered embodiments 48 to 51, and Group D numbered embodiments 25 to 27.

Methods of producing the recombinant polypeptides and methods of using the constant domains of the disclosure to increase expression and/or activity of a recombinant protein are described in Section 6.3, Group A numbered embodiments 218 to 219, Group B numbered embodiments 1 to 69, Group C numbered embodiment 52, and Group D numbered embodiments 28 to 41. In certain embodiments, increased expression of a recombinant protein of the disclosure is evidenced by increased protein yield and/or production, for example as increased total amount of protein obtained from an expression system, such as an expression system described in Section 8.1.6. In certain embodiments, increased activity of a recombinant protein of the disclosure is evidenced by increased target binding and/or signal modulation.

The disclosure further provides compositions, e.g., populations of proteins and pharmaceutical compositions comprising the recombinant proteins of the disclosure. Exemplary compositions are described in Section 6.4, Group A numbered embodiments 106, 107, 214 and 215, Group B numbered embodiments 70 to 72, Group C numbered embodiments 46, 47 and 53 to 55 and Group D numbered embodiments 42 to 46.

### 5. BRIEF DESCRIPTION OF THE FIGURES

FIG. 1 depicts the wild type sequence of the heavy chain constant region of human IgG1 (human IGHG1 heavy chain constant region; UniProt Accession No. P01857). CH1=amino acids 1-98: upper hinge=amino acids 99-108: core hinge=109-112; lower hinge=113-121; CH2=120-223; CH3=224-330. Indicated amino acid numbering is relative to the depicted sequence. The upper, core, and lower hinge regions are boxed. As shown in the figure, the last two amino acids of the lower hinge correspond to the first two amino acids of the CH2 domain. FIG. 1 discloses SEQ ID NO: 48.

FIG. 2 depicts the wild type sequence of the heavy chain constant region of human IgG2 (human IGHG2 heavy chain constant region; UniProt Accession No. P01859). CH1=amino acids 1-98; upper hinge=amino acids 99-105; core hinge=106-109; lower hinge=110-117; CH2=116-219; CH3=220-326. Indicated amino acid numbering is relative to the depicted sequence. As shown in the figure, the last two amino acids of the lower hinge correspond to the first two amino acids of the CH2 domain. FIG. 2 discloses SEQ ID NO: 49.

FIG. 3 depicts the wild type sequence of the heavy chain constant region of human IgG4 (human IGHG4 heavy chain constant region; UniProt Accession No. P01861). CH1=amino acids 1-98; upper hinge=amino acids 99-105; core hinge=106-109; lower hinge=110-118; CH2=117-220; CH3=221-227. Indicated amino acid numbering is relative to the depicted sequence. As shown in the figure, the last two amino acids of the lower hinge correspond to the first two amino acids of the CH2 domain. FIG. 3 discloses SEQ ID NO: 50.

FIG. 4 depicts an amino acid sequence alignment of the upper hinge, core hinge, lower hinge, CH2, and CH3 of the noted chimeric IgG heavy chain constant domain constructs. Indicated amino acid numbering is EU numbering. Shaded cells of the lower hinge indicate amino acids also corresponding to the first two amino acids of the CH2 domain. FIG. 4 discloses SEQ ID NO:51 (rows 3 and 4), SEQ ID NO:52 (row 5), SEQ ID NO:53 (row 6), SEQ ID NO:54 (row 7), and SEQ ID NO:55 (row 8).

FIG. 5 depicts representative data demonstrating antibody titers of the depicted alternative format antibodies comprising hetero-dimers of either IgG4 S108P/IgG4 S108P Star (H315R, Y316F) or IgG1 PVA/IgG1 PVA Star (H315R, Y316F), and having the depicted 2+1 N-scFv format, following stable expression in Chinese hamster ovary (CHO) cells. Linkers of varying lengths between the Fab and scFv were tested.

FIGS. GA-GG depict representative enzyme-linked immunosorbent assay (ELISA) data demonstrating binding of the noted controls and antibodies to hFCRγ1 (FIG. 6A); hFCRγ2A (H131) (FIG. 6B); hFCRγ2A (R131) (FIG. 6C); hFCRγ2B (FIG. 60); hFCRγ3A (V158) (FIG. 6E); hFCRγ3A (F158) (FIG. 6F); and hFCRγ3B (FIG. 7G). Descriptions of the control and test antibodies are provided in Table 3.

FIG. 7 depicts representative results from a surrogate antibody dependent cell-mediated cytotoxicity (ADCC) assay in which the indicated 2+1 N-scFv alternate format antibodies (AF1) with differing Fc regions were tested, along with controls.

FIG. 8 depicts representative results from a surrogate ADCC assay in which the indicated 2+1 N-Fab alternate format antibodies (AF2) with differing Fc regions were tested, along with controls.

FIG. 9 depicts representative results from a luciferase reporter assay demonstrating 2+1 N-scFv format antibodies (AF1) with differing Fc regions along with controls caused activation of HEK293.SREluc.hFGFR1c.hKLB cells.

FIG. 10 depicts representative results from a luciferase reporter assay demonstrating 2+1 N-Fab format antibodies (AF2 and AF3) with differing Fc regions along with controls caused activation of HEK293.SREIuc.hFGFR1c.hKLB cells.

FIG. 11 depicts representative results from phospho-ERK activation assay demonstrating 2+1 N-scFv (AF1) or 2+1 N-Fab (AF3) format antibodies with either with IgG4 S108P or IgG1 PVA Fc regions or His.hFGF21 caused activation in primary human adipocytes.

FIG. 12 depicts representative flow binding assay results demonstrating that bispecific antibodies with IgG1 PVA Fc regions bind to the cell surface target with higher max MFI signal than bispecific antibodies with IgG4 S108P Fc regions.

FIGS. 13A-13E depict negative stain EM 2D class averages of IgG-CD40 complexes. FIG. 13A is an exemplary negative stain EM 2D image, displaying features corresponding to Fc, Fab, and CD40, as well as Fab-Fab angle. FIG. 13B is a diagram of FIG. 13A with its components. FIG. 130C displays the 2D class averages of IgG1-CD40 complex, FIG. 13D displays the 2D class averages of IgG1-PVA-CD40 complex, and FIG. 13E displays the 2D class averages of IgG2-CD40 complex.

### 6. DETAILED DESCRIPTION

### 6.1. Definitions

About. Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And and Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected), In some places in the text, the term “and/or” is used for the same purpose, which shall not be construed to imply that “or” is used with reference to mutually exclusive alternatives.

Antibody: The term “antibody” as used herein includes any form of antibody with a least one antigen binding fragment, including monovalent fragments (e.g., an scFv), bivalent tetrameric molecules of two heavy chains and two light chains, and higher order complexes of any of these. An antibody can be mono-specific, in which case all binding regions have the same specificity, or multi-specific in which the binding sites have at least two specificities (e.g., bispecific). The term “antibody” encompasses monoclonal antibodies, humanized antibodies, human antibodies, chimeric antibodies, and the like.

Associated: The term “associated” in the context of a chimeric constant domain, a recombinant polypeptide comprising a chimeric constant domain, or a component of the recombinant polypeptide (e.g., an antigen-binding domain) refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce, e.g., a functional antigen-binding domain or an Fc region. Examples of associations that might be present within a recombinant polypeptide of the disclosure or between a recombinant polypeptide of the disclosure and one or more additional polypeptides include (but are not limited to) associations between homodimeric or heterodimeric chimeric heavy chain constant domains of the disclosure, associations between VH and VL regions in a Fab or scFv, associations between CH1 and CL in a Fab, and associations between CH3 and OH3 in a domain substituted Fab.

Bivalent: The term “bivalent” as used herein in reference to an antibody comprising a recombinant polypeptide and/or chimeric constant domain of the disclosure means that the antibody has two antigen-binding moieties (e.g., two antigen binding fragments of an antibody, or a first antigen binding fragment of a first antibody and a second antigen binding fragment of a second antibody). An antibody comprising a recombinant polypeptide and/or chimeric constant domain of the disclosure may be bivalent for one type of moiety (e.g., two antigen binding fragments of a first antibody) and monovalent for another type of moiety (e.g., a single antigen binding fragment of a second antibody).

Chimeric Heavy Chain Constant Domain and Chimeric Constant Domain: The terms “chimeric heavy chain constant domain” and “chimeric constant domain”, when used in relation to the constant domain of the disclosure, are used interchangeably to refer to an IgG1 constant domain comprising an IgG1 upper hinge domain, an IgG1 core hinge domain, and an IgG1 lower hinge domain having a substitution/deletion mutation ELLG (SEQ ID NO: 23)→PVA- (or “P-V-A-absent”) at amino acid positions 233-236 (EU numbering), an IgG1 CH2 domain, and an IgG1 CH3 domain. In some embodiments, a chimeric constant domain also comprises an IgG1 CH1 domain. In some embodiments a chimeric constant domain can be further modified to, e.g., further alter effector function and/or provide for heterodimerization. Chimeric constant domains of the disclosure are further described in Section 6.2.

The chimeric constant domain is capable of facilitating an association between two recombinant polypeptide chains to form a dimer. The two chimeric constant domains in the dimer can be identical, or can be different. The resulting dimer can thus be a homodimer or a heterodimer.

Chimeric constant domains comprising an IgG1 lower hinge domain having a substitution/deletion mutation ELLG (SEQ ID NO: 23)→PVA- (or “P-V-A-absent”) are sometimes referred to as having an “IgG1 PVA” isotype or similar terms.

Dimerization Moiety: The term “dimerization moiety” refers to a polypeptide chain or an amino acid sequence capable of facilitating an association between two polypeptide chains to form a dimer. A first dimerization moiety can associate with an identical second dimerization moiety, or can associate with a second dimerization moiety that is different from the first. In some embodiments, a dimerization moiety is a recombinant constant domain of the disclosure, with the association of two recombinant constant domains to form an Fc region. Thus, the Fc region can be homodimeric or heterodimeric.

EC50: The term “EC50” refers to the half maximal effective concentration of a molecule, such as an antibody comprising a recombinant polypeptide and/or a chimeric constant domain of the disclosure, which induces a response halfway between the baseline and maximum after a specified exposure time. The EC50 essentially represents the concentration of an antibody where 50% of its maximal effect is observed. In certain embodiments, the EC50 value equals the concentration of an antibody that gives half-maximal activation in a luciferase reporter assay.

Fab: The term “Fab” refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of an immunoglobulin heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations. A chimeric constant domain of the disclosure comprises an Fc domain.

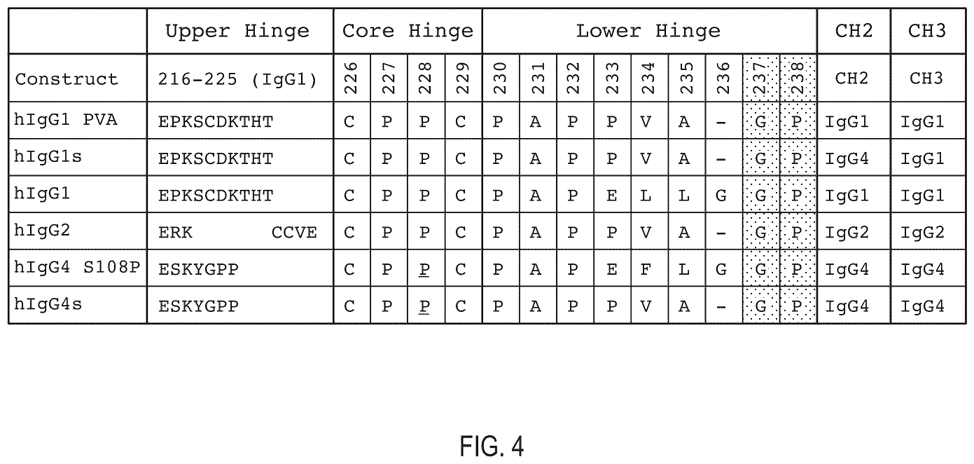

Hinge Region or Hinge Domain: The terms “hinge region” and “hinge domain” as used herein refer to consecutive amino acid residues that connect the C-terminus of the CH1 domain to the N-terminus of the CH2 domain of an immunoglobulin. In human IgG1, IgG2 and IgG4, the hinge region runs from residue 216 to 238 by EU numbering, with residues 237 and 238 overlapping with the CH2 domain. Residues 216 to 225 (EU numbering) form an upper hinge, residues 226 to 229 (EU numbering) form a middle (or core) hinge, and residues 230-238 (EU numbering) form a lower hinge. The upper and middle hinges of IgG1, IgG2 and IgG4 are 12-15 consecutive amino acids encoded by a distinct hinge exon. The lower hinge includes several N-terminal amino acids of the CH2 domain (encoded by the CH2 exon) (Brekke et al., 1995, Immunology Today 16(2):85-90). See, e.g., FIG. 4.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a recombinant polypeptide of the disclosure, a host cell can be a cell line of mammalian origin or mammalian-like characteristics, such as monkey kidney cells (COS, e.g., COS-1, COS-7), HEK293 (and derivatives such as Expi293 which have been adapted for higher density growth), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Monovalent: The term “monovalent” as used herein in reference to an antibody comprising a recombinant polypeptide and/or chimeric constant domain of the disclosure means that the antibody has one antigen-binding moiety specific for a first target molecule. The antibody comprising a recombinant polypeptide and/or chimeric constant domain of the disclosure may be monovalent for one type of moiety (e.g., a single antigen binding fragment of a first antibody) and bivalent for another type of moiety (e.g., two antigen binding fragments of a second antibody).

Multivalent: The term “multivalent” as used herein in reference to an antibody comprising a recombinant polypeptide and/or chimeric constant domain of the disclosure means that the antibody has two or more antigen-binding moieties (e.g., two antigen binding fragments of an antibody, or a first antigen binding fragment of a first antibody and a second antigen binding fragment of a second antibody). An antibody comprising a recombinant polypeptide and/or chimeric constant domain of the disclosure may be multivalent for one type of moiety (e.g., two or more antigen binding fragments of a first antibody) and monovalent for another type of moiety (e.g., a single antigen binding fragment of a second antibody).

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Recombinant Polypeptide: The term “recombinant polypeptide” refers to a polypeptide comprising a chimeric constant domain of the disclosure. Generally, a recombinant polypeptide comprises a chimeric constant domain of the disclosure and at least one antigen-binding moiety.

In the context of the disclosure, the term “recombinant polypeptide” sometimes refers to the core component(s) of the molecule, namely the chimeric constant domain and sometimes also an antigen-binding moiety. It is to be understood that the term “recombinant polypeptide” extends also to polypeptides comprising additional features, e.g., one or more stabilization moieties, one or more linker moieties, and any combination of the foregoing, unless the context dictates otherwise.

Single Chain Fv or scFv: The term “single chain Fv” or “scFv” as used herein refers to a polypeptide chain comprising the VH and VL domains of antibody, where these domains are present in a single polypeptide chain.

Specifically (or selectively) binds: The term “specifically (or selectively) binds” as used herein means that an antigen-binding moiety, e.g., an antibody, or antigen binding domain (“ABD”) thereof, forms a complex with a target molecule that is relatively stable under physiologic conditions. Specific binding can be characterized by a K_{D} of about 5×10⁻²M or less (e.g., less than 5×10²M, less than 10⁻²M, less than 5×10³M, less than 10³M, less than 5×10⁻⁴M, less than 10⁻⁴M, less than 5×10⁻⁵ M, less than 10⁻⁵ M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment to a target molecule are well known in the art and include, for example, equilibrium dialysis, surface plasmon resonance (e.g., Biacore assays), fluorescent-activated cell sorting (FACS) binding assays and the like. An antigen-binding moiety that specifically binds a target molecule from one species can, however, have cross-reactivity to the target molecule from one or more other species.

Target Binding Domain: The term “target binding domain” as used herein refers to a polypeptide sequence or group of associate polypeptide sequences capable of specific, non-covalent, and reversible binding to a target molecule. The term includes “antigen binding domains” and “antigen binding fragments”, which refer to the portion of an antibody that is capable of specific, non-covalent, and reversible binding to a target molecule. “Target binding domain” also encompasses the target binding portion of a receptor (e.g., a TNF receptor) that is capable of specific, non-covalent, and reversible binding to a target molecule (e.g., TNF). Target binding domains can be incorporated into recombinant polypeptides of the disclosure. In some embodiments, recombinant polypeptides comprising one or more target binding domains can be included in an antibody or a fusion protein. Such antibodies are described in Section 6.2.2, infra. Fusion proteins are described in Section 6.2.3, infra.

Target Molecule: The term “target molecule” as used herein refers to any biological molecule (e.g., protein, carbohydrate, lipid, or combination thereof) expressed on a cell surface or in the extracellular matrix that can be specifically bound by an antigen-binding moiety in an antibody comprising recombinant polypeptides of the disclosure.

Universal Light Chain: The term “universal light chain” as used herein in the context of an antigen-binding moiety refers to a light chain polypeptide capable of pairing with the heavy chain region of the antigen-binding moiety and also capable of pairing with other heavy chain regions. Universal light chains are also known as “common light chains.”

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 6.2. Recombinant Polypeptides

The present disclosure provides recombinant polypeptides comprising chimeric constant domains based on the IgG1 heavy chain constant domain in which the hinge region is modified to reduce Fc receptor and/or effector function. The recombinant polypeptides of the disclosure can function as dimerization moieties, capable of facilitating an association between two polypeptide chains to form a dimer. In some embodiments, recombinant polypeptides of the disclosure can dimerize to form an antibody. Antibodies comprising recombinant polypeptides of the disclosure are described in Section 6.2.2, infra. In other embodiments, recombinant polypeptides of the disclosure can dimerize to form a fusion protein. Fusion proteins comprising recombinant polypeptides of the disclosure are described in Section 6.2.3, infra. Recombinant polypeptides of the disclosure included in an antibody or fusion protein of the disclosure can comprise, in addition to a chimeric constant domain of the disclosure, one or more target binding domains (e.g., one or more Fab moieties, one or more scFv moieties, one or more target receptors or binding fragments thereof) and/or one or more linker moieties separating one or more moieties in the recombinant polypeptide. Target binding domains are described in Section 6.2.4. Linkers useful in recombinant polypeptides of the disclosure are described in Section 6.2.5.

### 6.2.1. Chimeric Constant Domains

The present disclosure provides chimeric constant domains based on the IgG1 heavy chain constant domain. Chimeric constant domains of the disclosure comprise an IgG1 upper hinge domain, an IgG1 lower hinge domain having the substitution/deletion mutation ELLG (SEQ ID NO: 23)→PVA- at amino acid positions 233-236 (EU numbering), an IgG1 CH2 domain, and an IgG1 CH3 domain. In some embodiments, a chimeric constant domain also comprises an IgG1 CH1 domain or a fragment thereof.

IgG heavy chain constant regions have been mutated in various positions to test the effect of amino acids on IgG/FcγR interaction (see, e.g., Canfield and Morrison, 1991, J Exp Med 73: 1483-1491; Chappel et al., 1993, JSC 268(33):25124-31; and Armour et al., Eur J Immunol 29:2613-24). Several amino acid residues in the hinge region and in the CH2 domain of a heavy chain constant region have been proposed as mediating binding to Fcγ receptors (see Sarmay et al., 1992, Mol Immunol 29:633-9: Greenwood et al., Eur J Immunol 23(5):1098, Morgan et al., 1995, Immunology 86:319; Stevenson, 1997, Chem Immunol 65:57-72). Glycosylation of a site (N297) in the CH2 domain and variations in the composition of its carbohydrates also strongly affect the IgG/FcγR interaction (Stevenson, 1997, Chem Immunol 65:57-72; Siberil et al., 2006, Immunol Ltrs 106:111-118).

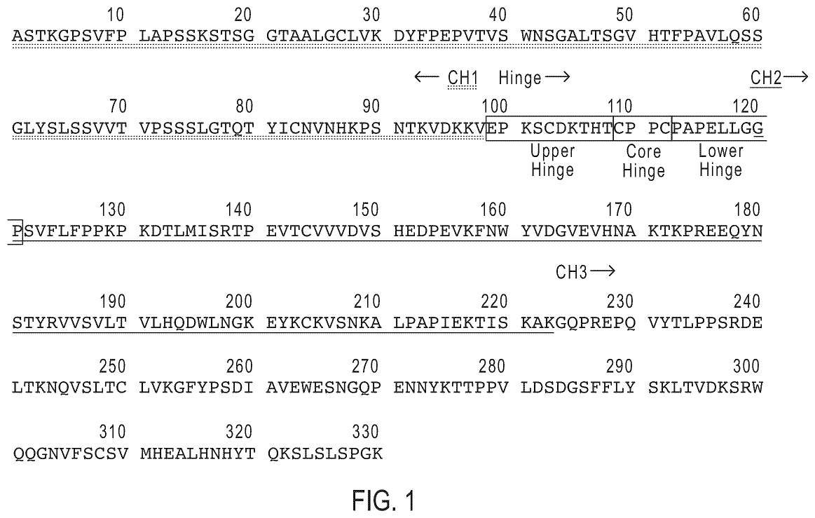

Alanine residues have usually been the preferred substituent for replacing a natural amino acid with an unnatural one so as to reduce function because alanine has a side chain without any functional groups. For example, the well-known technique of alanine-scanning mutagenesis systematically replaces every natural residue in a protein or protein domain with alanine to identify which natural residues contribute primarily to function. Replacing an amino acid with a functional group with alanine eliminates the functional group and its contribution to binding to any receptor, but the presence of the alanine side chain substantially preserves conformation, reducing the potential for immunogenicity or other complexities due to conformational changes. An alternative strategy replaces amino acids in the lower hinge region of a chimeric IgG comprising IgG1 upper hinge, IgG1 lower hinge, IgG4 CH2, and IgG1 CH3 with corresponding amino acids from the human IgG2 isotype so as to reduce FcγR binding without unacceptable conformational changes and consequent immunogenicity (see WO14/121087). In yet another alternative strategy, amino acid positions 233-236 (EU numbering) of a modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2).

IgG1 heavy chain constant regions with a hinge region modified to reduce Fc receptor and/or effector function are provided. The modification occurs at amino acid positions 233-236 (EU numbering) by substitution/deletion of ELLG (SEQ ID NO: 23) with PVA-, where amino acid 236 is deleted. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

As presented in the instant Examples, it was found that this substitution/deletion mutation (ELLG (SEQ ID NO: 23)→PVA—at amino acid positions 233-236 (EU numbering)) in an IgG1 background maximized hinge flexibility while reducing FcR engagement and minimizing immunogenicity, thereby providing an alternative chimeric heavy chain constant region for inclusion in, for example therapeutic antibodies and Fc fusion proteins. Having alternatives to those chimeric heavy chain constant regions known in the art is valuable. Having alternative chimeric heavy chain constant regions based on different IgG subclasses allows for, e.g., antibody activity optimization. This modified immunoglobulin constant region can be incorporated into virtually any format of antibody or Fc fusion protein. Such antibodies or fusion proteins can be used in methods of treatment, particularly methods of treatment in which the mechanisms of action of the antibody or Fc fusion protein is not primarily or at all dependent on effector functions, as is the case when an antibody inhibits a receptor-ligand interaction or agonizes a receptor.

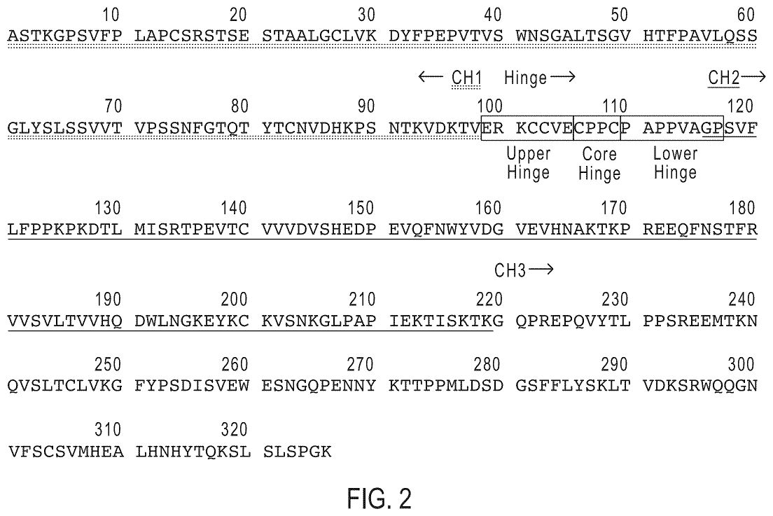

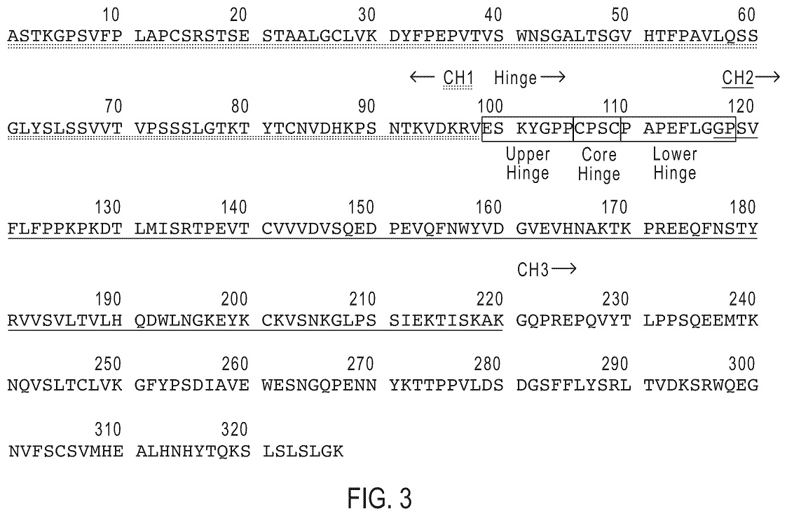

Excluding the ELLG (SEQ ID NO: 23)→PVA-substitution/deletion at amino acid positions 233-236 (EU numbering), a chimeric constant domain is considered to be of an IgG1 isotype if it differs from IgG1 by no more than 1, 2, 3, 4, 5, 6, 7, 8, 9, 10, 11, 12, 13, 14, 15, 16, 17, 18, 19, or 20 substitutions, deletions or insertions, except however, that the CH1 domain can optionally be omitted entirely, as can the upper hinge region. CH1, CH2 and CH3 domains are each considered to be of IgG1 isotype if differing from the CH1, CH2 and CH3 region of the IgG1 wild type sequence by no more than 1, 2, 3, 4, 5, 6, 7, 8, 9, or 10 substitutions, deletions, or insertions. Substitutions, deletion, and/or insertions (excluding the ELLG (SEQ ID NO: 23)→PVA- substitution/deletion at amino acid positions 233-236 (EU numbering)) can be any substitutions, deletions, and/or insertions. In some embodiments, the substitutions, deletion, and/or insertions do not result in a sequence identical to, e.g., CH1, CH2, or CH3 of another IgG isotype (e.g., IgG2, IgG3, or IgG4). For example, in some embodiments, if a chimeric constant domain of the disclosure comprises one or more of H268Q, K274Q, Y296F, A327G, A330S, and P331S (EU numbering), they do not all occur simultaneously. Wild type sequences for heavy chain constant regions of IgG1, IgG2, and IgG4 are depicted in FIGS. 1, 2, and 3, respectively, with delineation of CH1, hinge, CH2, and CH3 regions. Exemplary mutations that can be included in a chimeric constant domain of the disclosure are provided below.

The sequence of wild type IgG1 heavy chain constant region (amino acids 216-447; EU numbering)) comprises:

(SEQ ID NO: 1)
EPKSCDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SRDELTKNQVSLTCLVKGFYPSDIAVEWESNGQPENNYKTTPPVLD
SDGSFFLYSKLTVDKSRWQQGNVFSCSVMHEALHNHYTQKSLSLSP
GK,

where the upper hinge region comprises EPKSCDKTHT (SEQ ID NO: 24) (amino acids 216-225 (EU numbering); amino acids 1-10 of the wild type IgG1 constant domain sequence); the core hinge comprises CPPC (SEQ ID NO: 25) (amino acids 226-229 (EU numbering); amino acids 11-14 of the wild type IgG1 constant domain sequence); the lower hinge region comprises PAPELLG (SEQ ID NO: 26) (amino acids 230-236 (EU numbering); amino acids 15-21 of the wild type IgG1 constant domain sequence), CH2 comprises GPSVFLFPPKPKDTLMISRTPEVTCVVVDVSHEDPEVKFNWYVDGVEVHNAKTKPREEQYNST YRVVSVLTVLHQDWLNGKEYKCKVSNKALPAPIEKTISKAK (SEQ ID NO: 27) (amino acids 237-340 (EU numbering); amino acids 22-125 of the wild type IgG1 constant domain sequence), and CH3 comprises GQPREPQVYTLPPSRDELTKNQVSLTCLVKGFYPSDIAVEWESNGQPENNYKTTPPVLDSDGS FFLYSKLTVDKSRWQQGNVFSCSVMHEALHNHYTQKSLSLSPGK (SEQ ID NO: 28) (amino acids 341-447 (EU numbering): amino acids 126-232 of the wild type IgG1 constant sequence). It will be recognized by those skilled in the art that amino acids 237-238 (EU numbering) represents both the C-terminus of the lower hinge and the N-terminus of the CH2 region. For the purposes of the instant disclosure, however, amino acids 237-238 (EU numbering) are shown as part of the lower hinge.

In some embodiments, the sequence of the chimeric constant region of the disclosure, also termed IqG1 PVA herein (amino acids 216-447; EU numbering)) comprises:

(SEQ ID NO: 2)
EPKSCDKTHTCPPCPAPPVAGPSVFLFPPKPKDTLMISRTPEVTCV
VVDVSHEDPEVKFNWYVDGVEVHNAKTKPREEQYNSTYRVVSVLTV
LHQDWLNGKEYKCKVSNKALPAPIEKTISKAKGQPREPQVYTLPPS
RDELTKNQVSLTCLVKGFYPSDIAVEWESNGQPENNYKTTPPVLDS
DGSFFLYSKLTVDKSRWQQGNVFSCSVMHEALHNHYTQKSLSLSPG
K,

where the upper hinge region comprises EPKSCDKTHT (SEQ ID NO: 56) (amino acids 216-225 (EU numbering); amino acids 1-10 of the IgG1 PVA constant domain sequence); the core hinge comprises CPPC (SEQ ID NO: 57) (amino acids 226-229 (EU numbering): amino acids 11-14 of the IgG1 PVA constant domain sequence); the lower hinge region comprises PAPPVA (SEQ ID NO: 29) (amino acids 230-236 (EU numbering); amino acids 15-20 of the IgG1 PVA constant domain sequence), CH2 comprises GPSVFLFPPKPKDTLMISRTPEVTCVVVDVSHEDPEVKFNWYVDGVEVHNAKTKPREEQYNST YRVVSVLTVLHQDWLNGKEYKCKVSNKALPAPIEKTISKAK (SEQ ID NO: 58) (amino acids 237-340 (EU numbering): amino acids 21-124 of the IgG1 PVA sequence), and CH3 comprises GQPREPQVYTLPPSRDELTKNQVSLTCLVKGFYPSDIAVEWESNGQPENNYKTTPPVLDSDGS FFLYSKLTVDKSRWQQGNVFSCSVMHEALHNHYTQKSLSLSPGK (SEQ ID NO: 59) (amino acids 341-447 (EU numbering); amino acids 125-231 of the IgG1 PVA sequence).

In some aspects, the chimeric constant domain comprises an amino acid sequence having at least 90%, at least 91%, at least 92%, at least 93%, at least 94%, at least 95%, at least 96%, at least 97%, at least 98%, at least 98%, or 100% sequence identity with:

(SEQ ID NO: 2)
EPKSCDKTHTCPPCPAPPVAGPSVFLFPPKPKDTLMISRTPEVTCV
VVDVSHEDPEVKFNWYVDGVEVHNAKTKPREEQYNSTYRVVSVLTV
LHQDWLNGKEYKCKVSNKALPAPIEKTISKAKGQPREPQVYTLPPS
RDELTKNQVSLTCLVKGFYPSDIAVEWESNGQPENNYKTTPPVLDS
DGSFFLYSKLTVDKSRWQQGNVFSCSVMHEALHNHYTQKSLSLSPG
K.

The chimeric constant domain can also include knob mutations, hole mutations, star mutations, disulfide bridge-forming mutations, etc., to facilitate heterodimerization and/or purification.

In some embodiments, the chimeric constant domain comprises an amino acid sequence having at least 97% sequence identity, at least 98% sequence identity, at least 99% sequence identity, or 100% sequence identity to the chimeric constant domain of SEQ ID NO:3 (IgG1 PVA).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:15 (IgG1 PVA with knob mutation T366W, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:16 (IgG1 PVA with hole mutations T366S, L368A, Y407V, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:17 (IgG1 PVA with knob mutation T366W and star mutations H435R, Y436F, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:18 (IgG1 PVA with hole mutations T366S, L368A, Y407V and star mutations H435R, Y436F, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:19 (IgG1 PVA with knob mutation T366W and Cys mutation S354C, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:20 (IgG1 PVA with hole mutations T366S, L368A, Y407V and Cys mutation S354C, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:21 (IgG1 PVA with knob mutation T366W, star mutations H435R, Y436F, and Cys mutation S354C, EU numbering).

In other embodiments, the chimeric constant domain comprises an amino acid sequence having at least 97% sequence identity, at least 98% sequence identity, at least 99% sequence identity, or 100% sequence identity to the chimeric constant domain of SEQ ID NO:22 (IgG1 PVA with hole mutations T336S, L368A, Y407V, star mutations H435R, Y436F, and Cys mutation S354C, EU numbering).

In some embodiments, the chimeric constant domain comprises a CH1 domain or a fragment thereof at the chimeric constant domain's N-terminus. In some embodiments, the CH1 domain comprises ASTKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKV (SEQ ID NO: 30) or a fragment or variant thereof, e.g., a variant having up to 1, 2, 3, 4, 5, 6, 7, 8, 9, or 10 substitutions, deletions, or insertions. In some embodiments, the chimeric constant domain comprises a CH1 fragment. In certain embodiments the CH1 fragment comprises or consists of DKKV (SEQ ID NO: 31). In other embodiments, the CH1 fragment comprises or consists of DKRV (SEQ ID NO: 32).

### 6.2.1.1. Modified Chimeric Constant Domains

In some embodiments the chimeric constant domain is modified to further alter effector function and/or provide for heterodimerization. Exemplary modifications are described in Sections 6.2.1.1.1 and 6.2.1.1.2.

6.2.1.1.1. Chimeric Constant Domains with Further Alteration of Effector Function

In some embodiments, the chimeric constant domain comprises one or more amino acid substitutions in addition to the ELLG (SEQ ID NO: 23)→PVA-substitution/deletion at amino acid positions 233-236 (EU numbering), with the additional amino acid substitutions further reducing binding to an Fc receptor and/or effector function.

In some embodiments, the Fc receptor is an Fey receptor. In certain embodiments, the Fc receptor is a human Fc receptor. In particular embodiments, the Fc receptor is an activating Fc receptor. In a specific embodiment, the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the chimeric constant domain comprises one or more amino acid substitution at one or more of positions L234, L235, G237, D265, N297, P329, A330, P331, and P329 (EU numbering).

In some embodiments, the chimeric constant domain comprises an amino acid substitution at position P329 (EU numbering). In a more specific embodiment, the amino acid substitution is P329A or P3290G, particularly P3290G (EU numbering). In one embodiment, the chimeric constant domain comprises an amino acid substitution at position P329 and a further amino acid substitution at position N297 and/or P331 (EU numbering). In a more specific embodiment, the further amino acid substitution is N297A or N297D and/or P331S.

In some embodiments, the chimeric constant domain comprises amino acid substitutions at positions G237, A330, and P331 (EU numbering). In a more specific embodiment, the amino acid substitutions are G237A, A330S, and P331S (numberings according to Kabat EU index).

In some embodiments, the chimeric constant domain comprises D265A and N297A mutations (EU numbering) to reduce effector function.

When incorporated into an antibody or fusion protein, the same one or more additional amino acid substitutions can be present in each of the two chimeric constant domains included in the antibody or fusion protein. For example, in a particular embodiment, each chimeric constant domain comprises the amino acid substitutions G237A, A330S, and P331S (EU numbering), i.e., in each of the first and the second chimeric constant domains, the glycine residue at position 237 is replace with an alanine residue (G237A), the alanine residue at position 330 is replaced with a serine residue (A330S), and the proline residue at position 331 is replaced with a serine residue (P331S) (EU numbering).

### 6.2.1.1.2. Chimeric Constant Domain Heterodimerization Variants

In certain embodiments, recombinant polypeptides of the disclosure dimerize via the chimeric constant domain. In some embodiments, the resulting dimer forms or forms a part of an antibody or an Fc fusion protein. In certain embodiments, a first recombinant polypeptide dimerizes with an identical second recombinant polypeptide, forming a homodimer. In other embodiments, a first recombinant polypeptide dimerizes with a second recombinant polypeptide that is not identical to the first recombinant polypeptide, forming a heterodimer. For example, a first recombinant polypeptide can include a first target binding domain specific for a first target molecule, and the second recombinant polypeptide can include a second target binding domain specific for a second target molecule. When the first and second recombinant polypeptides dimerize via their respective chimeric constant domains, the result is a heterodimer, Inadequate heterodimerization of recombinant polypeptides (e.g., heterodimerization of the chimeric constant domain of each recombinant polypeptide to form an Fc region) can be an obstacle for increasing the yield of desired heterodimeric molecules and represents challenges for purification. A variety of approaches available in the art can be used in for enhancing dimerization of recombinant polypeptides of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides heterodimers comprising recombinant polypeptides of the disclosure. In some embodiments, the heterodimer is an antibody. In other embodiments, the heterodimer is an Fc fusion protein. Heterodimerization of the recombinant polypeptides via the CH3 domains of the chimeric constant domains can give rise to a desired heterodimer (e.g., an antibody or fusion protein), while homodimerization of identical chimeric constant domains will reduce yield. Thus, in some embodiments, the recombinant polypeptides that associate to form an antibody or fusion protein of the disclosure will comprise chimeric constant domains which contain CH3 domains with modifications that favor heterodimeric association relative to unmodified constant domains.

In a specific embodiment said modification promoting the formation of heterodimers is a so-called “knob-into-hole” or “knob-in-hole” modification, comprising a “knob” modification in one of the chimeric constant domains and a “hole” modification in the other chimeric constant domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g, tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of a first chimeric constant domain is replaced with an amino acid residue having a larger side chain volume, thereby generating a protuberance within the CH3 domain of the first chimeric constant domain, which is positionable in a cavity within the CH3 domain of a second chimeric constant domain, and an amino acid residue in the CH3 domain of the second chimeric constant domain is replaced with an amino acid residue having a smaller side chain volume, thereby generating a cavity within the CH3 domain of the second chimeric constant domain within which the protuberance within the CH3 domain of the first chimeric constant domain is positionable. Preferably said amino acid residue having a larger side chain volume is selected from the group consisting of arginine (R), phenylalanine (F), tyrosine (Y), and tryptophan (W), Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis.

In a specific embodiment, in the first chimeric constant domain the threonine residue at position 366 is replaced with a tryptophan residue (T366W), and in the second chimeric constant domain the tyrosine residue at position 407 is replaced with a valine residue (Y407V) and optionally, the threonine residue at position 366 is replaced with a serine residue (T366S) and the leucine residue at position 368 is replaced with an alanine residue (L368A) (EU numbering), In a further embodiment, in the first chimeric constant domain, additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second chimeric constant domain, additionally the tyrosine residue at position 349 is replaced by a cysteine residue (Y3490C) (EU numbering). In a particular embodiment, the first chimeric constant domain comprises the amino acid substitutions S354C and T366W, and the second Fc domain comprises the amino acid substitutions Y3490C, T366S, L368A and Y407V (EU numbering).

In some embodiments, electrostatic steering (e.g., as described in Gunasekaran et aL, 2010, J Biol Chem 285(25): 19637-46) can be used to promote the association of the first and the second chimeric constant domains.

As an alternative, or in addition, to the use of chimeric constant domains that are modified to promote heterodimerization, a chimeric constant domain can be modified to allow a purification strategy that enables selections of chimeric constant domain heterodimers. In one such embodiment, one recombinant polypeptide comprises a modified chimeric constant domain that abrogates its binding to Protein A, thus enabling a purification method that yields a heterodimeric protein. See, for example, U.S. Pat. No. 8,586,713. As such, an antibody or fusion protein can comprise a first CH3 domain and a second CH3 domain, wherein the first and second CH3 domains differ from one another by at least one amino acid, and wherein the at least one amino acid difference reduces binding of the antibody or fusion protein to Protein A as compared to a corresponding antibody or fusion protein lacking the amino acid difference. In one embodiment, the first CH3 domain binds Protein A and the second CH3 domain contains a mutation/modification that reduces or abolishes Protein A binding such as an H435R modification (EU numbering). The second CH3 may further comprise a Y436F modification (EU numbering). This class of modifications is referred to herein as “star” mutations.

In some embodiments, the chimeric constant domain can contain one or more mutations (e.g., knob and hole mutations) to facilitate heterodimerization as well as star mutations to facilitate purification.

### 6.2.2. Antibodies

In some embodiments, recombinant polypeptides comprising chimeric constant domains dimerize to form an antibody. The recombinant polypeptides can be used in any type of engineered antibody, including chimeric, humanized, veneered, or human antibodies. The antibody can be a monoclonal antibody or a genetically engineered polyclonal antibody.

The antibodies of the disclosure typically comprise or consist of recombinant polypeptides of the disclosure, the recombinant polypeptides each comprising a chimeric constant domain and one or more antigen binding domains and optionally, one or more linkers separating one or more domains in the recombinant polypeptides.

An antibody can be composed of one or more recombinant polypeptides of the disclosure. In some embodiments, the antibody is composed of two recombinant polypeptides, optionally in association with one or more additional polypeptide chains (e.g., a polypeptide chain comprising the light chain of an antigen binding domain, such as in the case of a Fab). The two recombinant polypeptides can be identical, thereby forming a homodimer, or different, thereby forming a heterodimer. The two recombinant polypeptides are generally configured to dimerize together via the chimeric constant domain of each recombinant polypeptide. In some embodiments, one or more additional polypeptide chains associate with the dimerized recombinant polypeptides. Thus, an antibody may include two, three, four or more polypeptide chains.

Where the two recombinant polypeptides include antigen binding domains that target the same epitope on the same target molecule, the antibody is monospecific. Where the two recombinant polypeptides, either individually or together, include antigen binding domains that target two or more different epitopes on the same target molecule, or two or more different target molecules, the antibody or fusion protein is multispecific (e.g., bispecific ). Most antibodies are multimeric by virtue of dimerization via the chimeric constant domains of the recombinant polypeptides. A multispecific antibody or fusion protein can be monovalent for a first antigen binding domain and monovalent for a second and subsequent antigen binding domains, or can be monovalent for a first antigen binding domain, multivalent for a second antigen binding domain, and monovalent or multivalent for any additional (e.g., third) antigen binding domain.

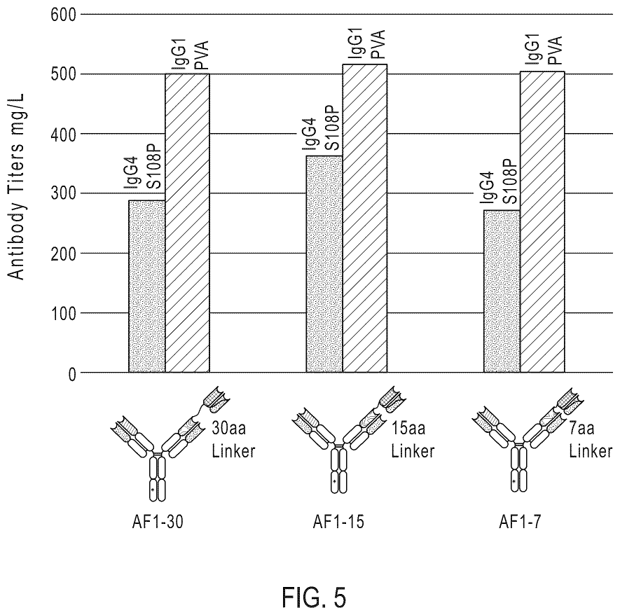

Particularly preferred formats of antibodies comprising the constant domains of the disclosure are N-terminal scFv formats (e.g., as described in WO 2021/091953 A1, the contents of which are incorporated by reference herein in their entireties; examples of N-terminal scFv formats are shown in FIG. 5 herein) or antibodies in which the N-terminal scFv is replaced with an N-terminal Fab. scFv and Fab domains are described in Sections 6.2.4.2 and 6.2.4.1, respectively. In some embodiments, the antibodies are trispecific and are in the “2+1 N-scFv” or “2+1 N-Fab” format, in which a traditional bispecific antibody has an scFv or Fab domain appended to the N-terminus of one of its VH domains, preferably via a linker. Suitable linkers are disclosed in Section 6.2.5.

As disclosed in WO 2021/091953 A1, the “2+1 N-scFv” format typically comprises: (a) a first polypeptide chain comprising, in an N- to C-terminal orientation, (i) an scFv comprising a first antigen binding site (“ABS1”) operably linked to (ii) a first heavy chain region of a first Fab (“Fab1”) operably linked to (iii) an Fc domain; (b) a second polypeptide chain comprising, in an N- to C-terminal orientation, (i) a second heavy chain region of a second Fab (“Fab2”) operably linked to (ii) an Fc domain; (c) a third polypeptide chain comprising a first light chain that pairs with the first heavy chain region to form Fab1, wherein Fab1 comprises a second antigen binding site (“ABS2”); and (d) a fourth polypeptide chain comprising a second light chain that pairs with the second heavy chain region to form Fab2, wherein Fab2 comprises a third antigen binding site (“ABS3”). In some embodiments, ABS1, ABS2, and ABS3 each bind to a different epitope. In some embodiments, two of ABS1, ABS2 and ABS3 specifically bind to different epitopes of the same target molecule. In some embodiments, the scFv, Fab1 and Fab2 are capable of specifically binding their respective targets at the same time. In some embodiments, at least one of ABS1, ABS2 and ABS3 specifically binds to a target molecule with a first tissue expression profile and at least one of ABS1, ABS2 and ABS3 specifically binds to a target molecule with a second tissue expression profile that is overlapping with, but not identical to, the first tissue expression profile. In some embodiments, the scFv is linked to the first heavy chain region via a linker. In some embodiments, the antigen binding molecule is trivalent. In some embodiments, the scFv is linked to the first heavy chain region via a linker. In some embodiments, the linker is at least 5, 6, 7, or more amino acids in length. In some embodiments, the linker is up to 30, up to 40, or up to 50 amino acids in length.

In some embodiments, effector functions dependent on Fcγ receptor binding, such as ADCC or ADCP, are reduced by, for example, at least 90%, at least 95%, at least 99%, or to background levels relative to an antibody comprising a wild type IgG1 constant heavy chain domain. Such functions include cell killing or phagocytosis, B-cell activation, and release of inflammatory mediators, such as cytokines. Some such effects can be quantified by measurement of EC50. In certain embodiments, an antibody comprising the chimeric constant domain of the disclosure exhibits cytotoxic activity of less than 20% cytolysis (e.g., % cytotoxicity), less than 10% cytolysis, less than 5% cytolysis, less than 4% cytolysis, less than 3% cytolysis, less than 2% cytolysis, or 0% cytolysis or undetectable cytolysis, as measured in an in vitro or ex vivo cell killing assay compared with suitable isotype-matched control antibodies with a wild type constant region, optionally, measured at an antibody concentration of at least 10 nM.

In some embodiments an antibody comprising a recombinant polypeptide of the disclosure exhibits at least 5-fold reduced binding, at least 10-fold reduced binding, at least 50-fold reduced binding, at least 100-fold reduced binding, at least 500-fold reduced binding, at least 1,000-fold reduced binding, at least 5,000-fold reduced binding, or at least 10,000-fold reduced binding to a human Fc receptor (FcR) than an antibody comprising a wild type IgG1 constant domain. The FcR can be, for example, FcRγ1, FcRγ2A, FcRγ2B, FcRγ3A, or FcRγ3B. FcR binding can be determined, for example, by ELISA as described in Section 8.1.4.

In some embodiments, binding affinity of an antibody incorporating a chimeric constant domain of the disclosure to a target is not substantially affected by the chimeric constant domain relative to an appropriate heavy chain constant domain (e.g., wild type IgG1). That is, the binding affinity is typically the same within experimental error or at least within a factor of 2 or 3 of a suitable control antibody with an isotype-matched wild type constant domain. The same is the case for functional properties not dependent on FcγR binding, such as ability to inhibit receptor-ligand binding (e.g., EC50), or ability to agonize a receptor.

Immunogenicity of antibodies incorporating recombinant polypeptides of the disclosure (and thus chimeric constant domains of the disclosure) compared with isotype matched controls can be assessed in vitro from dendrocyte maturation or T-cell proliferation on challenge (Gaitonde et al., 2011, Methods Mol Biol 716:267-80) or in vivo by comparing incidence of reactive antibodies against administered antibodies between populations. In some embodiments, the immunogenicity of antibodies incorporating recombinant polypeptides of the disclosure (and thus chimeric constant domains of the disclosure) is reduced. In other embodiments, the immunogenicity is not significantly different from the isotype matched controls or not worse than 2, 3, or 5-fold greater than the isotype matched control. Likewise, pharmacokinetic parameters such as Cₘₐₓ, C_{average}, area under the curve and half-life are preferably not significantly different or at least not lower by a factor of no more than 2, 3 or 5 than isotype matched controls. Substantial retention or improvement of such PK parameters can provide an indication that antibodies of the disclosure have not undergone substantial conformational changes triggering enhanced removal mechanisms.

In certain embodiments, the antibody of the disclosure exhibits increased expression in an expression system compared to an antibody comprising a wild type IgG1 constant domain (e.g., comprising an IgG1 hinge, IgG1 CH2, and IgG1 CH3). In some embodiments the increase compared to the antibody comprising the wild type IgG1 heavy chain constant domain is at least a 5% increase, at least a 10% increase, at least a 10% increase, at least a 20% increase, at least a 25% increase, at least a 30% increase, at least a 40% increase, at least a 50% increase, at least a 60% increase, at least a 70% increase, at least a 30% increase, at least a 90% increase, or at least a 100% increase. In certain aspects, increased expression of an antibody is evidenced by increased production of a secreted protein such as an antibody and/or increased total protein yield obtained from an in vitro expression system, e.g., an antibody expression system. In some embodiments, the expression system is a Chinese hamster ovary (CHO) stable expression system. Increased expression in a CHO stable expression system can be suitably evaluated as described in Section 8.1.6, with protein production stimulated via induction of cultures with Doxycycline for multiple days. In other embodiments, the expression system is an HEK293-based expression system, e.g., an Expi293F™ expression system.

### 6.2.3. Fusion Proteins

In some embodiments, recombinant polypeptides comprising chimeric constant domains dimerize to form a fusion protein. Fusion proteins of the disclosure typically comprise or consist of recombinant polypeptides of the disclosure, the recombinant polypeptides each comprising a chimeric constant domain and one or more heterologous polypeptides and optionally one or more linkers separating one or more domains in the recombinant polypeptide.

A fusion protein can be composed of one or more recombinant polypeptides of the disclosure. In some embodiments, the fusion protein is composed of two recombinant polypeptides. The two recombinant polypeptides can be identical, thereby forming a homodimer, or different, thereby forming a heterodimer. The two recombinant polypeptides are generally configured to dimerize together via the chimeric constant domain of each recombinant polypeptide. In some embodiments, one or more additional polypeptide chains associate with the dimerized recombinant polypeptides. Thus, a fusion protein may include two, three, four or more polypeptide chains.

In some embodiments, a recombinant polypeptide comprises a chimeric constant domain and a heterologous polypeptide. In some embodiments, a heterologous polypeptide in a fusion protein is a polypeptide not naturally linked to an immunoglobulin constant domain, including engineered forms of antigen binding domains. The heterologous polypeptide can be any other proteinaceous molecule of interest. Exemplary heterologous polypeptides include but are not limited to cytokines, ligands, peptidic antigens against a pathogen, extracellular receptor domains and functional fragments thereof (e.g., soluble receptors), and non-naturally occurring antigen binding domains (e.g., engineered antigen binding domains such as scFvs, domain swapped Fabs). Thus, in some embodiments, a fusion protein can be an antibody. Exemplary receptor proteins whose extracellular domains can be combined with a chimeric constant domain in a recombinant polypeptide are known in the art (see, e.g., Klinkert, et al., 1997, J Neuroimmunol 72(2):163-8; Milligan et aL, 2004, Curr Pharm Des 10(17):1989-2001; and Schwache & Muller-Newen, 2012, Eur J Cell Biol 91(6-7):428-34).

In some embodiments, a fusion protein is homodimeric. In other embodiments, a fusion protein is heterodimeric.

In certain embodiments a fusion protein is monospecific (e.g., comprises a target binding domain(s) specific for a single target molecule, or comprises a single ligand domain), In other embodiments, a fusion protein is multispecific (e.g., comprises two or more target binding domains each specific for different target molecule, or comprises two or more different ligand domains). Most fusion proteins are multimeric by virtue of dimerization via the chimeric constant domains of the recombinant polypeptides.

In some embodiments, effector functions dependent on Fcγ receptor binding, such as ADCC or ADCP, are reduced by, for example, at least 90%, at least 95%, at least 99%, or to background levels relative to a fusion protein comprising a wild type IgG1 constant heavy chain domain. Such functions include cell killing or phagocytosis, B-cell activation, and release of inflammatory mediators, such as cytokines. Some such effects can be quantified by measurement of EC50. In certain embodiments, a fusion protein comprising the chimeric constant domain of the disclosure exhibits cytotoxic activity of less than 20% cytolysis (e.g., % cytotoxicity), less than 10% cytolysis, less than 5% cytolysis, less than 4% cytolysis, less than 3% cytolysis, less than 2% cytolysis, or 0% cytolysis or undetectable cytolysis, as measured in an in vitro or ex vivo cell killing assay compared with suitable isotype-matched control fusion protein with a wild type constant region, optionally, measured at a fusion protein concentration of at least 10 nM.

In some embodiments a fusion protein comprising a recombinant polypeptide of the disclosure exhibits at least 5-fold reduced binding, at least 10-fold reduced binding, at least 50-fold reduced binding, at least 100-fold reduced binding, at least 500-fold reduced binding, at least 1,000-fold reduced binding, at least 5,000-fold reduced binding, or at least 10,000-fold reduced binding to a human Fc receptor (FcR) than a fusion protein comprising a wild type IgG1 constant domain. The FcR can be, for example, FcRγ1, FcRγ2A, FcRγ2B, FcRγ3A, or FcRγ3B. FcR binding can be determined, for example, by ELISA as described in Section 8.1.4.

In some embodiments, binding affinity of a fusion protein incorporating a chimeric constant domain of the disclosure to a target is not substantially affected by the chimeric constant domain relative to an appropriate heavy chain constant domain (e.g., wild type IgG1). That is, the binding affinity is typically the same within experimental error or at least within a factor of 2 or 3 of a suitable control fusion protein with an isotype-matched wild type constant domain. The same is the case for functional properties not dependent on FcγR binding, such as ability to inhibit receptor-ligand binding (e.g., EC50), or ability to antagonize a receptor.

In some embodiments, the immunogenicity of fusion proteins incorporating recombinant polypeptides of the disclosure (and thus chimeric constant domains of the disclosure) is reduced. In other embodiments, the immunogenicity is not significantly different from the isotype matched controls or not worse than 2, 3, or 5-fold greater than the isotype matched control. Likewise, pharmacokinetic parameters such as Cₘₐₓ, C_{average}, area under the curve and half-life are preferably not significantly different or at least not lower by a factor of no more than 2, 3 or 5 than isotype matched controls. Substantial retention or improvement of such PK parameters can provide an indication that fusion proteins of the disclosure have not undergone substantial conformational changes triggering enhanced removal mechanisms.

In certain embodiments, the fusion protein of the disclosure may exhibit increased expression in an expression system compared to a fusion protein comprising a wild type IgG1 constant domain (e.g., comprising an IgG1 hinge, IgG1 CH2, and IgG1 CH3). In some embodiments the increase compared to the fusion protein comprising the wild type IgG1 heavy chain constant domain is at least a 5% increase, at least a 10% increase, at least a 10% increase, at least a 20% increase, at least a 25% increase, at least a 30% increase, at least a 40% increase, at least a 50% increase, at least a 60% increase, at least a 70% increase, at least a 80% increase, at least a 90% increase, or at least a 100% increase. In certain aspects, increased expression of a fusion protein is evidenced by increased production of a secreted protein such as a fusion protein and/or increased total protein yield obtained from an in vitro expression system, e.g., a fusion protein expression system. In some embodiments, the expression system is a Chinese hamster ovary (CHO) stable expression system. Increased expression in a CHO stable expression system can be suitably evaluated as described in Section 8.1.6, with protein production stimulated via induction of cultures with Doxycycline for multiple days. In other embodiments, the expression system is an HEK293-based expression system, e.g., an Expi293F expression system.

### 6.2.4. Target Binding Domains

Recombinant polypeptides of the disclosure can comprise one or more target binding domains (e.g., one, two, three, or more target binding domains), with each target binding domain binding specifically to a selected target molecule. In some embodiments, the recombinant polypeptide comprises a single target binding domain. The target binding domain can be N-terminal to the chimeric constant domain or can be C-terminal to the chimeric constant domain. In some embodiments, the single target binding domain is attached to the chimeric constant domain via a linker.

In some embodiments, the recombinant polypeptide comprises two or more target binding domains. The two or more target binding domains can be identical, or can be different. In some embodiments, the two or more target binding domains are identical, and bind the same epitope on a target molecule. In other embodiments, the two or more target binding domains are different, and either bind different epitopes on a target molecule, or bind to different target molecules. In some embodiments, the recombinant polypeptide comprises three or more target binding domains. In such embodiments, two of the target binding domains can be identical and bind to the same epitope on a first target molecule, with the remaining target binding domain(s) being different and binding to a different epitope on the first target molecule or binding to a different target molecule. When a recombinant polypeptide comprises two or more target binding domains, each of the two or more target binding domains can be N-terminal to the chimeric constant domain, each of the two or more target binding domains can be C-terminal to the chimeric constant domain, or one (or more) target binding domain(s) can be N-terminal to the chimeric constant domain with the other target binding domain(s) being C-terminal to the chimeric constant domains. The two or more target binding domains and the chimeric constant domain can be separated via one or more linkers.

Target binding domains can be specific for any epitope and/or target molecule of interest. Target molecules can be human, mammalian or bacterial. Targets can be antigens, such as proteins, glycoproteins, and carbohydrates from microbial pathogens, both viral and bacterial, and tumors.

Target binding domains can be an antigen binding domain or an antigen binding fragment of an antibody. In some embodiments, an antigen binding domain is that of a commercial antibody.

In certain aspects, a target binding domain included in a recombinant polypeptide of the disclosure can be any type of antibody fragment that specifically binds to a selected target molecule or epitope thereof. Antibody fragments include, but are not limited to, VH (or V_{H}) fragments, VL (or V_{L}) fragments, Fab fragments, F(ab′)₂ fragments, scFv fragments, Fv fragments, minibodies, diabodies, triabodies, and tetrabodies. Where an antigen binding domain includes two separate polypeptide chains (e.g., a Fab), a first polypeptide chain (e.g., comprising VH) can be included in the recombinant polypeptide and a second polypeptide chain (e.g., comprising VL) can be included in a polypeptide capable of associating with the VH included in the recombinant polypeptide. Exemplary targets for antibody molecules comprising constant domains of the disclosure are cell-surface expressed antigens such as proteins, carbohydrates, and lipids. In some embodiments, the target molecules are protein molecules. Exemplary target molecules include human klotho beta (“KLB”), human fibroblast growth factor receptor 1c isoform (“FGFR1c”), human fibroblast growth factor receptor 3 (“FGFR3”), human CD63, and human amyloid precursor-like protein 2 (APLP2). Exemplary KLB targeting domains are set forth in Tables 2A and 2B of WO 2021/091953 A1. Exemplary FGFR1c targeting domains are set forth in Tables 3A and 3B of WO 2021/091953 A1. Exemplary FGFR3 binding domains are set forth in Table 4 of WO 2021/091953 A1. Exemplary APLP2 binding domains are set forth in Table 5 of WO 2021/091953 A1. Exemplary CD3 binding domains are set forth in Table 6 of WO 2021/091953 A1. The binding domains can be configured in the 2+1 N-terminal scFv formats described in WO 2021/091953 A1 or a 2+1 N-Fab format in which the N-terminal scFv of WO 2021/091953 is replaced with an N-terminal Fab. The contents of WO 2021/091953 A1 are incorporated by reference herein in their entireties.

In other aspects, a target binding domain included in a recombinant polypeptide of the disclosure can be any type of receptor or target binding portion thereof that specifically binds to a selected target molecule.

### 6.2.4.1. Fab

Fab domains were traditionally produced by proteolytic cleavage of immunoglobulin molecules using enzymes such as papain. In antibodies comprising recombinant polypeptides of the disclosure, the Fab domains are typically recombinantly expressed with at least a portion of the Fab being included in the recombinant polypeptide.

The Fab domains can comprise constant domain and variable region sequences from any suitable species, and thus can be murine, chimeric, human, or humanized. In some embodiments, variable regions sequences and/or constant domain region sequences are derived from a known antibody. Examples of known antibodies are provided above.

Fab domains typically comprise a CH1 domain attached to a VH domain which pairs with a CL domain attached to a VL domain. In a wild type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For antibodies comprising recombinant polypeptides of the disclosure, particularly when the light chain is not a common or universal light chain, it is advantageous to use Fab heterodimerization strategies to permit the correct association of Fab domains belonging to the same antigen binding domain and minimize aberrant pairing of Fab domains belonging to different antigen binding domains. For example, the Fab heterodimerization strategies shown in Table 1 below can be used:

TABLE 1
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20:472-86; |
| | | | | | PMID:22014573 |
| orthogonal Fab | 39K, 62E | H172A, | 1R, 38D, | L135Y, | Lewis et al., 2014, Nat |
| VHVRD1CH1CRD2- | | F174G | (36F) | S176W | Biotechnol 32:191-8 |
| VLVRD1CλCRD2 | | | | | |
| orthogonal Fab | 39Y | WT | 38R | WT | Lewis et al., 2014, Nat |
| VHVRD2CH1wt- | | | | | Biotechnol 32:191-8 |
| VLVRD2Cλwt | | | | | |
| TCR CαCβ | 39K | TCR Cα | 38D | TCR Cβ | Wu et al., 2015, MAbs |
| | | | | | 7:364-76 |
| CR3 | WT | T192E | WT | N137K, | Golay at al., 2016, J |
| | | | | S114A | Immunol 196:3199-211. |
| MUT4 | WT | L143Q, | WT | V133T, | Golay at al., 2016, J |
| | | S188V | | S176V | Immunol 196:3199-211. |
| DuetMab | WT | F126C | WT | S121C | Mazor et al., 2015, MAbs |
| | | | | | 7:377-89, Mazor et al., |
| | | | | | 2015, MAbs 7:461-669. |
| Domain | WT | CH3 + knob or | WT | CH3 + hole or | Wozniak-Knopp et al., |
| exchanged | | hole mutation | | knob mutation | 2018, |
| | | | | | PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions, or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay at al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, e.g., Mazor at al., 2015, MAbs 7:377-89).

Fab domains can also be modified by replacing the CH1 domain and CL domain with alternative domains that promote correct assembly. For example, Wu et alt, 2015, MAbs 7:364-76, describes substituting the CH1 domain with the constant domain of the a T cell receptor and substituting the CL domain with thebb domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of a recombinant polypeptide or antibody of the disclosure. In various embodiments, employing a common light chain as described herein reduces the number of inappropriate species recombinant polypeptide or antibody as compared to employing original cognate VLs. In various embodiments, the VL domains of the recombinant polypeptides or antibodies are identified from monospecific antibodies comprising a common light chain. In various embodiments, the VH regions of the recombinant polypeptides or antibodies comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39JK5 sequence or a rearranged human Vκ3-20Jκ1 sequence, and include somatically mutated (e.g., affinity matured) versions. See, for example, U.S. Pat. No. 10,412,940.

6.2.4.2. scFv

Single chain Fv or “scFv” antibody fragments comprise the VH and VL domains of an antibody in a single polypeptide chain, are capable of being expressed as a single chain polypeptide (e.g., a recombinant polypeptide of the disclosure), and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.2.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human, or humanized VH and VL sequences. In some embodiments, the scFv can comprise VH and VL sequences from a known antibody. Examples of known antibodies are provided above.

To create an scFv-encoding nucleic acid, the VH and VL-encoding DNA fragments are operably linked to another fragment encoding a linker, e.g., encoding any of the linkers described in Section 6.2.5 (typically a repeat of a sequence containing the amino acids glycine and serine, such as the amino acid sequence (Gly4-Ser)₃ (SEQ ID NO: 13), such that the VH and VL sequences can be expressed as a contiguous single-chain protein, with the VL and VH regions joined by the flexible linker (see, e.g, Bird et al., 1988, Science 242:423-426; Huston et al., 1988, Proc. Natl. Acad. Sci. USA 85:5879-5883; McCafferty et at, 1990, Nature 348:552-554).

### 6.2.5. Linkers

In certain aspects, the present disclosure provides recombinant polypeptides in which two or more components of the recombinant polypeptide are connected to one another by a peptide linker (sometimes referred to herein as a “linker” for convenience). By way of example and not limitation, linkers can be used to connect (a) a target binding domain and a chimeric constant domain; (b) a first target binding domain and a second target binding domain; or (c) different domains within a target binding domain (e.g., the VH and VL domains in an scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the recombinant polypeptides of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO: 33) or SGₙ (SEQ ID NO: 34), where n is an integer from 1 to 10, e.g., 1 2, 3, 4, 5, 6, 7, 8, 9 or 10. In one embodiment, the linker is or comprises a monomer or multimer of repeat of G4S (SEQ ID NO: 11) e.g., (GGGGS), (SEQ ID NO: 35).

Polyglycine linkers can suitably be used in the recombinant polypeptides of the disclosure. In some embodiments, a peptide linker comprises two consecutive glycines (2Gly), three consecutive glycines (3Gly), four consecutive glycines (4Gly) (SEQ ID NO: 36), five consecutive glycines (5Gly) (SEQ ID NO: 37), six consecutive glycines (6Gly) (SEQ ID NO: 38), seven consecutive glycines (7Gly) (SEQ ID NO: 39), eight consecutive glycines (8Gly) (SEQ ID NO: 40) or nine consecutive glycines (9Gly) (SEQ ID NO: 41).

### 6.3. Nucleic Acids and Host Cells

In another aspect, the disclosure provides nucleic acids encoding the chimeric constant domains, recombinant polypeptides, antibodies, and fusion proteins of the disclosure. In some embodiments, the chimeric constant domains, recombinant polypeptides, antibodies, and fusion proteins are encoded by a single nucleic acid. In other embodiments in which an antibody or fusion protein is composed of more than one polypeptide chain, the antibody or fusion protein can be encoded by a plurality (e.g., two, three, four or more) of nucleic acids.

A single nucleic acid can encode an antibody or fusion protein that comprises a single recombinant polypeptide chain, an antibody or fusion protein that comprises two or more polypeptide chains, or a portion of an antibody or fusion protein that comprises more than two polypeptide chains (for example, a single nucleic acid can encode two polypeptide chains of an antibody or fusion protein comprising three, four or more polypeptide chains, or three polypeptide chains of an antibody or fusion protein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a chimeric constant domain, recombinant polypeptide, antigen, or fusion protein comprising two or more polypeptide chains is encoded by two or more nucleic acids. The number of nucleic acids encoding a chimeric constant domain, recombinant polypeptide, antigen, or fusion protein can be equal to or less than the number of polypeptide chains in the chimeric constant domain, recombinant polypeptide, antigen, or fusion protein (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.3.1. Vectors

The disclosure provides vectors comprising nucleotide sequences encoding a chimeric constant domain, recombinant polypeptide, antigen, or fusion protein described herein, for example one or two of the polypeptide chains of an antibody. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering and/or purifying the expressed polypeptides are known to those skilled in the art, and may be varied or optimized depending upon the specific expression vector and mammalian host cell employed, based upon the present description.

### 6.3.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.4. Pharmaceutical Compositions

The antibodies and fusion proteins of the disclosure may be in the form of compositions comprising the antibody or fusion protein and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended use of the antibody or fusion protein and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an antibody or fusion protein of the disclosure per dose. The quantity of antibody or fusion protein included in a unit dose will depend on the disease being treated, as well as other factors as are well known in the art. Such unit dosages may be in the form of a lyophilized dry powder containing an amount of antibody or fusion protein suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity antibody or fusion protein suitable for a single administration.

The pharmaceutical compositions may also be supplied in bulk from containing quantities of antibody or fusion protein suitable for multiple administrations.

Pharmaceutical compositions may be prepared for storage as lyophilized formulations or aqueous solutions by mixing an antibody or fusion protein having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington The Science and Practice of Pharmacy, 23rd edition (Adejare, ed. 2020). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of antibody or fusion protein.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.4.1. Pharmaceutical Compositions for Delivery

An antibody or fusion protein of the disclosure can be delivered by any method useful for gene therapy, for example as mRNA or through viral vectors encoding the antibody or fusion protein under the control of a suitable promoter.

Exemplary gene therapy vectors include adenovirus- or AAV-based therapeutics. Non-limiting examples of adenovirus-based or AAV-based therapeutics for use in the methods, uses or compositions herein include, but are not limited to: rAd-p53, which is a recombinant adenoviral vector encoding the wild type human tumor suppressor protein p53, for example, for the use in treating a cancer (also known as Gendicine®, Genkaxin®, Qi et aL, 2006, Modern Oncology, 14:1295-1297); Ad5_d11520, which is an adenovirus lacking the E1B gene for inactivating host p53 (also called H101 or ONYX-015: see, e.g., Russell et al., 2012, Nature Biotechnology 30:658-670); AD5-D24-GM-CSF, an adenovirus containing the cytokine GM-CSF, for example, for the use in treating a cancer (Cerullo et at, 2010, Cancer Res. 70:4297); rAd-HSVtk, a replication deficient adenovirus with HSV thymidine kinase gene, for example, for the treatment of cancer (developed as Cerepro®, Ark Therapeutics, see e.g. U.S. Pat. No. 6,579,855: developed as ProstAtak™ by Advantagene; International PCT Appl. No. WO2005/049094); rAd-TNFα, a replication-deficient adenoviral vector expressing human tumor necrosis factor alpha (TNFα) under the control of the chemoradiation-inducible EGR-1 promoter, for example, for the treatment of cancer (TNFerade™, GenVec; Rasmussen et al., 2002, Cancer Gene Ther. 9:951-7; Ad-IFNβ, an adenovirus serotype 5 vector from which the E1 and E3 genes have been deleted expressing the human interferon-beta gene under the direction of the cytomegalovirus (CMV) immediate-early promoter, for example for treating cancers (BG00001 and H5.110CMVhIFN-β, Biogen; Sterman et al., 2010, Mol. Ther. 18:852-860).

The nucleic acid molecule (e.g., mRNA) or virus can be formulated as the sole pharmaceutically active ingredient in a pharmaceutical composition or can be combined with other active agents for the particular disease to be treated. Optionally, other medicinal agents, pharmaceutical agents, carriers, adjuvants, diluents can be included in the compositions provided herein. For example, any one or more of a wetting agents, emulsifiers, and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 7. SPECIFIC EMBODIMENTS

While various specific embodiments have been illustrated and described, it will be appreciated that various changes can be made without departing from the spirit and scope of the disclosure(s). The present disclosure is exemplified by the numbered Group A, Group B, Group C and Group D numbered embodiments set forth below. Unless otherwise specified, features of any of the numbered embodiments of a particular group are applicable mutatis mutandis to the numbered embodiments of the other groups.

### Group A Numbered Embodiments

1. A recombinant polypeptide comprising a chimeric constant domain comprising, from N-terminus to C-terminus:
(a) a chimeric immunoglobulin hinge comprising:
   (i) a human IgG1 upper hinge amino acid sequence EPKSCDKTHT (SEQ ID NO: 24) from positions 216 to 225 (EU numbering)
   (ii) a human IgG1 core hinge amino acid sequence CPPC (SEQ ID NO: 25) from positions 226 to 229 (EU numbering);
   (iii) a human IgG2 lower hinge amino acid sequence PCPAPPVAGP (SEQ ID NO: 42) from positions 230 to 238 (EU numbering);
(b) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A3270, A330S, and P331S (EU numbering), they do not occur simultaneously; and
(c) a CH3 domain comprising a human IgG1 CH3 domain amino acid sequence from position 341 to 447 (EU numbering) of human IgG1, optionally comprising one or more CH3 domain amino acid substitutions, optionally wherein the substitutions are selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing.

2. The recombinant polypeptide of embodiment 1, wherein the chimeric constant domain comprises or consists of an amino acid sequence at least 95% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

3. The recombinant polypeptide of embodiment 1 or embodiment 2, wherein the chimeric constant domain comprises or consists of an amino acid sequence at least 96% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

4. The recombinant polypeptide of any one of embodiments 1 to 3, wherein the chimeric constant domain comprises or consists of an amino acid sequence at least 97% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

5. The recombinant polypeptide of any one of embodiments 1 to 4, wherein the chimeric constant domain comprises or consists of an amino acid sequence at least 98% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

6. The recombinant polypeptide of any one of embodiments 1 to 5, wherein the chimeric constant domain comprises or consists of an amino acid sequence at least 99% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

7. The recombinant polypeptide of any one of embodiments 1 to 6, wherein the chimeric constant domain comprises or consists of the amino acid sequence of any one of SEQ ID NQ:3, SEQ ID NO:15, SEQ ID NO:16, SEQ ID NO:17, SEQ ID NO:18, SEQ ID NO:1, SEQ ID NO:20, SEQ ID NO:21, or SEQ ID NO:22.

8. The recombinant polypeptide of any one of embodiments 1 to 7, wherein the optional one or more CH2 domain amino acid substitutions reduce binding of the chimeric constant domain to an Fc receptor and/or reduces effector function.

9. The recombinant polypeptide any one of embodiments 1 to 8, wherein the optional one or more CH2 domain amino acid substitutions comprise an amino acid substitution at position G237 (EU numbering).

10. The recombinant polypeptide of any one of embodiments 1 to 9, wherein the optional one or more CH2 domain amino acid substitutions comprise an amino acid substitution at position D265 (EU numbering).

11. The recombinant polypeptide of embodiment 10, wherein the optional one or more CH2 domain amino acid substitutions comprise amino acid substitution D265A (EU numbering).

12. The recombinant polypeptide of any one of embodiments 1 to 11, wherein the optional one or more CH2 domain amino acid substitutions comprise an amino acid substitution at position N297 (EU numbering).

13. The recombinant polypeptide of embodiment 12, wherein the optional one or more CH2 domain amino acid substitutions comprise amino acid substitution N297A (EU numbering).

14. The recombinant polypeptide of embodiment 12 wherein the optional one or more CH2 domain amino acid substitutions comprise amino acid substitution N297D (EU numbering).

15. The recombinant polypeptide of any one of embodiments 1 to 14, wherein the optional one or more CH2 domain amino acid substitutions comprise an amino acid substitution at position P329 (EU numbering).

16. The recombinant polypeptide of embodiment 15, wherein the optional one or more CH2 domain amino acid substitutions comprise amino acid substitution P329A (EU numbering).

17. The recombinant polypeptide of embodiment 15, wherein the optional one or more CH2 domain amino acid substitutions comprise amino acid substitution P329G (EU numbering).

18. The recombinant polypeptide of any one of embodiments 1 to 17, wherein the optional one or more CH2 domain amino acid substitutions comprise an amino acid substitution at position A330 (EU numbering).

19. The recombinant polypeptide of any one of embodiments 1 to 18, wherein the optional one or more CH2 domain amino acid substitutions comprise an amino acid substitution at position P331 (EU numbering).

20. The recombinant polypeptide of embodiment 19, wherein the optional one or more CH2 domain amino acid substitutions comprise amino acid substitution P331S (EU numbering).

21. The recombinant polypeptide of any one of embodiments 1 to 20, wherein the optional one or more CH2 domain amino acid substitutions comprise or consist of amino acid substitutions at positions D265 and N297 (EU numbering).

22. The recombinant polypeptide of embodiment 21, wherein the optional one or more CH2 domain amino acid substitutions comprise or consist of D265A and N297A (EU numbering).

23. The recombinant polypeptide of any one of embodiments 1 to 22, wherein the optional one or more CH3 domain amino acid substitutions provide for heterodimeric association of the chimeric constant domain with another constant domain. 24. The recombinant polypeptide of any one of embodiments 1 to 23, wherein the optional one or more CH3 domain amino acid substitutions comprise an amino acid substitution at position S354 or at E357 (EU numbering).

25. The recombinant polypeptide of embodiment 24, wherein the optional one or more CH3 domain amino acid substitutions comprise amino acid substitution S354C or E357C (EU numbering).

26. The recombinant polypeptide of any one of embodiments 1 to 25, wherein the optional one or more CH3 domain amino acid substitutions comprise an amino acid substitution at position T366 (EU numbering).

27. The recombinant polypeptide of embodiment 26, wherein the optional one or more CH3 domain amino acid substitutions comprise amino acid substitution T366W (EU numbering).

28. The recombinant polypeptide of any one of embodiments 1 to 27, wherein the optional one or more CH3 domain amino acid substitutions comprise or consist of amino acid substitutions at positions S354 and T366 (EU numbering).

29. The recombinant polypeptide of embodiment 28, wherein the optional one or more CH3 domain amino acid substitutions comprise or consist of S354C and T366W (EU numbering).

30. The recombinant polypeptide of any one of embodiments 1 to 23, wherein the optional one or more CH3 domain amino acid substitutions comprise an amino acid substitution at position Y349 (EU numbering).

31. The recombinant polypeptide of embodiment 30, wherein the optional one or more CH3 domain amino acid substitutions comprise amino acid substitution Y349C (EU numbering).

32. The recombinant polypeptide of any one of embodiments 1 to 23, 30, or 31, wherein the optional one or more CH3 domain amino acid substitutions comprise an amino acid substitution at position Y407 (EU numbering).

33. The recombinant polypeptide of embodiment 32, wherein the optional one or more CH3 domain amino acid substitutions comprise amino acid substitution Y407V (EU numbering).

34. The recombinant polypeptide of embodiment 32 or embodiment 33, wherein the optional one or more CH3 domain amino acid substitutions comprise an amino acid substitution at position T366 (EU numbering).

35. The recombinant polypeptide of embodiment 34, wherein the optional one or more CH3 domain amino acid substitutions comprise amino acid substitution T366S (EU numbering).

36. The recombinant polypeptide of any one of embodiments 32 to 35, wherein the optional one or more CH3 domain amino acid substitutions comprise an amino acid substitution at position L368 (EU numbering).

37. The recombinant polypeptide of embodiment 36, wherein the optional one or more CH3 domain amino acid substitutions comprise amino acid substitution L368A (EU numbering).

38. The recombinant polypeptide of any one of embodiments 1 to 23, or 30 to 37, wherein the optional one or more CH3 domain amino acid substitutions comprise or consist of amino acid substitutions at positions Y349, T366, L368, and Y407 (EU numbering).

39. The recombinant polypeptide of embodiment 33, wherein the optional one or more CH3 domain amino acid substitutions comprise or consist of Y349C, T366S, L368A, and Y407V (EU numbering).

40. The recombinant polypeptide of any one of embodiments 1 to 39, wherein the optional one or more CH3 domain amino acid substitutions provide for selective purification of the recombinant polypeptide. 41. The recombinant polypeptide of embodiment 40, wherein the optional one or more CH3 domain amino acid substitutions comprise an amino acid substitution at position H435 (EU numbering).

42. The recombinant polypeptide of embodiment 41, wherein the optional one or more CH3 domain amino acid substitutions comprise amino acid substitution H435R (EU numbering).

43. The recombinant polypeptide of embodiment 40, wherein the optional one or more CH3 domain amino acid substitutions comprise an amino acid substitution at position Y436 (EU numbering).

44. The recombinant polypeptide of embodiment 41, wherein the optional one or more CH3 domain amino acid substitutions comprise amino acid substitution Y436F (EU numbering).

45. The recombinant polypeptide of any one of embodiments 1 to 44, wherein the recombinant polypeptide is an antigen-binding protein.

46. The recombinant polypeptide of any one of embodiments 1 to 45, further comprising an IgG1 CH1 domain or a fragment or variant thereof.

47. The recombinant polypeptide of embodiment 46, wherein the fragment of IgG1 CH1 comprises or consists of DKKV (SEQ ID NO: 31).

48. The recombinant polypeptide of embodiment 46, wherein the fragment of IgG1 CH1 comprises or consists of DKRV (SEQ ID NO: 32).

49. The recombinant polypeptide of any one of embodiments 1 to 48, comprising at least one target binding domain.

50. The recombinant polypeptide of any one of embodiments 1 to 49, comprising at least two target binding domains.

51. The recombinant polypeptide of any one of embodiments 1 to 50, comprising at least three target binding domains.

52. The recombinant polypeptide of any one of embodiments 49 to 51, wherein the at least one target binding domain comprises a Fab.

53. The recombinant polypeptide of any one of embodiments 49 to 52, wherein the at least one target binding domain comprises an scFv.

54. The recombinant polypeptide of any one of embodiments 49 to 53, comprising a Fab as a first target binding domain and an scFv as a target binding domain.

55. The recombinant polypeptide of any one of embodiments 49 to 54, comprising at least one target binding domain N-terminal to the chimeric constant domain.

56. The recombinant polypeptide of any one of embodiments 49 to 55, comprising at least one target binding domain C-terminal to the chimeric constant domain.

57. The recombinant polypeptide of any one of embodiments 49 to 56, comprising at least a first target binding domain N-terminal to the chimeric constant domain and at least a second target binding domain C-terminal to the chimeric constant domain.

58. The recombinant polypeptide of any one of embodiments 49 to 57, comprising from N- to C-terminus an scFv, a Fab, and the chimeric constant domain.

59. The recombinant polypeptide of any one of embodiments 1 to 58, wherein the recombinant polypeptide is or forms a part of an antibody, optionally a multispecific antibody.

60. The recombinant polypeptide of any one of embodiments 1 to 59, wherein the recombinant polypeptide is or forms a part of a monovalent antibody.

61. The recombinant polypeptide of any one of embodiments 1 to 59, wherein the recombinant polypeptide is or forms a part of a bivalent antibody.

62. The recombinant polypeptide of embodiment 61, wherein the recombinant polypeptide is or forms a part of a bispecific antibody.

63. The recombinant polypeptide of any one of embodiments 1 to 59, wherein the recombinant polypeptide is or forms a part of a trivalent antibody.

64. The recombinant polypeptide of embodiment 63, wherein the recombinant polypeptide is or forms a part of a bispecific antibody.

65. The recombinant polypeptide of embodiment 63, wherein the recombinant polypeptide is or forms a part of a trispecific antibody.

66. The recombinant polypeptide of any one of embodiments 1 to 59, wherein the recombinant polypeptide is or forms a part of a tetravalent antibody.

67. The recombinant polypeptide of embodiment 66, wherein the recombinant polypeptide is or forms a part of a bispecific antibody.

68. The recombinant polypeptide of embodiment 66, wherein the recombinant polypeptide is or forms a part of a trispecific antibody.

69. The recombinant polypeptide of any one of embodiments 1 to 49, wherein the recombinant polypeptide is or forms a part of a fusion protein.

70. The recombinant polypeptide of any one of embodiments 59 to 69, wherein the antibody or fusion protein exhibits cytotoxic activity of less than 20% cytolysis at an antibody or fusion protein concentration of at least 10 nM.

71. The recombinant polypeptide of any one of embodiments 59 to 69, wherein the antibody or fusion protein exhibits cytotoxic activity of less than 10% cytolysis at an antibody or fusion protein concentration of at least 10 nM.

72. The recombinant polypeptide of any one of embodiments 59 to 71, wherein the antibody or fusion protein exhibits cytotoxic activity of less than 5% cytolysis at an antibody or fusion protein concentration of at least 10 nM.

73. The recombinant polypeptide of any one of embodiments 59 to 72, wherein the antibody or fusion protein exhibits cytotoxic activity of less than 4% cytolysis at an antibody or fusion protein concentration of at least 10 nM.

74. The recombinant polypeptide of any one of embodiments 59 to 73, wherein the antibody or fusion protein exhibits cytotoxic activity of less than 3% cytolysis at an antibody or fusion protein concentration of at least 10 nM.

75. The recombinant polypeptide of any one of embodiments 59 to 74, wherein the antibody or fusion protein exhibits cytotoxic activity of less than 2% cytolysis at an antibody or fusion protein concentration of at least 10 nM.

76. The recombinant polypeptide of any one of embodiments 59 to 75, wherein the antibody or fusion protein exhibits 0% cytolysis at an antibody or fusion protein concentration of at least 10 nM.

77. The recombinant polypeptide of any one of embodiments 59 to 69, wherein the antibody or fusion protein exhibits undetectable cytolysis at an antibody or fusion protein concentration of at least 10 nM.

78. The recombinant polypeptide of any one of embodiments 59 to 77, wherein the antibody or fusion protein exhibits increased expression in an expression system compared to an antibody comprising a wild type IgG1 constant domain comprising IgG1 hinge, IgG1 CH2, and IgG1 CH3.

79. The recombinant polypeptide of embodiment 78, wherein the expression system is a Chinese hamster ovary (CHO) stable expression system.

80. The recombinant polypeptide of embodiment 78 or embodiment 79, wherein the increase compared to the antibody or fusion protein comprising the wild type IgG1 constant domain is at least 5%.

81. The recombinant polypeptide of any one of embodiments 78 to 80, wherein the increase compared to the antibody or fusion protein comprising the wild type IgG1 constant domain is at least 10%.

82. The recombinant polypeptide of any one of embodiments 78 to 81, wherein the increase compared to the antibody or fusion protein comprising the wild type IgG1 constant domain is at least 15%.

83. The recombinant polypeptide of any one of embodiments 78 to 82, wherein the increase compared to the antibody or fusion protein comprising the wild type IgG1 constant domain is at least 20%.

84. The recombinant polypeptide of any one of embodiments 78 to 83, wherein the increase compared to the antibody or fusion protein comprising the wild type IgG1 constant domain is at least 25%.

85. The recombinant polypeptide of any one of embodiments 78 to 84, wherein the increase compared to the antibody or fusion protein comprising the wild type IgG1 constant domain is at least 30%.

86. The recombinant polypeptide of any one of embodiments 78 to 85, wherein the increase compared to the antibody or fusion protein comprising the wild type IgG1 constant domain is at least 40%

87. The recombinant polypeptide of any one of embodiments 78 to 86, wherein the increase compared to the antibody or fusion protein comprising the wild type IgG1 constant domain is at least 50%

88. The recombinant polypeptide of any one of embodiments 78 to 87, wherein the increase compared to the antibody or fusion protein comprising the wild type IgG1 constant domain is at least 60%

89. The recombinant polypeptide of any one of embodiments 78 to 88, wherein the increase compared to the antibody or fusion protein comprising the wild type IgG1 constant domain is at least 70%

90. The recombinant polypeptide of any one of embodiments 78 to 89, wherein the increase compared to the antibody or fusion protein comprising the wild type IgG1 constant domain is at least 80%

91. The recombinant polypeptide of any one of embodiments 78 to 90, wherein the increase compared to the antibody or fusion protein comprising the wild type IgG1 constant domain is at least 90%

92. The recombinant polypeptide of any one of embodiments 78 to 91, wherein the increase compared to the antibody or fusion protein comprising the wild type IgG1 constant domain is at least 100%.

93. The recombinant polypeptide of any one of embodiments 1 to 92, wherein the recombinant polypeptide exhibits at least 5-fold reduced binding to a human Fc receptor (FcR) than a polypeptide comprising a wild type IgG1 constant domain comprising IgG1 hinge, IgG1 CH2, and IgG1 CH3.

94. The recombinant polypeptide of any one of embodiments 1 to 93, wherein the recombinant polypeptide exhibits at least 10-fold reduced binding to a human Fc receptor (FcR) than a polypeptide comprising a wild type IgG1 constant domain comprising IgG1 hinge, IgG1 CH2, and IgG1 CH3.

95. The recombinant polypeptide of any one of embodiments 1 to 94, wherein the recombinant polypeptide exhibits at least 50-fold reduced binding to a human Fc receptor (FcR) than a polypeptide comprising a wild type IgG1 constant domain comprising IgG1 hinge, IgG1 CH2, and IgG1 CHS.

96. The recombinant polypeptide of any one of embodiments 1 to 95, wherein the recombinant polypeptide exhibits at least 100-fold reduced binding to a human Fc receptor (FcR) than a polypeptide comprising a wild type IgG1 constant domain comprising IgG1 hinge, IgG1CH2, and IgG1 CH3.

97. The recombinant polypeptide of any one of embodiments 1 to 96, wherein the recombinant polypeptide exhibits at least 500-fold reduced binding to a human Fc receptor (FcR) than a polypeptide comprising a wild type IgG1 constant domain comprising IgG1 hinge, IgG1 CH2, and IgG1 CH3.

98. The recombinant polypeptide of any one of embodiments 1 to 97, wherein the recombinant polypeptide exhibits at least 1,000-fold reduced binding to a human Fc receptor (FcR) than a polypeptide comprising a wild type IgG1 constant domain comprising IgG1 hinge, IgG1 CH2, and IgG1 CH3.

99. The recombinant polypeptide of any one of embodiments 1 to 98, wherein the recombinant polypeptide exhibits at least 5,000-fold reduced binding to a human Fc receptor (FcR) than a polypeptide comprising a wild type IgG1 constant domain comprising IgG1 hinge, IgG1 CH2, and IgG1 CH3.

100. The recombinant polypeptide of any one of embodiments 1 to 99, wherein the recombinant polypeptide exhibits at least 10,000-fold reduced binding to a human Fc receptor (FcR) than a polypeptide comprising a wild type IgG1 constant domain comprising IgG1 hinge, IgG1 CH2, and IgG1 CH3.

101. The recombinant polypeptide of any one of embodiments 93 to 100, wherein the FcR is FcRγ1.

102. The recombinant polypeptide of any one of embodiments 93 to 100, wherein the FcR is FcRγ2A.

103. The recombinant polypeptide of any one of embodiments 93 to 100, wherein the FcR is FcRγ2B.

104. The recombinant polypeptide of any one of embodiments 93 to 100, wherein the FcR is FcRγ3A.

105. The recombinant polypeptide of any one of embodiments 93 to 100, wherein the FcR is FcRγ3B.

106. A composition comprising a recombinant polypeptide of any one of embodiments 1 to 105.

107. The composition of embodiment 106, wherein the composition is a pharmaceutical composition comprising one or more excipients and/or pharmaceutically acceptable carriers.

108. A nucleic acid molecule or plurality of nucleic acid molecules encoding the recombinant polypeptide of any one of embodiments 1 to 105.

109. A host cell engineered to express the recombinant polypeptide of any one of embodiments 1 to 105 or the nucleic acid molecule(s) of embodiment 108.

110. A recombinant polypeptide comprising a heavy chain constant (CH) region comprising or consisting of an amino acid sequence at least 95% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously.

111. The recombinant polypeptide of embodiment 110, wherein the CH region comprises or consists of an amino acid sequence at least 96% identical to the amino acid sequence of any one of SEQ ID NO:3, SEQ ID NO:15, SEQ ID NO.16, SEQ ID NO:17, SEQ ID NO:18, SEQ ID NO:19, SEQ ID NO:20, SEQ ID NO:21, or SEQ ID NO:22.

112. The recombinant polypeptide of embodiment 110 or embodiment 111, wherein the CH region comprises or consists of an amino acid sequence at least 97% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

113. The recombinant polypeptide of any one of embodiments 110 to 112, wherein the CH region comprises or consists of an amino acid sequence at least 98% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

114. The recombinant polypeptide of any one of embodiments 110 to 113, wherein the CH region comprises or consists of an amino acid sequence at least 99% identical to the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

115. The recombinant polypeptide of any one of embodiments 110 to 114, wherein the CH region comprises or consists of the amino acid sequence of any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

116. The recombinant polypeptide of any one of embodiments 110 to 115, wherein the optional one or more CH2 domain amino acid substitutions reduce binding of the chimeric constant domain to an Fc receptor and/or reduces effector function.

117. The recombinant polypeptide any one of embodiments 110 to 116, wherein the optional one or more CH2 domain amino acid substitutions comprise an amino acid substitution at position G237 (EU numbering).

118. The recombinant polypeptide of any one of embodiments 110 to 117, wherein the optional one or more CH2 domain amino acid substitutions comprise an amino acid substitution at position D265 (EU numbering).

119. The recombinant polypeptide of embodiment 118, wherein the optional one or more CH2 domain amino acid substitutions comprise amino acid substitution D265A (EU numbering).

120. The recombinant polypeptide of any one of embodiments 110 to 119, wherein the optional one or more CH2 domain amino acid substitutions comprise an amino acid substitution at position N297 (EU numbering).

121. The recombinant polypeptide of embodiment 120, wherein the optional one or more CH2 domain amino acid substitutions comprise amino acid substitution N297A (EU numbering).

122. The recombinant polypeptide of embodiment 120, wherein the optional one or more CH2 domain amino acid substitutions comprise amino acid substitution N297D (EU numbering).

123. The recombinant polypeptide of any one of embodiments 110 to 122, wherein the optional one or more CH2 domain amino acid substitutions comprise an amino acid substitution at position P329 (EU numbering).

124. The recombinant polypeptide of embodiment 123, wherein the optional one or more CH2 domain amino acid substitutions comprise amino acid substitution P329A (EU numbering).

125. The recombinant polypeptide of embodiment 123, wherein the optional one or more CH2 domain amino acid substitutions comprise amino acid substitution P3290G (EU numbering).

126. The recombinant polypeptide of any one of embodiments 110 to 125, wherein the optional one or more CH2 domain amino acid substitutions comprise an amino acid substitution at position A330 (EU numbering).

127. The recombinant polypeptide of any one of embodiments 110 to 126, wherein the optional one or more CH2 domain amino acid substitutions comprise an amino acid substitution at position P331 (EU numbering).

128. The recombinant polypeptide of embodiment 127, wherein the optional one or more CH2 domain amino acid substitutions comprise amino acid substitution P331S (EU numbering).

129. The recombinant polypeptide of any one of embodiments 110 to 128, wherein the optional one or more CH2 domain amino acid substitutions comprise or consist of amino acid substitutions at positions D265 and N297 (EU numbering).

130. The recombinant polypeptide of embodiment 129, wherein the optional one or more CH2 domain amino acid substitutions comprise or consist of D265A and N297A (EU numbering).

131. The recombinant polypeptide of any one of embodiments 110 to 130, wherein the optional one or more CH3 domain amino acid substitutions provide for heterodimeric association of the chimeric constant domain with another constant domain.

132. The recombinant polypeptide of any one of embodiments 110 to 131, wherein the optional one or more CH3 domain amino acid substitutions comprise an amino acid substitution at position S354 or at E357 (EU numbering).

133. The recombinant polypeptide of embodiment 132, wherein the optional one or more CH3 domain amino acid substitutions comprise amino acid substitution S354C or E357C (EU numbering).

134. The recombinant polypeptide of any one of embodiments 110 to 133, wherein the optional one or more CH3 domain amino acid substitutions comprise an amino acid substitution at position T366 (EU numbering).

135. The recombinant polypeptide of embodiment 134, wherein the optional one or more CH3 domain amino acid substitutions comprise amino acid substitution T366W (EU numbering).

136. The recombinant polypeptide of any one of embodiments 110 to 135, wherein the optional one or more CH3 domain amino acid substitutions comprise or consist of amino acid substitutions at positions S354 and T366 (EU numbering).

137. The recombinant polypeptide of embodiment 136, wherein the optional one or more CH3 domain amino acid substitutions comprise or consist of S354C and T366W (EU numbering).

138. The recombinant polypeptide of any one of embodiments 110 to 137, wherein the optional one or more CH3 domain amino acid substitutions comprise an amino acid substitution at position Y349 (EU numbering).

139. The recombinant polypeptide of embodiment 138, wherein the optional one or more CH3 domain amino acid substitutions comprise amino acid substitution Y3490C (EU numbering).

140. The recombinant polypeptide of any one of embodiments 110 to 131, 138, or 139, wherein the optional one or more CH3 domain amino acid substitutions comprise an amino acid substitution at position Y407 (EU numbering).

141. The recombinant polypeptide of embodiment 140, wherein the optional one or more CH3 domain amino acid substitutions comprise amino acid substitution Y407V (EU numbering).

142. The recombinant polypeptide of embodiment 140 or embodiment 141, wherein the optional one or more CH3 domain amino acid substitutions comprise an amino acid substitution at position T366 (EU numbering).

143. The recombinant polypeptide of embodiment 142, wherein the optional one or more CH3 domain amino acid substitutions comprise amino acid substitution T366S (EU numbering).

144. The recombinant polypeptide of any one of embodiments 140 to 143, wherein the optional one or more CH3 domain amino acid substitutions comprise an amino acid substitution at position L368 (EU numbering).

145. The recombinant polypeptide of embodiment 144, wherein the optional one or more CH3 domain amino acid substitutions comprise amino acid substitution L368A (EU numbering).

146. The recombinant polypeptide of any one of embodiments 110 to 131, or 140 to 145, wherein the optional one or more CH3 domain amino acid substitutions comprise or consist of amino acid substitutions at positions Y349, T366, L363, and Y407 (EU numbering).

147. The recombinant polypeptide of embodiment 146, wherein the optional one or more CH3 domain amino acid substitutions comprise or consist of Y349C, T366S, L368A, and Y407V (EU numbering).

148. The recombinant polypeptide of any one of embodiments 110 to 147, wherein the optional one or more CH3 domain amino acid substitutions provides for selective purification of the recombinant polypeptide.

149. The recombinant polypeptide of embodiment 148, wherein the optional one or more CH3 domain amino acid substitutions comprise an amino acid substitution at position H435 (EU numbering).

150. The recombinant polypeptide of embodiment 149, wherein the optional one or more CH3 domain amino acid substitutions comprise amino acid substitution H435R (EU numbering).

151. The recombinant polypeptide of embodiment 148, wherein the optional one or more CH3 domain amino acid substitutions comprise an amino acid substitution at position Y436 (EU numbering).

152. The recombinant polypeptide of embodiment 151, wherein the optional one or more CH3 domain amino acid substitutions comprise amino acid substitution Y436F (EU numbering).

153. The recombinant polypeptide of any one of embodiments 110 to 152, wherein the recombinant polypeptide is an antigen-binding protein.

154. The recombinant polypeptide of any one of embodiments 110 to 153, further comprising an IgG1 CH1 domain or a fragment thereof.

155. The recombinant polypeptide of embodiment 154, wherein the fragment of IgG1 CH1 comprises or consists of DKKV (SEQ ID NO: 31).

156. The recombinant polypeptide of embodiment 154, wherein the fragment of IgG1 CH1 comprises or consists of DKRV (SEQ ID NO: 32).

157. The recombinant polypeptide of any one of embodiments 110 to 156, comprising at least one target binding domain.

158. The recombinant polypeptide of any one of embodiments 110 to 157, comprising at least two target binding domains.

159. The recombinant polypeptide of any one of embodiments 110 to 158, comprising at least three target binding domains.

160. The recombinant polypeptide of any one of embodiments 157 to 159, wherein the at least one target binding domain comprises a Fab.

161. The recombinant polypeptide of any one of embodiments 157 to 160, wherein the at least one target binding domain comprises an scFv.

162. The recombinant polypeptide of any one of embodiments 157 to 161, comprising a Fab as a first target binding domain and an scFv as a second target binding domain.

163. The recombinant polypeptide of any one of embodiments 157 to 160, comprising at least one target binding domain N-terminal to the chimeric constant domain.

164. The recombinant polypeptide of any one of embodiments 157 to 163, comprising at least one target binding domain C-terminal to the chimeric constant domain.

165. The recombinant polypeptide of any one of embodiments 157 to 164, comprising at least a first target binding domain N-terminal to the chimeric constant domain and at least a second target binding domain C-terminal to the chimeric constant domain.

166. The recombinant polypeptide of any one of embodiments 157 to 165, comprising from N- to C-terminus an scFv, a Fab, and the chimeric constant domain.

167. The recombinant polypeptide of any one of embodiments 110 to 166, wherein the recombinant polypeptide is or forms a part of an antibody, optionally a multispecific antibody.

168. The recombinant polypeptide of any one of embodiments 110 to 167, wherein the recombinant polypeptide is or forms a part of a monovalent antibody.

169. The recombinant polypeptide of any one of embodiments 110 to 167, wherein the recombinant polypeptide is or forms a part of a bivalent antibody.

170. The recombinant polypeptide of embodiment 169, wherein the recombinant polypeptide is or forms a part of a bispecific antibody.

171. The recombinant polypeptide of any one of embodiments 110 to 167, wherein the recombinant polypeptide is or forms a part of a trivalent antibody.

172. The recombinant polypeptide of embodiment 171, wherein the recombinant polypeptide is or forms a part of a bispecific antibody.

173. The recombinant polypeptide of embodiment 171, wherein the recombinant polypeptide is or forms a part of a trispecific antibody.

174. The recombinant polypeptide of any one of embodiments 110 to 167, wherein the recombinant polypeptide is or forms a part of a tetravalent antibody.

175. The recombinant polypeptide of embodiment 174, wherein the recombinant polypeptide is or forms a part of a bispecific antibody.

176. The recombinant polypeptide of embodiment 174, wherein the recombinant polypeptide is or forms a part of a trispecific antibody.

177. The recombinant polypeptide of any one of embodiments 110 to 157, wherein the recombinant polypeptide is or forms a part of a fusion protein.

178. The recombinant polypeptide of any one of embodiments 167 to 171, wherein the antibody or fusion protein exhibits cytotoxic activity of less than 20% cytolysis at an antibody or fusion protein concentration of at least 10 nM.

179. The recombinant polypeptide of any one of embodiments 167 to 178, wherein the antibody or fusion protein exhibits cytotoxic activity of less than 10% cytolysis at an antibody or fusion protein concentration of at least 10 nM.

180. The recombinant polypeptide of any one of embodiments 167 to 179, wherein the antibody or fusion protein exhibits cytotoxic activity of less than 5% cytolysis at an antibody or fusion protein concentration of at least 10 nM.

181. The recombinant polypeptide of any one of embodiments 167 to 180, wherein the antibody or fusion protein exhibits cytotoxic activity of less than 4% cytolysis at an antibody or fusion protein concentration of at least 10 nM.

182. The recombinant polypeptide of any one of embodiments 167 to 181, wherein the antibody or fusion protein exhibits cytotoxic activity of less than 3% cytolysis at an antibody or fusion protein concentration of at least 10 nM.

183. The recombinant polypeptide of any one of embodiments 167 to 182, wherein the antibody or fusion protein exhibits cytotoxic activity of less than 2% cytolysis at an antibody or fusion protein concentration of at least 10 nM.

184. The recombinant polypeptide of any one of embodiments 167 to 183, wherein the antibody or fusion protein exhibits 0% cytolysis at an antibody or fusion protein concentration of at least 10 nM.

185. The recombinant polypeptide of any one of embodiments 167 to 171, wherein the antibody or fusion protein exhibits undetectable cytolysis at an antibody or fusion protein concentration of at least 10 nM.

186. The recombinant polypeptide of any one of embodiments 167 to 185, wherein the antibody or fusion protein exhibits increased expression in an expression system compared to an antibody comprising a wild type IgG1 constant domain comprising IgG1 hinge, IgG1 CH2, and IgG1 CH3.

187. The recombinant polypeptide of embodiment 186, wherein the expression system is a Chinese hamster ovary (CHO) stable expression system.

188. The recombinant polypeptide of embodiment 186 or embodiment 187, wherein the increase compared to the antibody or fusion protein comprising the wild type IgG1 constant domain is at least 5%.

189. The recombinant polypeptide of any one of embodiments 186 to 188, wherein the increase compared to the antibody or fusion protein comprising the wild type IgG1 constant domain is at least 10%.

190. The recombinant polypeptide of any one of embodiments 186 to 189, wherein the increase compared to the antibody or fusion protein comprising the wild type IgG1 constant domain is at least 15%.

191. The recombinant polypeptide of any one of embodiments 186 to 190, wherein the increase compared to the antibody or fusion protein comprising the wild type IgG1 constant domain is at least 20%.

192. The recombinant polypeptide of any one of embodiments 186 to 191, wherein the increase compared to the antibody or fusion protein comprising the wild type IgG1 constant domain is at least 25%.

193. The recombinant polypeptide of any one of embodiments 186 to 192, wherein the increase compared to the antibody or fusion protein comprising the wild type IgG1 constant domain is at least 30%.

194. The recombinant polypeptide of any one of embodiments 186 to 193, wherein the increase compared to the antibody or fusion protein comprising the wild type IgG1 constant domain is at least 40%

195. The recombinant polypeptide of any one of embodiments 186 to 194, wherein the increase compared to the antibody or fusion protein comprising the wild type IgG1 constant domain is at least 50%

196. The recombinant polypeptide of any one of embodiments 186 to 195, wherein the increase compared to the antibody or fusion protein comprising the wild type IgG1 constant domain is at least 60%

197. The recombinant polypeptide of any one of embodiments 186 to 196, wherein the increase compared to the antibody or fusion protein comprising the wild type IgG1 constant domain is at least 70%

198. The recombinant polypeptide of any one of embodiments 186 to 197, wherein the increase compared to the antibody or fusion protein comprising the wild type IgG1 constant domain is at least 80%

199. The recombinant polypeptide of any one of embodiments 186 to 198, wherein the increase compared to the antibody or fusion protein comprising the wild type IgG1 constant domain is at least 90%

200. The recombinant polypeptide of any one of embodiments 186 to 199, wherein the increase compared to the antibody or fusion protein comprising the wild type IgG1 constant domain is at least 100%.

201. The recombinant polypeptide of any one of embodiments 110 to 200, wherein the recombinant polypeptide exhibits at least 5-fold reduced binding to a human Fc receptor (FcR) than a polypeptide comprising a wild type IgG1 constant domain comprising IgG1 hinge, IgG1 CH2, and IgG1 CH3.

202. The recombinant polypeptide of any one of embodiments 110 to 201, wherein the recombinant polypeptide exhibits at least 10-fold reduced binding to a human Fc receptor (FcR) than a polypeptide comprising a wild type IgG1 constant domain comprising IgG1 hinge, IgG1 CH2, and IgG1 CH.

203. The recombinant polypeptide of any one of embodiments 110 to 202, wherein the recombinant polypeptide exhibits at least 50-fold reduced binding to a human Fc receptor (FcR) than a polypeptide comprising a wild type IgG1 constant domain comprising IgG1 hinge, IgG1 CH2, and IgG1 CHS.

204. The recombinant polypeptide of any one of embodiments 110 to 203, wherein the recombinant polypeptide exhibits at least 100-fold reduced binding to a human Fc receptor (FcR) than a polypeptide comprising a wild type IgG1 constant domain comprising IgG1 hinge, gG1CH2, and IgG1 CH3.

205. The recombinant polypeptide of any one of embodiments 110 to 204, wherein the recombinant polypeptide exhibits at least 500-fold reduced binding to a human Fc receptor (FcR) than a polypeptide comprising a wild type IgG1 constant domain comprising IgG1 hinge, IgG1 CH2, and IgG1 CH3.

206. The recombinant polypeptide of any one of embodiments 110 to 205, wherein the recombinant polypeptide exhibits at least 1,000-fold reduced binding to a human Fc receptor (FcR) than a polypeptide comprising a wild type IgG1 constant domain comprising IgG1 hinge, IgG1 CH2, and IgG1 CHS.

207. The recombinant polypeptide of any one of embodiments 110 to 206, wherein the recombinant polypeptide exhibits at least 5,000-fold reduced binding to a human Fc receptor (FcR) than a polypeptide comprising a wild type IgG1 constant domain comprising IgG1 hinge, IgG1 CH2, and IgG1 CHS.

208. The recombinant polypeptide of any one of embodiments 110 to 207, wherein the recombinant polypeptide exhibits at least 10,000-fold reduced binding to a human Fc receptor (FcR) than a polypeptide comprising a wild type IgG1 constant domain comprising IgG1 hinge, IgG1 CH2, and IgG1 CH3.

209. The recombinant polypeptide of any one of embodiments 201 to 208, wherein the FcR is FcRγ1.

210. The recombinant polypeptide of any one of embodiments 201 to 208, wherein the FcR is FcRγ2A.

211. The recombinant polypeptide of any one of embodiments 201 to 208, wherein the FcR is FcRγ2B.

212. The recombinant polypeptide of any one of embodiments 201 to 208, wherein the FcR is FcRγ3A.

213. The recombinant polypeptide of any one of embodiments 201 to 208, wherein the FcR is FcRγ3B.

214. A composition comprising a recombinant polypeptide of any one of embodiments 110 to 213.

215. The composition of embodiment 214, wherein the composition is a pharmaceutical composition comprising one or more excipients and/or pharmaceutically acceptable carriers.

216. A nucleic acid molecule or plurality of nucleic acid molecules encoding the recombinant polypeptide of any one of embodiments 110 to 215.

217. A host cell engineered to express the recombinant polypeptide of any one of embodiments 110 to 213 or the nucleic acid molecule(s) of embodiment 216.

218. A method of producing recombinant polypeptide comprising a chimeric constant domain, comprising culturing the host cell of claim 109 or claim 217 under conditions in which the recombinant polypeptide is expressed.

219. The method of claim 218, further comprising recovering and, optionally, purifying the recombinant polypeptide.

### Group B Numbered Embodiments

1. A method for increasing production of a fusion protein comprising a constant domain, comprising expressing the polypeptide with a chimeric constant domain comprising, from N-terminus to C-terminus:
(a) a chimeric immunoglobulin hinge comprising:
   (i) a human IgG1 upper hinge amino acid sequence EPKSCDKTHT (SEQ ID NO: 24) from positions 216 to 225 (EU numbering)
   (ii) a human IgG1 core hinge amino acid sequence CPPC (SEQ ID NO: 25) from positions 226 to 229 (EU numbering);
   (iii) a human IgG2 lower hinge amino acid sequence PCPAPPVAGP (SEQ ID NO: 42) from positions 230 to 238 (EU numbering);
(b) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously; and
(c) a CH3 domain comprising a human IgG1 CH3 domain amino acid sequence from position 341 to 447 (EU numbering) of human IgG1, optionally comprising one or more CH3 domain amino acid substitutions, optionally wherein the substitutions are selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing.

2. The method of embodiment 1, wherein the method increases expression relative to expression of a variant fusion protein comprising a control constant domain.

3. The method of embodiment 2, wherein the control constant domain is a constant domain of SEQ ID NO:1 with or without mutations permitting for heterodimerization or purification, e.g., knob-in-hole mutations and/or star mutations.

4. The method of embodiment 2, wherein the control constant domain is a constant domain of SEQ ID NO:9 with or without mutations permitting for heterodimerization or purification, e.g., knob-in-hole mutations and/or star mutations.

5. The method of embodiment 2, wherein the control constant domain is a constant domain of SEQ ID NO:10 with or without mutations permitting for heterodimerization or purification, e.g., knob-in-hole mutations and/or star mutations.

6. The method of any one of embodiments 2 to 5, wherein the increase in expression relative to the variant fusion protein is at least 10%.

7. The method of any one of embodiments 2 to 5, wherein the increase in expression relative to the variant fusion protein is at least 15%.

8. The method of any one of embodiments 2 to 5, wherein the increase in expression relative to the variant fusion protein is at least 20%.

9. The method of any one of embodiments 2 to 5, wherein the increase in expression relative to the variant fusion protein is at least 30%.

10. The method of any one of embodiments 2 to 5, wherein the increase in expression relative to the variant fusion protein is at least 40%.

11. The method of any one of embodiments 2 to 5, wherein the increase in expression relative to the variant fusion protein is at least 50%.

12. A method for increasing activity of a fusion protein with a constant domain, comprising expressing the polypeptide with a chimeric constant domain comprising, from N-terminus to C-terminus:
(a) a chimeric immunoglobulin hinge comprising:
   (i) a human IgG1 upper hinge amino acid sequence EPKSCDKTHT (SEQ ID NO: 24) from positions 216 to 225 (EU numbering)
   (ii) a human IgG1 core hinge amino acid sequence CPPC (SEQ ID NO: 25) from positions 226 to 229 (EU numbering);
   (iii) a human IgG2 lower hinge amino acid sequence PCPAPPVAGP (SEQ ID NO: 42) from positions 230 to 238 (EU numbering);
(b) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously; and
(c) a CH3 domain comprising a human IgG1 CH3 domain amino acid sequence from position 341 to 447 (EU numbering) of human IgG1, optionally comprising one or more CH3 domain amino acid substitutions, optionally wherein the substitutions are selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing.

13. The method of embodiment 6, wherein the method increases expression relative to expression of a variant fusion protein comprising a control constant domain.

14. The method of embodiment 13, wherein the control constant domain is a constant domain of SEQ ID NO:1 with or without mutations permitting for heterodimerization or purification, e.g., knob-in-hole mutations and/or star mutations.

15. The method of embodiment 13, wherein the control constant domain is a constant domain of SEQ ID NO:9 with or without mutations permitting for heterodimerization or purification, e.g., knob-in-hole mutations and/or star mutations.

16. The method of embodiment 13, wherein the control constant domain is a constant domain of SEQ ID NO:10 with or without mutations permitting for heterodimerization or purification, e.g., knob-in-hole mutations and/or star mutations.

17. The method of any one of embodiments 13 to 16, wherein the increase in activity relative to the variant fusion protein is at least 10%.

18. The method of any one of embodiments 13 to 16, wherein the increase in activity relative to the variant fusion protein is at least 15%.

19. The method of any one of embodiments 13 to 16, wherein the increase in activity relative to the variant fusion protein is at least 20%.

20. The method of any one of embodiments 13 to 16, wherein the increase in activity relative to the variant fusion protein is at least 30%.

21. The method of any one of embodiments 13 to 16, wherein the increase in activity relative to the variant fusion protein is at least 40%.

22. The method of any one of embodiments 13 to 16, wherein the increase in activity relative to the variant fusion protein is at least 50%.

23. A method for increasing expression and activity of a fusion protein with a constant domain, comprising expressing the polypeptide with a chimeric constant domain comprising, from N-terminus to C-terminus:
(a) a chimeric immunoglobulin hinge comprising:
   (i) a human IgG1 upper hinge amino acid sequence EPKSCDKTHT (SEQ ID NO: 24) from positions 216 to 225 (EU numbering)
   (ii) a human IgG1 core hinge amino acid sequence CPPC (SEQ ID NO: 25) from positions 226 to 229 (EU numbering);
   (iii) a human IgG2 lower hinge amino acid sequence PCPAPPVAGP (SEQ ID NO: 42) from positions 230 to 238 (EU numbering);:
(b) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously; and
(c) a CH3 domain comprising a human IgG1 CH3 domain amino acid sequence from position 341 to 447 (EU numbering) of human IgG1, optionally comprising one or more CH3 domain amino acid substitutions, optionally wherein the substitutions are selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing.

24. The method of embodiment 23, wherein the method increases expression and activity relative to expression of a variant fusion protein comprising a control constant domain.

25. The method of embodiment 24, wherein the control constant domain is a constant domain of SEQ ID NO:1 with or without mutations permitting for heterodimerization or purification, e.g., knob-in-hole mutations and/or star mutations.

26. The method of embodiment 24, wherein the control constant domain is a constant domain of SEQ ID NO:9 with or without mutations permitting for heterodimerization or purification, e.g., knob-in-hole mutations and/or star mutations.

27. The method of embodiment 24, wherein the control constant domain is a constant domain of SEQ ID NO:10 with or without mutations permitting for heterodimerization or purification, e.g., knob-in-hole mutations and/or star mutations.

28. The method of any one of embodiments 24 to 27, wherein:
(a) the increase in expression relative to the variant fusion protein is at least 10%; and
(b) the increase in activity relative to the variant fusion protein is at least 10%.

29. The method of embodiment 28, wherein the increase in expression relative to the variant fusion polypeptide is at least 15%.

30. The method of embodiment 28, wherein the increase in expression relative to the variant fusion polypeptide is at least 20%.

31. The method of embodiment 28, wherein the increase in expression relative to the variant fusion polypeptide is at least 30%.

32. The method of embodiment 28, wherein the increase in expression relative to the variant fusion polypeptide is at least 40%.

33. The method of embodiment 28, wherein the increase in expression relative to the variant fusion polypeptide is at least 50%.

34. The method of any one of embodiments 28 to 33, wherein the increase in activity relative to the variant fusion polypeptide is at least 15%.

35. The method of any one of embodiments 28 to 33, wherein the increase in activity relative to the variant fusion polypeptide is at least 20%.

36. The method of any one of embodiments 28 to 33, wherein the increase in activity relative to the variant fusion polypeptide is at least 30%.

37. The method of any one of embodiments 23 to 33, wherein the increase in activity relative to the variant fusion polypeptide is at least 40%.

38. The method of any one of embodiments 28 to 33, wherein the increase in activity relative to the variant fusion polypeptide is at least 50%.

39. The method of any one of embodiments 1 to 38, wherein the optional one or more CH2 domain amino acid substitutions reduces binding of the chimeric constant domain to an Fc receptor and/or reduces effector function.

40. The method of any one of embodiments 1 to 39, wherein the optional one or more CH3 domain amino acid substitutions provides for heterodimeric association of the chimeric constant domain with another constant domain.

41. The method of any one of embodiments 1 to 40, wherein the optional one or more CH3 domain amino acid substitutions provides for selective purification of the fusion protein.

42. The method of any one of embodiments 1 to 41, wherein the fusion protein is an antigen-binding protein.

43. The method of any one of embodiments 1 to 42, wherein the chimeric constant domain comprises an IgG1 CH1 domain or a fragment thereof, optionally wherein the fragment of IgG1 CH1 comprises or consists of DKKV (SEQ ID NO: 31) or DKRV (SEQ ID NO: 32).

44. The method of any one of embodiments 1 to 43, wherein the fusion protein comprises at least one target binding domain.

45. The method of any one of embodiments 1 to 44, wherein the fusion protein comprises at least two target binding domains.

46. The method of any one of embodiments 1 to 45, wherein the fusion protein comprises at least three target binding domains.

47. The method of any one of embodiments 1 to 46, wherein the fusion protein comprises at least one Fab.

48. The method of any one of embodiments 1 to 47, wherein the fusion protein comprises at least one scFv.

49. The method of any one of embodiments 1 to 48, wherein the fusion protein comprises at least one Fab and at least one scFv.

50. The method of any one of embodiments 1 to 49, wherein the fusion protein comprises two Fabs and one scFv.

51. The method of any one of embodiments 1 to 50, wherein the fusion protein comprises at least one target binding domain N-terminal to the chimeric constant domain.

52. The method of any one of embodiments 1 to 51, wherein the fusion protein comprises at least one target binding domain C-terminal to the chimeric constant domain.

53. The method of any one of embodiments 1 to 52, wherein the fusion protein comprises a first target binding domain N-terminal to the chimeric constant domain and at least a second target binding domain C-terminal to the chimeric constant domain.

54. The method of any one of embodiments 1 to 53, wherein the fusion protein comprises from N- to C-terminus an scFv, a Fab, and the chimeric constant domain.

55. The method of any one of embodiments 1 to 54, wherein the fusion protein is or forms a part of an antibody, optionally a multispecific antibody.

56. The method of any one of embodiments 1 to 55, wherein the fusion protein is a dimer comprising two chimeric constant domains as defined in any one of embodiments 1 and 39 to 41, optionally wherein each chimeric constant domain is independently selected from a constant domain having the amino acid sequence of any one of SEQ ID NOs:3, 15, 16, 17, 18, 19, 20, 21, 22.

57. The method of embodiment 56, wherein the fusion protein is a homodimer.

58. The method of embodiment 56, wherein the fusion protein is a heterodimer.

59. The method of embodiment 58, in which one of the chimeric constant domains comprises knob substitutions and the other chimeric constant domain comprises hole substitutions.

60. The method of embodiment 58 or embodiment 59, in which the chimeric constant domains comprise star mutations.

61. The method of any one of embodiments 1 to 60, wherein the fusion protein comprises two polypeptide chains.

62. The method of any one of embodiments 1 to 60, wherein the fusion protein comprises three polypeptide chains.

63. The method of any one of embodiments 1 to 60, wherein the fusion protein comprises four polypeptide chains.

64. The method of any one of embodiments 1 to 63, wherein expressing the fusion protein comprises culturing a host cell engineered to express the fusion protein under conditions in which the fusion protein is expressed.

65. The method of embodiment 64, wherein the host cell is a mammalian host cell.

66. The method of embodiment 64 or embodiment 65, which further comprises recovering and, optionally, purifying the expressed fusion protein.

67. The method of any one of embodiments 64 to 66, wherein the host cell is a Chinese hamster ovary (CHO) cell or an HEK293 or derivative (e.g., Expi293F) cell.

68. The method of any one of embodiments 1 to 67, which further comprises, prior to said expressing step, introducing into a host cell a nucleic acid molecule or a plurality of nucleic acid molecules encoding the fusion protein.

69. The method of embodiment 64, wherein the nucleic acid molecule or a plurality of nucleic acid molecules are included in one or more host cell expression vectors.

70. A population of fusion proteins produced by the method of any one of embodiments 1 to 69.

71. The population of embodiment 70, comprising at least 10,000 fusion proteins.

72. The population of embodiment 70 or embodiment 71, which is characterized by greater activity as compared to a population of fusion proteins comprising a control constant domain comprising the amino acid sequence of SEQ ID NO:1, SEQ ID NO:9, or SEQ ID NO:10 with or without mutations permitting for heterodimerization or purification, e.g., knob-in-hole mutations and/or star mutations).

### Group C Numbered Embodiments

1. A fusion protein comprising a first polypeptide chain associated with a second polypeptide chain, wherein:
(a) the first polypeptide chain comprises a first chimeric constant domain comprising an amino acid sequence that has at least 95% identity to SEQ ID NO:1, provided that chimeric constant domain has:
   (i) the sequence P-V-A-absent at amino acids 233 to 236 as defined by EU numbering;
   (ii) one or more amino acid substitutions that promote heterodimerization with the second polypeptide chain; and
(b) the second polypeptide chain comprises a second chimeric constant domain comprising an amino acid sequence that has at least 95% identity to SEQ ID NO:1, provided that chimeric constant domain has:
   (i) the sequence P-V-A-absent at amino acids 233 to 236 as defined by EU numbering; and
   (ii) one or more amino acid substitutions that promote heterodimerization with the first polypeptide chain.

2. A fusion protein comprising a first polypeptide chain associated with a second polypeptide chain, wherein:
(a) the first polypeptide chain comprises a first chimeric constant domain comprising, from N-terminus to C-terminus:
   (i) a chimeric immUnoglobulin hinge comprising:
      (1) a human IgG1 upper hinge amino acid sequence EPKSCDKTHT (SEQ ID NO: 24) from positions 216 to 225 (EU numbering)
      (2) a human IgG1 core hinge amino acid sequence CPPC (SEQ ID NO: 25) from positions 226 to 229 (EU numbering);
      (3) a human IgG2 lower hinge amino acid sequence PCPAPPVAGP (SEQ ID NO: 42) from positions 230 to 238 (EU numbering);
   (ii) a CH2 domain comprising a human IgG1 CH2 domain amino acid sequence from position 237 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously;
   (iii) a CH3 domain comprising a human IgG1 CH3 domain amino acid sequence from position 341 to 447 (EU numbering) of human IgG1;
   (iv) one or more amino acid substitutions that promote heterodimerization with the second polypeptide chain; and
   (v) optionally, one or more amino acid substitutions to reduce effector function and/or to facilitate purification; and
(b) the second polypeptide chain comprises a second chimeric constant domain comprising, from N-terminus to C-terminus:
   (i) a chimeric immunoglobulin hinge comprising a human IgG1 upper hinge amino acid sequence EPKSCDKTHTCP (SEQ ID NO: 43) from positions 216 to 227 (EU numbering) of human IgG1 and a human IgG2 lower hinge amino acid sequence PCPAPPVA (SEQ ID NO: 44) from positions 228 to 236 (EU numbering) of human IgG2;
   (ii) a CH2 domain comprising a human IgG1 CH2 domain amino acid sequence from position 237 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously;
   (iii) a CH3 domain comprising a human IgG1 CH3 domain amino acid sequence from position 341 to 447 (EU numbering) of human IgG1;
   (iv) one or more amino acid substitutions that promote heterodimerization with the first polypeptide chain; and
(c) optionally, one or more amino acid substitutions to reduce effector function and/or to facilitate purification.

3. The fusion protein of embodiment 2, wherein the first chimeric constant domain and second chimeric constant domain each comprises or consists of an amino acid sequence having at least 95% sequence identity to the amino acid sequence of SEQ ID NO:1.

4. The fusion protein of embodiment 1 or embodiment 2, wherein the first chimeric constant domain and second chimeric constant domain each comprises or consists of an amino acid sequence having at least 96% sequence identity to the amino acid sequence of SEQ ID NO:1.

5. The fusion protein of embodiment 1 or embodiment 2, wherein the first chimeric constant domain and second chimeric constant domain each comprises or consists of an amino acid sequence having at least 97% sequence identity to the amino acid sequence of SEQ ID NO:1.

6. The fusion protein of embodiment 1 or embodiment 2, wherein the first chimeric constant domain and second chimeric constant domain each comprises or consists of an amino acid sequence having at least 98% sequence identity to the amino acid sequence of SEQ ID NO:1.

7. The fusion protein of embodiment 1 or embodiment 2 wherein the first chimeric constant domain and second chimeric constant domain each comprises or consists of an amino acid sequence having at least 98% sequence identity to the amino acid sequence of SEQ ID NO:1.

8. The fusion protein of any one of embodiments 1 to 7, wherein first chimeric constant domain comprises the amino acid sequence of SEQ ID NO:15 and wherein first chimeric constant domain comprises the amino acid sequence of SEQ ID NO:16.

9. The fusion protein of any one of embodiments 1 to 7, wherein first chimeric constant domain comprises the amino acid sequence of SEQ ID NO:17 and wherein first chimeric constant domain comprises the amino acid sequence of SEQ ID NO:16.

10. The fusion protein of any one of embodiments 1 to 7, wherein first chimeric constant domain comprises the amino acid sequence of SEQ ID NO:15 and wherein first chimeric constant domain comprises the amino acid sequence of SEQ ID NO:18.

11. The fusion protein of any one of embodiments 1 to 7, wherein first chimeric constant domain comprises the amino acid sequence of SEQ ID NO:17 and wherein first chimeric constant domain comprises the amino acid sequence of SEQ ID NO:18.

12. The fusion protein of any one of embodiments 1 to 7, wherein first chimeric constant domain comprises the amino acid sequence of SEQ ID NO:19 and wherein first chimeric constant domain comprises the amino acid sequence of SEQ ID NO:20.

13. The fusion protein of any one of embodiments 1 to 7, wherein first chimeric constant domain comprises the amino acid sequence of SEQ ID NO:19 and wherein first chimeric constant domain comprises the amino acid sequence of SEQ ID NO:22.

14. The fusion protein of any one of embodiments 1 to 7, wherein first chimeric constant domain comprises the amino acid sequence of SEQ ID NO:21 and wherein first chimeric constant domain comprises the amino acid sequence of SEQ ID NO:20.

15. The fusion protein of any one of embodiments 1 to 7, wherein first chimeric constant domain comprises the amino acid sequence of SEQ ID NO:21 and wherein first chimeric constant domain comprises the amino acid sequence of SEQ ID NO:22.

16. The fusion protein any one of embodiments 1 to 15, wherein the fusion protein is an antigen-binding protein comprising at least one target-binding domain.

17. The fusion protein any one of embodiments 1 to 16, wherein the fusion protein is or forms a part of an antibody, optionally a multispecific antibody.

18. The fusion protein of embodiment 16 or embodiment 17, comprising at least two target binding domains.

19. The fusion protein of embodiment 18, wherein the fusion protein is or forms a part of a bivalent antibody.

20. The fusion protein of any one of embodiments 16 to 18, comprising at least three target binding domains.

21. The fusion protein of embodiment 20, wherein the fusion protein is or forms a part of a trivalent antibody.

22. The fusion protein of any one of embodiments 16 to 21, wherein at least one target binding domain comprises a Fab.

23. The fusion protein of any one of embodiments 16 to 21, wherein at least one target binding domain comprises an scFv.

24. The fusion protein of any one of embodiments 16 to 21, comprising a Fab as a first target binding domain and an scFv as a second target binding domain.

25. The fusion protein embodiment 20 or embodiment 21, comprising a Fab as a first target binding domain, an scFv as a second target binding domain, and a Fab as a third target binding domain.

26. The fusion protein of any one of embodiments 16 to 25, which comprises a third polypeptide chain and a fourth polypeptide chain, wherein:
(a) the first polypeptide chain comprises, from N-terminus to C-terminus a first heavy chain variable region (VH1), a CH1 domain, and the first constant domain; and
(b) the second polypeptide chain comprises, from N-terminus to C-terminus, a second heavy chain variable region (VH2), a CH1 domain, and the second constant domain;
(c) the third polypeptide chain comprises first light chain comprising a first light chain variable region (VL1) associated with VH1 to form a first Fab domain; and
(d) the fourth polypeptide chain comprises first light chain comprising a second light chain variable region (VL2) associated with VH2 to form a second Fab domain.

27. The fusion protein of embodiment 26, wherein VH1 and VH2 are identical.

28. The fusion protein of embodiment 26, wherein VH1 and VH2 are different.

29. The fusion protein of embodiment 28, wherein the first Fab domain and second Fab domain bind to different epitopes on the same target molecule.

30. The fusion protein of embodiment 28, wherein the first Fab domain and second Fab domain bind to different target molecules.

31. The fusion protein of embodiment 20 or embodiment 21, which comprises a third polypeptide chain and a fourth polypeptide chain, wherein:
(a) the first polypeptide chain comprises, from N-terminus to C-terminus an scFv, an optional linker, a first heavy chain variable region (VH1), a CH1 domain, and the first constant domain; and
(b) the second polypeptide chain comprises, from N-terminus to C-terminus, a second heavy chain variable region (VH2), a CH1 domain, and the second constant domain, optionally wherein VH2 is identical to VH1;
(c) the third polypeptide chain comprises first light chain comprising a first light chain variable region (VL1) associated with VH1 to form a first Fab domain; and
(d) the fourth polypeptide chain comprises first light chain comprising a second light chain variable region (VL2) associated with VH2 to form a second Fab domain.

32. The fusion protein of embodiment 20 or embodiment 21, which comprises a third polypeptide chain and a fourth polypeptide chain, wherein:
(a) the first polypeptide chain comprises, from N-terminus to C-terminus, a first heavy chain variable region (VH1), a CH1 domain, and the first constant domain; and
(b) the second polypeptide chain comprises, from N-terminus to C-terminus, an scFv, an optional linker, a second heavy chain variable region (VH2), a CH1 domain, and the second constant domain, optionally wherein VH2 is identical to VH1;
(c) the third polypeptide chain comprises first light chain comprising a first light chain variable region (VL1) associated with VH1 to form a first Fab domain; and
(d) the fourth polypeptide chain comprises first light chain comprising a second light chain variable region (VL2) associated with VH2 to form a second Fab domain.

33. The fusion protein of embodiment 31 or embodiment 32, wherein the scFv comprises a third heavy chain variable region (VH2) and a third light chain variable region (VL3) arranged, from N-terminus to C-terminus, VH3-optional linker-VL3.

34. The fusion protein of embodiment 31 or embodiment 32, wherein the scFv comprises a third heavy chain variable region (VH2) and a third light chain variable region (VL3) arranged, from N-terminus to C-terminus, VL3-optional linker-VH3.

35. The fusion protein of any one of embodiments 31 to 34, wherein the scFv binds to a different target molecule than the first Fab domain and/or the second Fab domain.

36. The fusion protein of any one of embodiments 1 to 35, which exhibits at least 10% increased expression in an expression system compared to a variant protein comprising:
(a) the sequence E-L-L-G (SEQ ID NO: 23) at amino acids 233 to 236 as defined by EU numbering in its first and second chimeric constant domains; and/or
(b) first and second chimeric constant domains having the amino acid sequence of SEQ ID NO:10, with or without mutations permitting for heterodimerization or purification, e.g., knob-in-hole mutations and/or star mutations.

37. The fusion protein of embodiment 36, wherein the expression system is a Chinese hamster ovary (CHO) expression system or an HEK293 or derivative (e.g., Expi293F) expression system.

38. The fusion protein of embodiment 36 or embodiment 37, wherein the increase in expression is at least 20% or at least 30%.

39. The fusion protein of any one of embodiments 1 to 38 which exhibits at least 5-fold reduced binding to a human Fc receptor (FcR) compared to a variant protein comprising:
(a) the sequence E-L-L-G (SEQ ID NO: 23) at amino acids 233 to 236 as defined by EU numbering in its first and second chimeric constant domains; and/or
(b) first and second chimeric constant domains having the amino acid sequence of SEQ ID NO:9, with or without mutations permitting for heterodimerization or purification, e.g., knob-in-hole mutations and/or star mutations.

40. The fusion protein of embodiment 39, wherein the reduction in binding is at least 10-fold or at least 20-fold.

41. The fusion protein of embodiment 39 or embodiment 40, wherein the FcR is FcRγ1.

42. The fusion protein of embodiment 39 or embodiment 40, wherein the FcR is FcRγ2A.

43. The fusion protein of embodiment 39 or embodiment 40, wherein the FcR is FcRγ2B.

44. The fusion protein of embodiment 39 or embodiment 40, wherein the FcR is FcRγ3A.

45. The fusion protein of embodiment 39 or embodiment 40, wherein the FcR is FcRγ3B.

46. A composition comprising the fusion protein of any one of embodiments 1 to 45.

47. The composition of embodiment 46, wherein the composition is a pharmaceutical composition comprising one or more excipients and/or pharmaceutically acceptable carriers.

48. A nucleic acid molecule or plurality of nucleic acid molecules encoding the fusion protein of any one of embodiments 1 to 45.

49. A host cell engineered to express the fusion protein of any one of embodiments 1 to 45.

50. A host cell comprising one or more expression vectors encoding the fusion protein of any one of embodiments 1 to 45.

51. The host cell of embodiment 49 or embodiment 50 which is a CHO cell or an HEK293 or derivative (e.g., Expi293F) cell.

52. A method of producing the fusion protein of any one of embodiments 1 to 45, comprising culturing the host cell of any one of embodiments 49 to 51 and optionally recovering and/or purifying the expressed protein.

53. A population of fusion proteins according to any one of embodiments 1 to 45.

54. The population of fusion proteins of embodiment 53 which is produced by the method of any one of embodiments 52.

55. The population of embodiment 53 or embodiment 54, comprising at least 10,000 fusion proteins.

### Group D Numbered Embodiments

1. A recombinant protein comprising a constant domain having:
(a) the amino acid sequence of SEQ ID NO:3;
(b) the amino acid sequence of SEQ ID NO: 15;
(c) the amino acid sequence of SEQ ID NO:16;
(d) the amino acid sequence of SEQ ID NO:17;
(e) the amino acid sequence of SEQ ID NO:18;
(f) the amino acid sequence of SEQ ID NO: 19;
(g) the amino acid sequence of SEQ ID NO:20
(h) the amino acid sequence of SEQ ID NO:21;
(i) the amino acid sequence of SEQ ID NO:22;
(j) an amino acid sequence that has at least 97%, 98%, 99% or 100% sequence identity to any one of SEQ ID NO:3, SEQ ID NO:15, SEQ ID NO:16, SEQ ID NO:17, or SEQ ID NO:18, provided that the Fc domain (i) has the sequence P-V-A-absent at amino acids 233 to 236 as defined by EU numbering and (ii) optionally has (1) a star mutation and/or (2) a knob or hole mutation and/or (3) a S3540 or E357C mutation (as defined by EU numbering).

2. The recombinant protein of embodiment 1, which is a multimer.

3. The recombinant protein of embodiment 2 which comprises two polypeptide chains.

4. The recombinant protein of embodiment 2 which comprise three polypeptide chains.

5. The recombinant protein of embodiment 2 which comprises four polypeptide chains.

6. The recombinant protein of any one of embodiments 1 to 5, which comprises an Fc homodimer.

7. The recombinant protein of embodiment 6, which comprises two constant domains, each having the amino acid sequence of SEQ ID NO:3.

8. The recombinant protein of embodiment 6, which comprises two identical constant domains, each having an amino acid sequence that has at least 97% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22, provided that the Fc domain has the sequence P-V-A-absent at amino acids 233 to 236.

9. The recombinant protein of embodiment 6, which comprises two identical constant domains, each having an amino acid sequence that has at least 98% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22, provided that the Fc domain has the sequence P-V-A-absent at amino acids 233 to 236.

10. The recombinant protein of embodiment 6, which comprises two identical constant domains, each having an amino acid sequence that has at least 99% to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22, provided that the Fc domain has the sequence P-V-A-absent at amino acids 233 to 236.

11. The recombinant protein of embodiment 6, which comprises two identical constant domains, each having an amino acid sequence that has at 100% sequence identity to SEQ ID NO:3.

12. The recombinant protein of any one of embodiments 1 to 5, which comprises an Fc heterodimer.

13. The recombinant protein of embodiment 12, which comprises a first constant domain having the amino acid sequence of SEQ ID NO:15 and a second constant domain having the amino acid sequence of SEQ ID NO: 16.

14. The recombinant protein of embodiment 12, which comprises a first constant domain having the amino acid sequence of SEQ ID NO:15 and a second constant domain having the amino acid sequence of SEQ ID NO: 18.

15. The recombinant protein of embodiment 12, which comprises a first constant domain having the amino acid sequence of SEQ ID NO:17 and a second constant domain having the amino acid sequence of SEQ ID NO: 16.

16. The recombinant protein of embodiment 12, which comprises a first constant domain having the amino acid sequence of SEQ ID NO:17 and a second constant domain having the amino acid sequence of SEQ ID NO: 18.

17. The recombinant protein of embodiment 12, which comprises a first constant domain having the amino acid sequence of SEQ ID NO:19 and a second constant domain having the amino acid sequence of SEQ ID NO:20.

18. The recombinant protein of embodiment 12, which comprises a first constant domain having the amino acid sequence of SEQ ID NO:19 and a second constant domain having the amino acid sequence of SEQ ID NO:22.

19. The recombinant protein of embodiment 12, which comprises a first constant domain having the amino acid sequence of SEQ ID NO:21 and a second constant domain having the amino acid sequence of SEQ ID NO:20.

20. The recombinant protein of embodiment 12, which comprises a first constant domain having the amino acid sequence of SEQ ID NO:21 and a second constant domain having the amino acid sequence of SEQ ID NO:22.

21. The recombinant protein of embodiment 12, which comprises two different constant domains, each having an amino acid sequence that has at least 97% sequence identity to any one of SEQ ID NO:3, SEQ ID NO:15, SEQ ID NO:16, SEQ ID NO:17, or SEQ ID NO:18, provided that the Fc domain (i) has the sequence P-V-A-absent at amino acids 233 to 236 as defined by EU numbering and (ii) optionally has (1) a star mutation and/or (2) a knob or hole mutation and/or (3) a S354C or E357C mutation (as defined by EU numbering).

22. The recombinant protein of embodiment 12, which comprises two different constant domains, each having an amino acid sequence that has at least 98% sequence identity to any one of SEQ ID NO:3, SEQ ID NO:15, SEQ ID NO:16, SEQ ID NO:17, or SEQ ID NO:18, provided that the Fc domain (i) has the sequence P-V-A-absent at amino acids 233 to 236 as defined by EU numbering and (ii) optionally has (1) a star mutation and/or (2) a knob or hole mutation and/or (3) a S354C or E357C mutation (as defined by EU numbering).

23. The recombinant protein of embodiment 12, which comprises two different constant domains, each having an amino acid sequence that has at least 99% sequence identity to any one of SEQ ID NO:3, SEQ ID NO:15, SEQ ID NO:16, SEQ ID NO:17, or SEQ ID NO:18, provided that the Fc domain (i) has the sequence P-V-A-absent at amino acids 233 to 236 as defined by EU numbering and (ii) optionally has (1) a star mutation and/or (2) a knob or hole mutation and/or (3) a S354C or E357C mutation (as defined by EU numbering).

24. The recombinant protein of embodiment 12, which comprises two different constant domains, each having an amino acid sequence that has 100% sequence identity to any one of SEQ ID NO:3, SEQ ID NO:15, SEQ ID NO:16, SEQ ID NO:17, or SEQ ID NO:18, provided that the Fc domain (i) has the sequence P-V-A-absent at amino acids 233 to 236 as defined by EU numbering and (ii) optionally has (1) a star mutation and/or (2) a knob or hole mutation and/or (3) a S354C or E357C mutation (as defined by EU numbering).

25. A nucleic acid, or a plurality of nucleic acids, encoding the recombinant protein of any one of embodiments 1 to 24.

26. A host cell:
(a) engineered to express the recombinant protein of any one of embodiments 1 to 24; and/or
(b) comprising one or more expression vectors encoding the recombinant protein of any one of embodiments 1 to 24.

27. The host cell of embodiment 26 which is a CHO cell or an HEK293 or derivative (e.g., Expi293F) cell.

28. A method of producing the recombinant protein of any one of embodiments 1 to 24, comprising culturing the host cell of embodiment 26 or embodiment 27 such the recombinant protein is expressed and optionally recovering and/or purifying the expressed protein.

29. A method for increasing production of a polypeptide, comprising expressing the polypeptide as a protein a defined in any one of embodiments 1 to 24.

30. The method of embodiment 29, which comprises culturing the host cell of any one of embodiments 25 to 27 such the protein is expressed and optionally recovering and/or purifying the expressed protein.

31. The method of embodiment 29 or embodiment 30, wherein the method increases expression relative to expression of a variant protein comprising a control constant domain or a pair of constant domains.

32. The method of embodiment 31, wherein the control constant domain has, or pair of constant domains have, the sequence E-L-L-G (SEQ ID NO: 23) at amino acids 233 to 236 as defined by EU numbering.

33. The method of embodiment 31, wherein the control constant domain has, or pair of constant domains have, the amino acid sequence of SEQ ID NO:1 with or without one or more mutations permitting for heterodimerization or purification, e.g., knob-in-hole mutations and/or star mutations and/or mutations that introduce a disulfide bridge (e.g., S354C or E357C).

34. The method of embodiment 31, wherein the control constant domain has, or pair of constant domains have, the amino acid sequence of SEQ ID NO:9 with or without one or more mutations permitting for heterodimerization or purification, e.g., knob-in-hole mutations and/or star mutations and/or mutations that introduce a disulfide bridge (e.g., S354C or E357C).

35. The method of embodiment 31, wherein the control constant domain has, or pair of constant domains have, the amino acid sequence of SEQ ID NO:10 with or without one or more mutations permitting for heterodimerization or purification, e.g., knob-in-hole mutations and/or star mutations and/or mutations that introduce a disulfide bridge (e.g., S354C or E357C).

36. The method of any one of embodiments 31 to 35, wherein the increase in expression and/or activity relative to the variant protein is at least 10%.

37. The method of any one of embodiments 31 to 35, wherein the increase in expression and/or activity relative to the variant protein is at least 15%.

38. The method of any one of embodiments 31 to 35, wherein the increase in expression and/or activity relative to the variant protein is at least 20%.

39. The method of any one of embodiments 31 to 35, wherein the increase in expression and/or activity relative to the variant protein is at least 30%.

40. The method of any one of embodiments 31 to 35, wherein the increase in expression and/or activity relative to the variant protein is at least 40%.

41. The method of any one of embodiments 31 to 35, wherein the increase in expression and/or activity relative to the variant protein is at least 50%.

42. A population of proteins according to any one of embodiments 1 to 24.

43. The population of proteins of embodiment 42 which is produced by the method of any one of embodiments 28 to 41.

44. The population of embodiment 42, comprising at least 10,000 proteins.

45. The population of any one of embodiments 42 to 44, which is characterized by greater activity as compared to a population of proteins comprising a control constant domain or pair of constant domains as defined in any one of embodiments 32 to 35.

46. The population of any one of embodiments 42 to 45, which is in the form of a pharmaceutical composition comprising the population of proteins and one or more excipients and/or pharmaceutically acceptable carriers.

### 8. EXAMPLES

### 8.1. Materials and Methods

### 8.1.1. Experimental Constructs

Antibody constructs comprising the IgG1 PVA domains and control constant domains as set out below were generated. Components of the test and control constructs are set forth in Table 2 below.

TABLE 2
Sequences of components of test and control constructs

| | | SEQ ID |
|---|---|---|
| Description | Amino Acid Sequence | NO. |
| hIgG1 (216-447; EU | EPKSCDKTHTCPPCPAPELLGGPSVFLFPPKPKDTLMISRTP | 1 |
| numbering) | EVTCVVVDVSHEDPEVKFNWYVDGVEVHNAKTKPREEQYNST | |
| hIgG1 hinge and Fc. | YRVVSVLTVLHQDWLNGKEYKCKVSNKALPAPIEKTISKAKG | |
| | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 PVA: | EPKSCDKTHTCPPCPAPPVAGPSVFLFPPKPKDTLMISRTPE | 2 |
| Variant hIgG1 hinge | VTCVVVDVSHEDPEVKFNWYVDGVEVHNAKTKPREEQYNSTY | |
| and Fc, with IgG2- | RVVSVLTVLHQDWLNGKEYKCKVSNKALPAPIEKTISKAKGQ | |
| based hinge region and | PREPQVYTLPPSRDELTKNQVSLTCLVKGFYPSDIAVEWESN | |
| IgG1 CH2 and CH3 | GQPENNYKTTPPVLDSDGSFFLYSKLTVDKSRWQQGNVFSCS | |
| | VMHEALHNHYTQKSLSLSPGK | |
| hIgG1 PVA star: | EPKSCDKTHTCPPCPAPPVAGPSVFLFPPKPKDTLMISRTPE | 3 |
| Variant hIgG1 hinge | VTCVVVDVSHEDPEVKFNWYVDGVEVHNAKTKPREEQYNSTY | |
| and Fc, with IgG2- | RVVSVLTVLHQDWLNGKEYKCKVSNKALPAPIEKTISKAKGQ | |
| based hinge region and | PREPQVYTLPPSRDELTKNQVSLTCLVKGFYPSDIAVEWESN | |
| IgG1 CH2 and CH3 | GQPENNYKTTPPVLDSDGSFFLYSKLTVDKSRWQQGNVFSCS | |
| with star mutation | VMHEALHNRFTQKSLSLSPGK | |
| hIgG1s: | DKKVEPKSCDKTHTCPPCPAPPVAGPSVFLFPPKPKDTLMIS | 4 |
| Variant hIgG1 hinge | RTPEVTCVVVDVSQEDPEVQFNWYVDGVEVHNAKTKPREEQF | |
| and Fc, with IgG2- | NSTYRVVSVLIVLHQDWLNGKEYKCKVSNKGLPSSIEKTISK | |
| based hinge region and | AKGQPREPQVYTLPPSRDELTKNQVSLTCLVKGFYPSDIAVE | |
| IgG4 CH2 and IgG1 | WESNGQPENNYKTTPPVLDSDGSFFLYSKLTVDKSRWQQGNV | |
| CH3 | FSCSVMHEALHNHYTQKSLSLSPGK | |
| hIgG1 N180G, also | EPKSCDKTHTCPPCPAPELLGGPSVFLFPPKPKDTLMISRTP | 5 |
| referred to as N297G: | EVTCVVVDVSHEDPEVKFNWYVDGVEVHNAKTKPREEQYGST | |
| Variant hIgG1 hinge | YRVVSVLTVLHQDWLNGKEYKCKVSNKALPAPIEKTISKAKG | |
| and Fc, with N180G | QPREPQVYTLPPSRDELTKNQVSLTCLVKGFYPSDIAVEWES | |
| mutation in depicted | NGQPENNYKTTPPVLDSDGSFFLYSKLTVDKSRWQQGNVFSC | |
| sequence (N297G by | SVMHEALHNHYTQKSLSLSPGK | |
| EU numbering) | | |
| hIgG2 (216-447; EU | ERKCCVECPPCPAPPVAGPSVFLFPPKPKDTLMISRTPEVTCVVVD | 6 |
| numbering) | VSHEDPEVQFNWYVDGVEVHNAKTKPREEQFNSTFRVVSVLTVVHQ | |
| hIgG2 hinge and Fc | DWLNGKEYKCKVSNKGLPAPIEKTISKTKGQPREPQVYTLPPSREE | |
| according to UniProt | MTKNQVSLTCLVKGFYPSDISVEWESNGQPENNYKTTPPMLDSDGS | |
| P01859 | FFLYSKLTVDKSRWQQGNVFSCSVMHEALHNHYTQKSLSLSPGK | |
| hIgG2 (216-447; EU | ERKCCVECPPCPAPPVAGPSVFLFPPKPKDTLMISRTPEVTCVVVD | 7 |
| numbering) | VSHEDPEVQFNWYVDGVEVHNAKTKPREEQFNSTFRVVSVLTVVHQ | |
| Variant hIgG2 | DWLNGKEYKCKVSNKGLPAPIEKTISKTKGQPREPQVYTLPPSREE | |
| sequence with S to A | MTKNQVSLTCLVKGFYPSDIAVEWESNGQPENNYKTTPPMLDSDGS | |
| substitution at position | FFLYSKLTVDKSRWQQGNVFSCSVMHEALHNHYTQKSLSLSPGK | |
| 159 of depicted | | |
| sequence (DISVE | | |
| (SEQ ID NO: 45) to | | |
| DIAVE (SEQ ID NO: | | |
| 46)). Unless indicated | | |
| otherwise, the variant | | |
| hIgG2 sequence was | | |
| used in the constructs | | |
| exemplified herein. | | |
| hIgG4 (216-447; EU | ESKYGPPCPSCPAPEFLGGPSVFLFPPKPKDTLMISRTPEVTCVVV | 8 |
| numbering): | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| hIgG4 S108P: | ESKYGPPCPPCPAPEFLGGPSVFLFPPKPKDTLMISRTPEVTCVVV | 9 |
| Variant hIgG4 hinge | DVSQEDPEVQFNWYVDGVEVHNAKTKPREEQFNSTYRVVSVLTVLH | |
| and Fc, with S108P | QDWLNGKEYKCKVSNKGLPSSIEKTISKAKGQPREPQVYTLPPSQE | |
| mutation (S228P by EU | EMTKNQVSLTCLVKGFYPSDIAVEWESNGQPENNYKTTPPVLDSDG | |
| numbering). | SFFLYSRLTVDKSRWQEGNVFSCSVMHEALHNHYTQKSLSLSLGK | |
| hIgG4s: | ESKYGPPCPPCPAPPVAGPSVFLFPPKPKDTLMISRTPEVTCVVVD | 10 |
| Variant hIgG4 hinge | VSQEDPEVQFNWYVDGVEVHNAKTKPREEQFNSTYRVVSVLTVLHQ | |
| and Fc, with IgG2- | DWLNGKEYKCKVSNKGLPSSIEKTISKAKGQPREPQVYTLPPSQEE | |
| based hinge region with | MTKNQVSLTCLVKGFYPSDIAVEWESNGQPENNYKTTPPVLDSDGS | |
| S108P mutation | FFLYSRLTVDKSRWQEGNVFSCSVMHEALHNHYTQKSLSLSLGK | |
| (S228P by EU | | |
| numbering), and IgG1 | | |
| CH2 and CH3 | | |
| G₄S (SEQ ID NO: 11): | GGGGS | 11 |
| 7-amino acid linker | GGGGSGG | 12 |
| 15-amino acid linker | 3 × G₄S (i.e., GGGGSGGGGSGGGGS) | 13 |
| 30-amino acid linker | 6 × G₄S (i.e., | 14 |
| | GGGGSGGGGSGGGGSGGGGSGGGGSGGGGS) | |
| IgG1PVA_hinge-Fc | EPKSCDKTHTCPPCPAPPVAGPSVFLFPPKPKDTLMISRTPE | 15 |
| knob | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNHYTQKSLSLSPGK | |
| with knob mutation | | |
| T366W | | |
| IgG1PVA_hinge-Fc | EPKSCDKTHTCPPCPAPPVAGPSVFLFPPKPKDTLMISRTPE | 17 |
| knob_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNRFTQKSLSLSPGK | |
| with knob mutation | | |
| T366W, and star | | |
| mutations H435R, | | |
| Y436F | | |
| IgG1PVA_hinge-Fc | EPKSCDKTHTCPPCPAPPVAGPSVFLFPPKPKDTLMISRTPE | 19 |
| knob_Cys | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPCRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNHYTQKSLSLSPGK | |
| with knob mutation | | |
| T366W, and Cys | | |
| mutation S354C | | |
| IgG1PVA_hinge-Fc | EPKSCDKTHTCPPCPAPPVAGPSVFLFPPKPKDTLMISRTPE | 21 |
| knob_Cys_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPCRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNRFTQKSLSLSPGK | |
| with knob mutation | | |
| T366W, Cys mutation | | |
| S354C mutation and | | |
| star mutations H435R, | | |
| Y436F | | |
| IgG1PVA_hinge-Fc | EPKSCDKTHTCPPCPAPPVAGPSVFLFPPKPKDTLMISRTPE | 16 |
| Hole | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNHYTQKSLSLSPGK | |
| with hole mutations | | |
| T366S, L368A, Y407V, | | |
| IgG1PVA_hinge-Fc | EPKSCDKTHTCPPCPAPPVAGPSVFLFPPKPKDTLMISRTPE | 18 |
| Hole_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNRFTQKSLSLSPGK | |
| with hole mutations | | |
| T366S, L368A, Y407V, | | |
| and star mutations | | |
| H435R, Y436F | | |
| IgG1PVA_hinge-Fc | EPKSCDKTHTCPPCPAPPVAGPSVFLFPPKPKDTLMISRTPE | 20 |
| Hole_Cys | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVCTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNHYTQKSLSLSPGK | |
| with hole mutations | | |
| T366S, L368A, Y407V, | | |
| and Cys mutation | | |
| S354C | | |
| IgG1PVA_hinge-Fc | EPKSCDKTHTCPPCPAPPVAGPSVFLFPPKPKDTLMISRTPE | 22 |
| Hole_Cys_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVCTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNRFTQKSLSLSPGK | |
| with hole mutations | | |
| T366S, L368A, Y407V, | | |
| Cys mutation S354C | | |
| and star mutations | | |
| H435R, Y436F | | |

The test and control constructs included various bispecific and trispecific binding molecules, as set out below in Table 3, which provides descriptions of the various controls and test constructs utilized throughout the studies described herein. Certain bispecific antibodies included three antigen-binding sites, the first of which binds to FGFR1c, the second of which binds to the GH1 domain of KLB, and the third of which binds to the GH2 domain of KLB; an IgG Fc domain (e.g., IgG1 PVA, IgG1 N180G (N297G by EU numbering), IgG4, or IgG4s); and linkers of different lengths. In some instances, the antigen-binding site which binds to the GH2 domain of KLB is a Fab or an scFv, and is connected to the N-terminus of a Fab which bind to FGFR1c on a first arm of the bispecific antibody, while a Fab which binds to the GH1 domain of KLB is located on a second arm of the bispecific antibody.

TABLE 3
Description of control and test antibody constructs

| Molecule | Description |
|---|---|
| hIgG1 control | IgG1 hinge and Fc (hIgG1 216-447; EU numbering). |
| hIgG4 control | IgG4 S108P hinge and Fc. |
| His hFGF21 | Human FGF21 with 6xHis Tag (SEQ ID NO: 47). |
| AF1d, IgG1 | Alternative format antibody |
| | Includes hIgG1 hinge and Fc. |
| | Antibody has a 2 + 1 N-scFv format. |
| | scFv is connected to an inner Fab via the |
| | 30-amino acid linker, |
| | while the other Fab is on the opposite arm of the antibody |
| AF2d, IgG1 | Alternative format antibody. |
| | Includes hIgG1 hinge and Fc. |
| | Antibody has a 2 + 1 N-Fab format. |
| | Outer Fab is connected to an inner Fab via the |
| | 30-amino acid linker, while the third Fab is on the |
| | opposite arm of the antibody. |
| AF1-7, IgG1 | Alternative format antibody |
| PVA | Includes hIgG1 PVA hinge and Fc. |
| | Antibody has a 2 + 1 N-scFv format. |
| | scFv is connected to an inner Fab via the |
| | 7-amino acid linker, while the other |
| | Fab is on the opposite arm of the antibody. |
| AF1-15, IgG1 | Alternative format antibody |
| PVA | Includes hIgG1 PVA hinge and Fc. |
| | Antibody has a 2 + 1 N-scFv format. |
| | scFv is connected to an inner Fab |
| | via the 15-amino acid linker, while the |
| | other Fab is on the opposite arm of the antibody. |
| AF1-30, IgG1 | Alternative format antibody |
| PVA or AF1a, | Includes hIgG1 PVA hinge and Fc. |
| IgG1 PVA | Antibody has a 2 + 1 N-scFv format. |
| | scFv is connected to an inner Fab |
| | via the 30-amino acid linker, while the |
| | other Fab is on the opposite arm of the antibody. |
| AF1-7, IgG4 | Alternative format antibody |
| S108P | Includes hIgG4 S108P hinge and Fc. |
| | Antibody has a 2 + 1 N-scFv format. |
| | scFv is connected to an inner Fab via the 7-amino acid linker, |
| | while the other Fab is on the opposite arm of the antibody. |
| AF1-15, IgG4 | Alternative format antibody |
| S108P | Includes higG4 S108P hinge and Fc. |
| | Antibody has a 2 + 1 N-scFv format. |
| | scFv is connected to an inner Fab via the 15-amino acid linker, |
| | while the other Fab is on the opposite arm of the antibody |
| AF1-30, IgG4 | Alternative format antibody |
| S108P or | Includes hIgG4 S108P hinge and Fc. |
| AF1-b, | Antibody has a 2 + 1 N-scFv format. |
| IgG4 S108P | scFv is connected to an inner Fab via the 30-amino acid linker, |
| | while the other Fab is on the opposite arm of the antibody. |
| AF2a, IgG1 | Alternative format antibody. |
| PVA | Includes hIgG1 PVA hinge and Fc. |
| | Antibody has a 2 + 1 N-Fab format. |
| | Outer Fab is connected to an inner Fab via the 30-amino acid |
| | linker, while the third Fab is on the opposite arm of the |
| | antibody. |
| AF2b, IgG4 | Alternative format antibody. |
| S108P | Includes hIgG4 S108P hinge and Fc. |
| | Antibody has a 2 + 1 N-Fab format. |
| | Outer Fab is connected to an inner Fab via the 30-amino acid |
| | linker, while the third Fab is on the opposite arm of the |
| | antibody |
| AF1c, IgG1 | Alternative format antibody |
| N180G | Includes hIgG1 hinge and Fc N180G. |
| | Antibody has a 2 + 1 N-scFv format. |
| | scFv is connected to an inner Fab via the 15-amino acid linker, |
| | while the other Fab is on the opposite arm of the antibody. |
| AF2c, IgG1 | Alternative format antibody. |
| N180G | Includes hIgG1 hinge and Fc N180G. |
| | Antibody has a 2 + 1 N-Fab format. |
| | Outer Fab is connected to an inner Fab via the 30-amino acid |
| | linker, while the third Fab is on the opposite arm of the |
| | antibody |
| AF3a, IgG1 | Alternative format antibody. |
| PVA | Includes hIgG1 PVA hinge and Fc. |
| | Antibody has a 2 + 1 N-Fab format. |
| | Outer Fab is connected to an inner Fab via the 15-amino acid |
| | linker, while the third Fab is on the opposite arm of the |
| | antibody. |
| AF3b, IgG4 | Alternative format antibody. |
| S108P | Includes hIgG4 S108P hinge and Fc. |
| | Antibody has a 2 + 1 N-Fab format. |
| | Outer Fab is connected to an inner Fab via the 15-amino acid |
| | linker, while the third Fab is on the opposite arm of the |
| | antibody |
| AF4, IgG4s | Alternative format antibody. |
| | Includes IgG4s hinge and Fc. |
| AF5-a, IgG1 | Alternative format antibody. |
| PVA | Includes hIgG1 PVA hinge and Fc. |
| AF5-b, IgG4 | Alternative format antibody. |
| S108P | Includes hIgG4 S108P hinge and Fc. |
| mAb1, IgG1 | IgG1 control. |
| mAb2, IgG4 | IgG4 S108P control. |
| S108P | |
| mAb3, IgG4s | IgG4s control. |

### 8.1.2. Vector Construction

DNA fragments encoding anti-KLB GH1 Fab, anti-KLB GH2 Fab, anti-KLB GH2 scFv, and anti-FGFR1c Fab domains; various amino acid linkers; and various IgG hinge and Fc domains were synthesized by Integrated DNA Technologies, Inc. (San Diego, California) or Geneart/Thermo Fisher Scientific (Regensburg, Germany)

Mammalian expression vectors for individual polypeptide chains were created by one of the following approaches: using NEBuilder HiFi DNA Assembly Kit (New England BioLabs Inc.); by restriction digest followed by ligation following standard molecular cloning protocols provided by New England BioLabs Inc; or by DNA synthesis and cloning in ready-to-use constructs into pcDNA3.4 Topo expression system (Life Technologies). DNAs were transfected as a single plasmid or as a heavy and light chain pair, following the manufacturer's protocol. 50 ml of cell culture supernatant was harvested and processed for purification via HiTrap™ Protein G HP, HiTrap Protein A FF, or MabSelect SuRe pcc columns (Cytiva).

Certain constructs were expressed in Expi293F™ cells by transient transfection (Thermo Fisher Scientific). Proteins in Expi293F supernatant were purified using the ProteinMaker system (Protein BioSolutions, Gaithersburg, MD) with either HiTrap™ Protein G HP or MabSelect SuRe pcc columns (Cytiva). After single step elution, antibodies were neutralized, dialyzed into a final buffer of phosphate buffered saline (PBS) with 5% glycerol, aliquoted and stored at −80° C. For some constructs, an additional step of size-exclusion chromatography with HiPrep 26/60 Sephacryl S-200 column was used.

Other expression vectors were stably expressed in a Chinese hamster ovary (CHO) expression system. Purification of these antibodies from CHO stable expression followed similar procedures supra.

### 8.1.3, Kinetics Measurement of Fc Receptor Binding by Biacore

Briefly, surface plasmon resonance (SPR) experiments were performed at 25° C. on a Biacore T200 instrument employing a carboxymethyl dextran-coated (CM-5) chip. A mouse monoclonal anti-penta-histidine antibody (GE Healthcare) was immobilized on the surface of the CM-5 sensor chip using standard amine-coupling chemistry. 140RU-376RU of His-tagged human, monkey or mouse FcγR proteins were captured on the anti-penta-histidine amine-coupled CM-5 chip and stock solutions of antibodies were injected at 50 μl/min for 2 min over the captured proteins and serially diluted (6 uM-24.7 nM). mAb binding response was monitored and, for low affinity receptors, steady-state binding equilibrium was calculated. Kinetic association (ka) and dissociation (kd) rate constants were determined by processing and fitting the data to a 1:1 binding model using Scrubber 2.0 curve fitting software. Binding dissociation equilibrium constants (KD) and dissociative half-lives (t1/2) were calculated from the kinetic rate constants as: KD (M)=kd/ka; and t1/2 (min)=(ln2/(60*kd). Some KDs were derived using the steady state equilibrium dissociation constant: NB=no binding observed; IC=inconclusive affinity determination due to low specific RU signal.

### 8.1.4. Enzyme-Linked Immunosorbent Assay (ELISA)

Wells of microtiter plates were coated (18 h, 4° C.) with 4 μg/ml of 6×-His Tag (SEQ ID NO: 47) monoclonal antibody (4E3D10H2/E3) (Thermo scientific) in 100 μl of PBS and were then blocked with blocking buffer (2% BSA in PBS) for 1 h at room temperature. Different Fc receptors (2 μg/ml, 100 μl/well) were loaded in duplicates and incubated for 1 h at room temperature. Meanwhile, the antibodies were diluted with a ratio of 1:5 from a starting concentration of 6.0×10⁻⁰⁶ M in blocking buffer. The diluted antibodies (100 ul) were then added into the wells and incubated for 1 h at room temperature. Peroxidase-conjugated Goat Anti-Human IgG, F(ab′)₂ detection antibody 100 ul/well (1:5000 in blocking buffer) was added for 1 h at room temperature and the reaction was visualized by the addition of 100 μl peroxidase substrate (KPL-TMB) for 30 min. The reaction was stopped with 100 μl TMB stop buffer and measured the absorbance at 450 nm using ELISA plate reader (Envision, PerkinElmer). Plates were washed three times with wash buffer (PBS, pH 7.4, containing 0.05% (v/v) Tween 20) after each step.

### 8.1.5. Surrogate ADCC Assay

### 8.1.5.1. Target Cells

HEK293/hFGFR1c/hKLB/hCD20: HEK293 cells where endogenous FGFR1 was excised by CRISPR-Cas9, were engineered to constitutively express full length human CD20 (hCD20, amino acids M1-P297 of accession number NP_690605.1), FGFR1c (hFGFR1c, amino acids M1-R731 of accession number NP_075594), and KLB (hKLB, amino acids M1-S1044 of accession number NP_783864.1) Cells were sorted for high expression of all receptors.

### 8.1.5.2. Reporter Cells

Jurkat/NFAT-Luc/FcγR3a 176Val: Jurkat T cells were engineered to stably express a Nuclear Factor of Activated T-cells (NFAT) luciferase reporter construct along with the high affinity human FcγR3a 176Val allotype receptor (amino acids M1-K254 of accession number P08637 VAR_003960).

### 8.1.5.3. Assay Set-Up

Three days before the experiment, Jurkat reporter cells were split to 1.25×10⁵ cells/ml in RPM11640+10% FBS+P/S/G+0.5 μg/ml puromycin+500 μg/ml G418 growth media. On the day of the experiment, the target and reporter cells were transferred into assay media (RPMI +10% FBS+P/S/G) and added at a 1:1 ratio (3×10⁴/well of each cell type) to 96-well white microtiter plates. Multi-specific anti-FGFR1c/KLB antibodies and an hIgG4 S108P isotype control antibody were titrated in a 7-point, 1:4 serial dilution ranging from 73.2 pM to 300 nM final concentration, with the final 8th point containing no antibody, and added to the cells in duplicate. Plates were incubated at 37° C./5% C02 for 4.6 h followed by the addition of an equal volume of ONE-Glor™ (Promega) reagent to lyse cells and detect luciferase activity. The emitted light was captured in Relative Light Units (RLU) on a multi-label plate reader Envision (PerkinElmer). EC50 values of the antibodies were determined from a 4 parameter logistic equation over an 8-point dose response curve (including the background signal) using GraphPad Prism software. Maximum fold induction was calculated using the following equation:

Fold Induction=Max Average RLU within tested dose range of each antibody/Average RLU (background signal=no antibody)

### 8.1.6. Stable Expression and Antibody Titer

Recombinant proteins encoding different antibodies with various IgG subclasses were cloned into expression plasmids, transfected into CHO cells and stably transfected pools were isolated after selection with 400 mg/L hygromycin for 12-14 days. The stable CHO-cell pools, grown in chemically-defined protein-free medium in suspension, were used to produce proteins for testing.

Protein was produced by inducing cell cultures with 0.5 mg/L Doxycycline for five days and harvesting the conditioned media. Protein titers were determined with an Octet instrument (ForteBio) using a protein A sensor against a known standard at various concentrations.

### 8.1.7. Luciferase Reporter Assay

Antibodies including different IgG hinge and Fc domains were tested for their agonist activities using HEK293.SREluc.hFGFR1c/hKLB cells that stably expressed human FGFR1c and KLB as well as a luciferase reporter gene under the control of a promoter containing serum responsive elements (SRE). Recombinant human FGF21 with 6×His tag (SEQ ID NO: 47) was used as a positive control, with the maximum reporter activity obtained from FGF21 defined as 100% activity. Cells were treated with each antibody or 6×His-FGF21 (“HHHHHH” disclosed as SEQ ID NO: 47) for 6 hours, and then subjected to luciferase assays. The percent activity induced by individual antibodies was normalized against the maximum activity by FGF21. Dose-response assays were performed to determine EC50. The anti FeID1 isotype (hIgG4-S108P) control antibody, was used as a negative control.

### 8.1.8. Human Primary Adipocytes Signaling Assay

Human primary adipocytes differentiated from subcutaneous preadipocytes were obtained from Zen-Bio Inc (Durham, NC). Cells were cultured in a serum free media for 4 hours, and then treated with serially diluted antibodies for 15 minutes. Cells were lysed using a lysis buffer for AlphaScreen™ SureFire™ ERK Assay kit that measures phospho-ERK in the treated cell lysates (PerkinElmer, Shelton, CT). SureFire™ ERK Assay was performed according to the manufacturer's protocol. His-tagged human FGF21 and an isotype control human IgG4 antibody were tested as a positive and negative controls, respectively. An FGFR1c/KLB bispecific antibody was also included in the experiment.

### 8.1.9. Flow Binding Assay

BaF3 cells overexpressing target protein X were resuspended in FACS wash buffer (PBS with 1% FBS) at 1×10⁶ cells/mL. The staining was performed with 1×10⁵ cells per well. The antibodies were diluted with a ratio of 1:5 from a starting concentration of 1.3×10⁻⁰⁷ M. The diluted antibodies were then added into the wells containing cells. Cells were stained for 30 min at 2-8° C. and washed twice with FACS wash buffer. AF647-conjugated goat anti-hFc Fab (Jackson Immuno Research, 109-607-003, 1:400) was added to wells and cells were incubated for 30 min at 2-8° C. Next, the cells were washed to remove excess antibodies and fixed in 2% paraformaldehyde for 30 min at 2-8° C. After two washes, stained cells were analyzed using BD LSR Fortessa™ FACS instrument. The results were analyzed by FlowJo. FSC/SSC gates were used to select mononuclear cells.

### 8.1.10. Negative Stain Electron Microscopy (EM)

Complexes of C-terminally myc-myc-6×His-tagged CD40 ectodomain (res. 20-193) with anti-CD40 antibodies containing different hinge sequences (IgG1, IgG1-PVA, and IgG2) were isolated by size exclusion chromatography using a Superdex 200 increase 10/300 column. Purified samples at a protein concentration of approximately 0.02 mg/mL were applied to 400 mesh carbon film Cu grids (Electron Microscopy Sciences) and negative-stained with NanoW (Nanoprobes) or Vitroease Methylamine Tungstate (Thermo Fisher).

Negative stain EM grids were inserted into a Glacios TEM (Thermo Fisher) and imaged with a Ceta camera (Thermo Fisher). Automated data collection was performed at a nominal magnification of 73,000× using EPU. EM data were processed using RELION 4.0. Particles were first picked using the Laplacian of Gaussian algorithm to generate 20 templates that were subsequently used for template-based particle picking. Particle images were subjected to multiple rounds of 20 classification, selecting particles belonging to class averages with clear features of IgG after each round. Diffuse density for bound CD40 allowed for assignment of the two Fab arms, which otherwise lack distinguishing features from the Fc region. Class averages with discernible features of two Fabs each bound to CD40 were selected for Fab-Fab angle determination. Fab-Fab angle, measured manually using the Angle tool in ImageJ, is the angle between lines drawn through the long axis of the two CD40-bound Fab arms.

### 8.2. Example 1: Design, Cloning, and Expression of IgG1 PVA 8.2.1. Overview

IgG1 Fc and IgG4 Fc have differing Fc gamma receptor binding capacity and charge distribution, which provide options for optimal Fc function engagement and varied compatibility with antibody building blocks such as Fabs, scFvs, and alternative format antibody fusion proteins. The hinge regions of IgG1 and IgG4 also have differing lengths and flexibility. IgG4 (S108P, or S228P, EU numbering) has been utilized in multiple approved antibody products, such as pembrolizumab, nivolumab and Ixekizumab, where reduced Fc effector function is needed. Due to the preference of antibody building blocks (e.g., Fabs, scFvs) for particular immunoglobulin subclasses, human IgG1 Fc-based alternative and natural sequence variant differing from IgG4 (S108P)—which demonstrates reduced Fc gamma receptor binding and reduced Fc receptor effector functions—was sought.

FIG. 4 presents an alignment of various IgG hinge/Fc variants with sequences between various wild type and modified human IgG1 and IgG4 hinge regions and a description of CH2 and CH3 Fc regions used, from amino acid 226 to 447 (EU numbering). hIgG1 PVA was designed to include the PVA mutation in the lower hinge region in an otherwise fully-IgG1 background (e.g., IgG1 upper hinge, CH2, and CH3 regions).

To test the properties of the hIgG1 PVA, it was incorporated into alternative format antibodies having either a 2+1 N-scFv or 2+1 N-Fab format (see, e.g., FIG. 5 for an illustration of the 2+1 N-scFv format; in the 2+1 N-Fab format, the N-terminal scFv domain is replaced by a Fab domain).

### 8.2.2. Results

Controls and bispecific antibodies incorporating the various Ig hinge and Fc domains were successfully expressed and purified.

When expressed in CHO cells, AF1 constructs in IgG1 PVA backbone with varied linker lengths between the scFv and the Fab had higher antibody titer (measured as the total antibody species) than constructs including IgG4 S108P (FIG. 5).

### 8.3. Example 2: Binding Kinetics for Fc Gamma Receptors

### 8.3.1. Overview

Binding affinities and signals of various antibodies with different hinge-Fc regions to Fc gamma receptors were measured by Biacore as described in Section 8.1.3.

### 8.3.1.1. Results

The results are shown in Tables 4 and 5 below.

TABLE 4
Binding signal of antibody with various Fc variants to human FcγR Binding

| | | FOR capture (RU) | | | | | | |
|---|---|---|---|---|---|---|---|---|
| | | | 164.4 ± 0.4 | 364.4 ± 0.6 | | 208 ± 1.7 | 899 ± 2.7 | |
| | | 615.9 ± 0.8 | Human | Human | 378.2 ± 1 | Human | Human | 548 ± 2.4 |
| | | Human | FcγRIIa | FcγRIIa | Human | FcγRIIIa | FcγRIIIa | Human |
| Antibody | Fc variant | FcγRI | (R131) | (H131) | FcγRIIb | (F176) | (V176) | FcγRIIIb |
| mAb1 | hlgG1 | 1606 | 457 | 614 | 833 | 322 | 2228 | 1028 |
| mAb2 | hlgG4 | 1475 | 181 | 393 | 737 | 28 | 429 | −2 |
| | S108P | | | | | | | |
| mAb3 | hlgG4s | 36 | 340 | 419 | 226 | 6 | 34 | −8 |
| AF2a | hlgG1 PVA | 6 | 91 | 21 | −1 | 4 | 144 | −5 |
| AF2c | hlgG1 | 518 | −5 | −9 | −9 | −5 | −11 | −7 |
| | N180G | | | | | | | |

TABLE 5
KD (M) Values for High and Low Affinity FcγR Binding

| | | | Human | Human | | Human | Human | |
|---|---|---|---|---|---|---|---|---|
| | | Human | FcγRIIa | FcγRIIa | Human | FcγRIIIa | FcγRIIIa | Human |
| Antibody | Fc variant | FcγRI | (R131) | (H131) | FcγRIIb | (F176) | (V176) | FcγRIIIb |
| mAb1 | hlgG1 | 1.76E−09 | 3.40E−07 | 3.40E−07 | 5.40E−07 | 1.02E−06 | 6.20E−07 | 1.02E−06 |
| mAb2 | hlgG4 | 5.35E−09 | 5.70E−06 | 1.50E−06 | 7.20E−07 | WB | 4.90E−06 | NB |
| | S108P | | | | | | | |
| mAb3 | hlgG4s | NB | 1.32E−06 | 1.61E−06 | 1.23E−05 | NB | WB | NB |
| AF2a | hlgG1 PVA | NB | WB | WB | NB | NB | 7.20E−05 | NB |
| AF2c | hlgG1 | 2.21E−06 | NB | NB | NB | NB | NB | NB |
| | N180G | | | | | | | |

In Table 5, NB refers to No Binding; WB refers to Weak Binding

IgG1 PVA has no binding signal in FcγR1, FcγR2b, FcγR3a (F176), FcγR3b. It has low binding signal to FcγR2a (both R131 and H131) but at a significantly reduced level (91 and 21 RU respectively) in comparison to IgG1 and IgG4 S108P. IgG1 PVA has weak to medium binding signal (144 RU) to FcγR3a (V176) with a KD=7.2×10⁻⁰⁵ M, much weaker than that for IgG1 and IgG4 S108P (Tables 4 and 5).

### 8.4. Example 3: ELISA Binding to Fc Gamma Receptors

### 8.4.1. Overview

Binding of FGFR1c/KLB bispecific antibodies including various IgG hinge and Fc regions was assessed by ELISA as described in Section 5.1.4.

### 8.4.2. Results

Binding curves indicating the ability of the controls and test antibodies to bind various Fc gamma receptors are depicted in FIGS. 6A-6G. Antibodies harboring the wild type IgG1 hinge and Fc domain demonstrated the highest binding to hFCRγ1. Binding of hFCRγ1 was significantly reduced with IgG1 PVA, showing similar binding to IgG4s (FIG. 6A). Binding with IgG1 N180G was similarly reduced. IgG4 S18P demonstrated only slightly reduced binding to hFCRγ1 relative to wild type IgG1. A similar trend was observed in the binding of hFCRγ3A (V158) and hFCRγ3A (F158) (FIG. 6E, 6F). Very little difference in binding was observed with hFCRγ2A (H131), hFCRγ2A (R131) (FIGS. 6B and 6C). But IgG1 PVA has weaker binding than IgG4 S108P and slightly weaker than IgG1 in hFCRγ2B (FIG. 6D). In hFCRγ3B, IgG1 PVA has less binding than IgG1 (FIG. 6G).

### 8.5. Example 4: Antibody-Dependent Cellular Cytotoxicity

### 8.5.1. Overview

Utilizing the surrogate antibody-dependent cellular cytotoxicity (ADCC) assay described in Section 8.1.5, cytotoxic activity of IgG1 PVA was determined and compared to the cytotoxic activity of other IgG variants (e.g., IgG1 N180G and IgG4 S108P).

The ability of multispecific antibodies targeting hFGFR1c and hKLB to interact with FcγR3a, an Fc-receptor prominently expressed on NK cells that induces antibody dependent cell-mediated cytotoxicity (ADCC), was measured in a surrogate bioassay using reporter cells and target cells bound to antibodies. In this assay, engineered Jurkat T cells express the reporter gene luciferase under the control of the transcription factor NFAT (NFAT-Luc) along with the high affinity human FcγR3a 176Val allotype receptor (Jurkat/NFAT-Luc/hFcyR3a ¹⁷⁶Val). Target cells are HEK293 cells engineered to express human CD20 in combination with full length human FGFR1c and human KLB. Reporter cells are incubated with target cells and engagement of FcγR3a via the Fc domain of human IgG1 antibodies bound to target cells leads to the activation of the transcription factor NFAT in the reporter cells and drives the expression of luciferase which is then measured via a luminescence readout.

### 8.5.2. Results

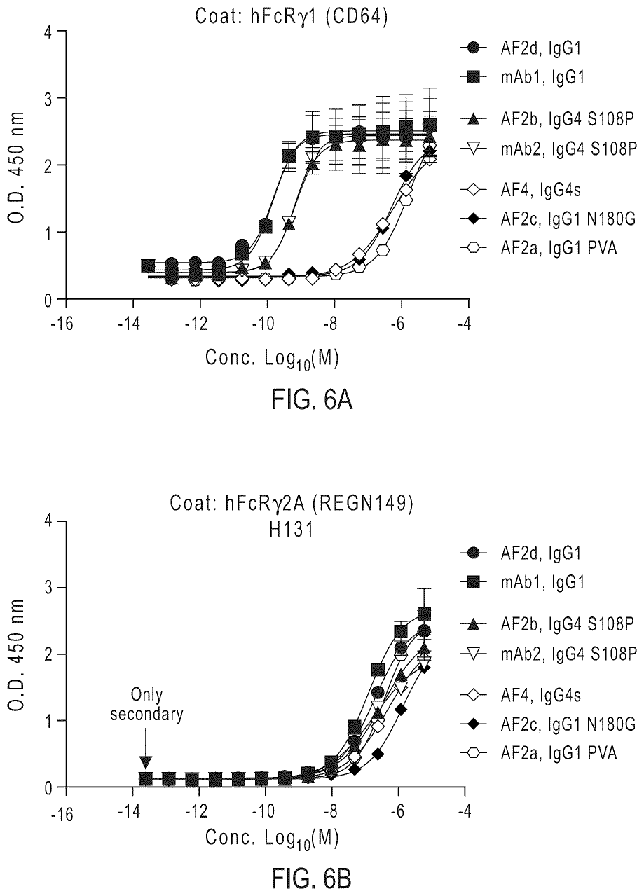

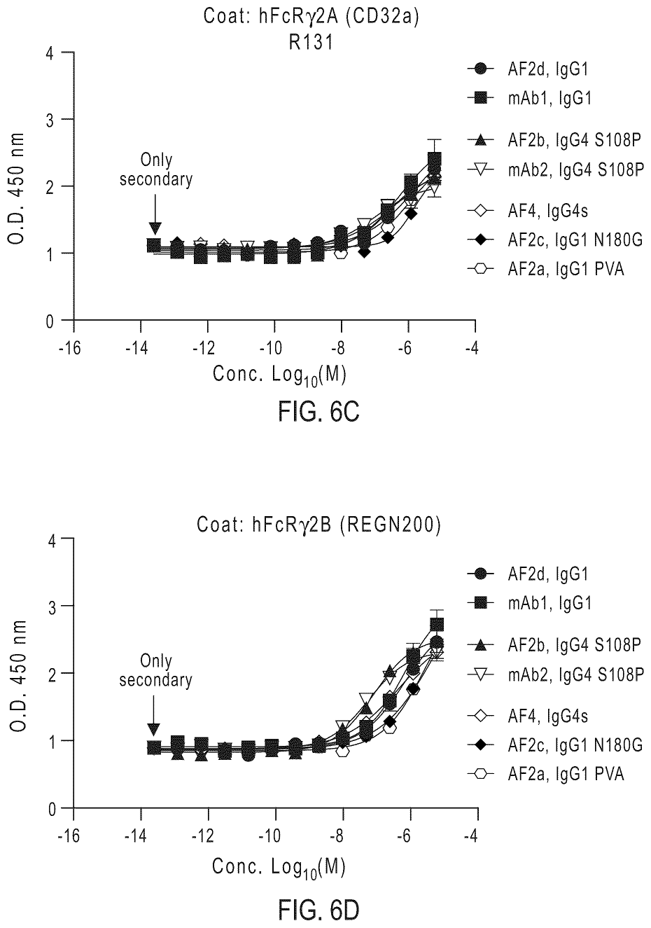

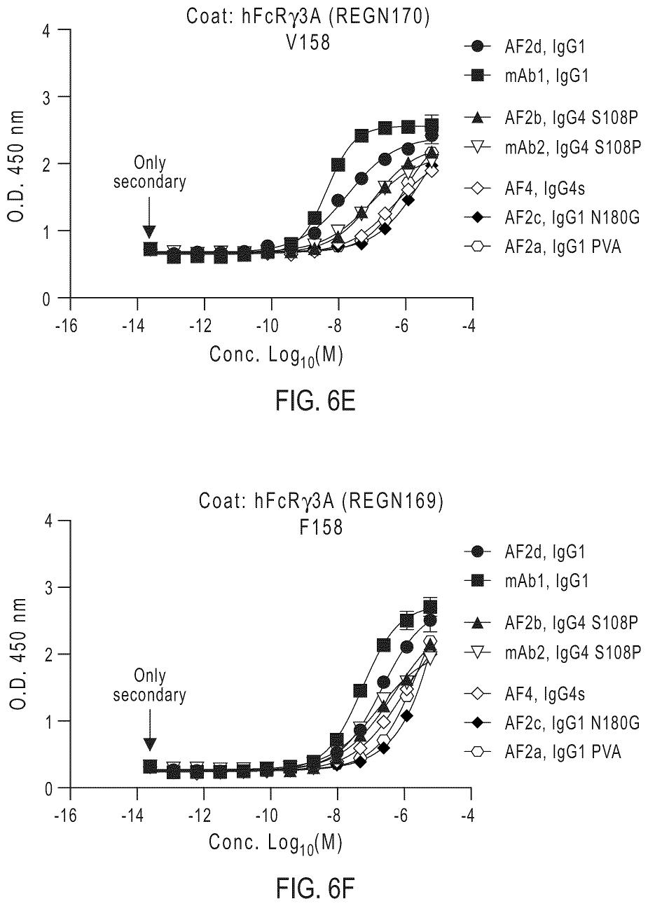

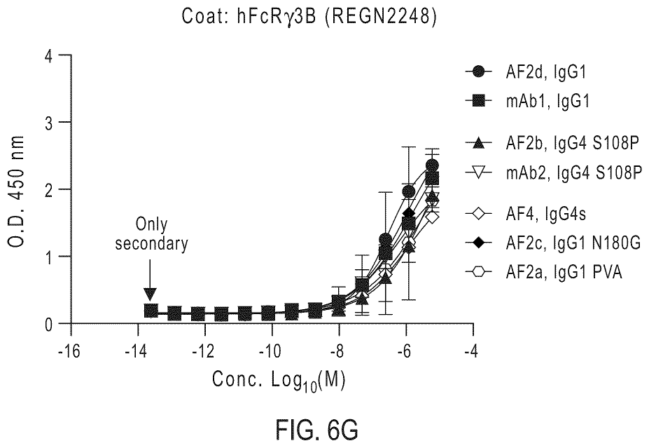

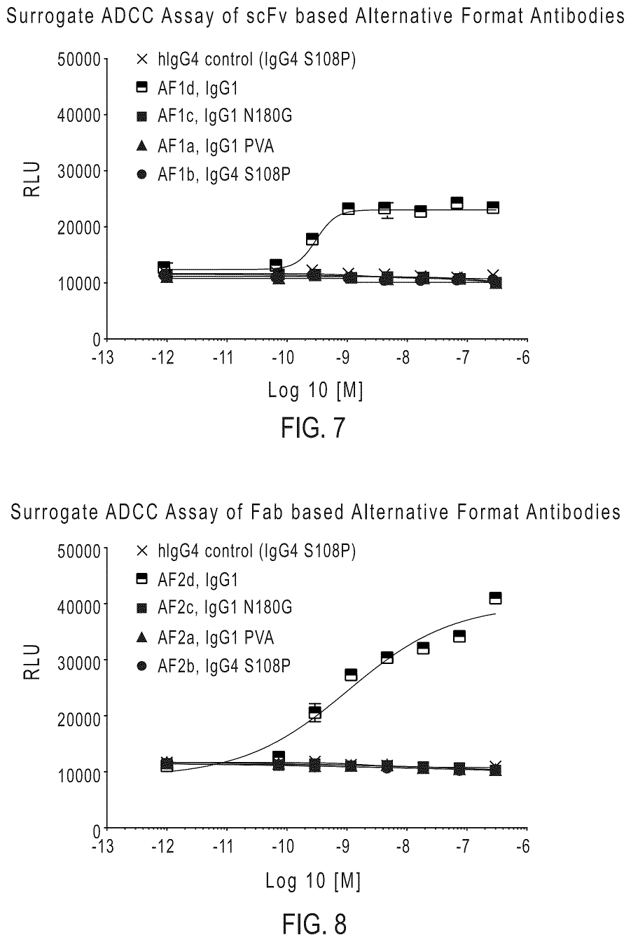

Representative data from the ADCC assays are depicted in FIGS. 7 and 8. Only alternative format antibodies with wild type IgG1, AF1d IgG1 and AF2d IgG1 showed induction of luciferase signal, by 1.9 fold (EC50=307 pM) and 3.4 fold (EC50=1.04 nM), respectively. None of the alternative format antibodies in 2+1 N-scFv or 2+1 N-Fab formats with IgG1 PVA, IgG1 N1800G or IgG4 S108P showed activity in the surrogate ADCC assay.

### 8.6. Example 5: Molecule Activity

### 8.6.1. Overview

The activity of FGFR1c/KLB bispecific antibodies including IgG1 PVA and controls was tested utilizing the luciferase reporter assay and human primary adipocyte signaling assay described in Sections 6.1.7 and 8.1.8.

### 8.6.2. Results

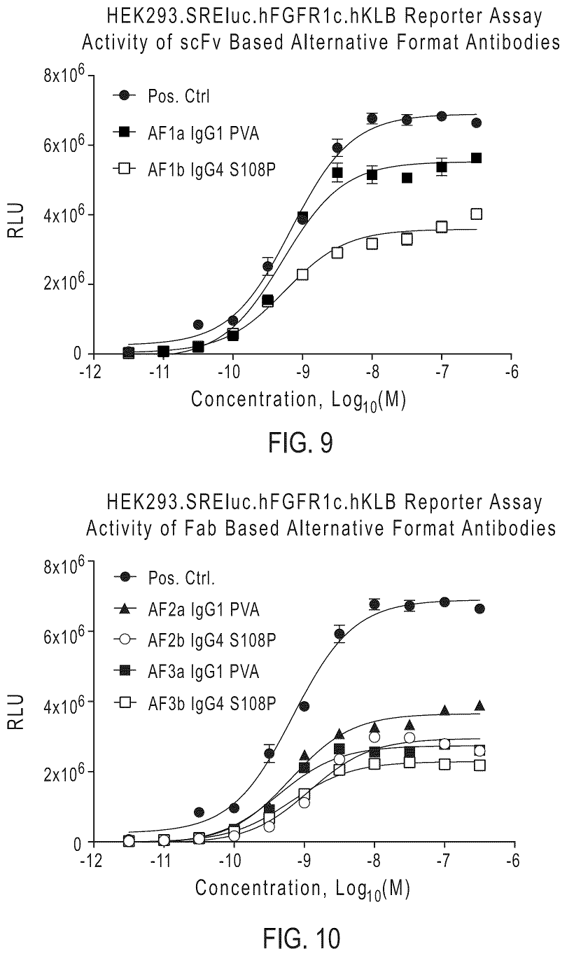

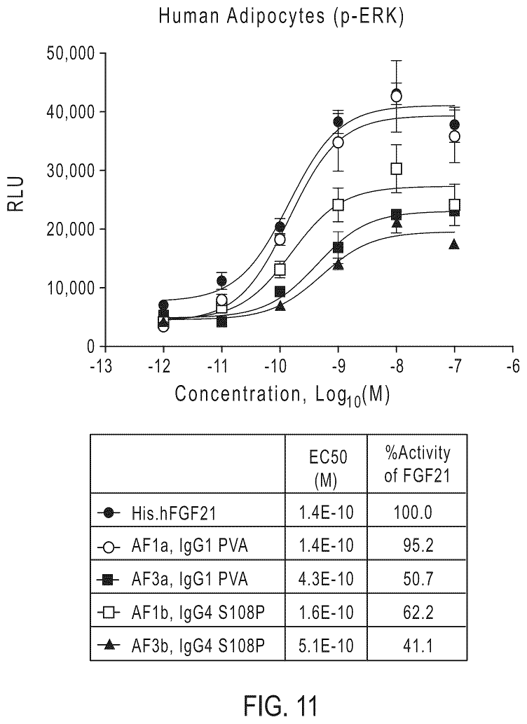

Activity of alternative format antibodies in HEK.293SREluc.hFGFR1c/hKLB is shown in FIG. 9 (2+1 N-scFv, AF1a, 1b) and FIG. 10 (2+1 N-Fab, AF2a, 2b, 3a, 3b). Activity in human adipocytes is shown in FIG. 11 (AF1a, 1b, 3a, 3b). The antibody with 2+1 N-scFv format incorporating IgG1 PVA (AF1a) showed superior agonist activity to that with IgG4 S108P (AF1b) in both HEK FGFR1c/KLB cells (FIG. 9) and human adipocytes (FIG. 11). Antibody AF2a (2+1 N-Fab format in IgG1 PVA) caused greater maximum activation than AF2b (the same antibody as an IgG4 S108P) in reporter cell assay (FIG. 10).

### 8.7. Example 6: Target Cell Binding

### 8.7.1. Overview

AF5-a and AF5-b, both multispecific, tetravalent antibodies with four antigen binding domains that bind to protein X, were designed and produced as described in Sections 8.1.1 and 8.1.2, wherein AF5-a and AF5-b were designed to comprise the same set of antigen binding arms that were linked to either human IgG1 PVA or IgG4 S10SP backbones, respectively. The binding properties of AF5-a, AF5-b, and non-binding isotype control antibodies were assessed using the flow binding assay described in Section 8.1.9.

### 8.7.2. Results

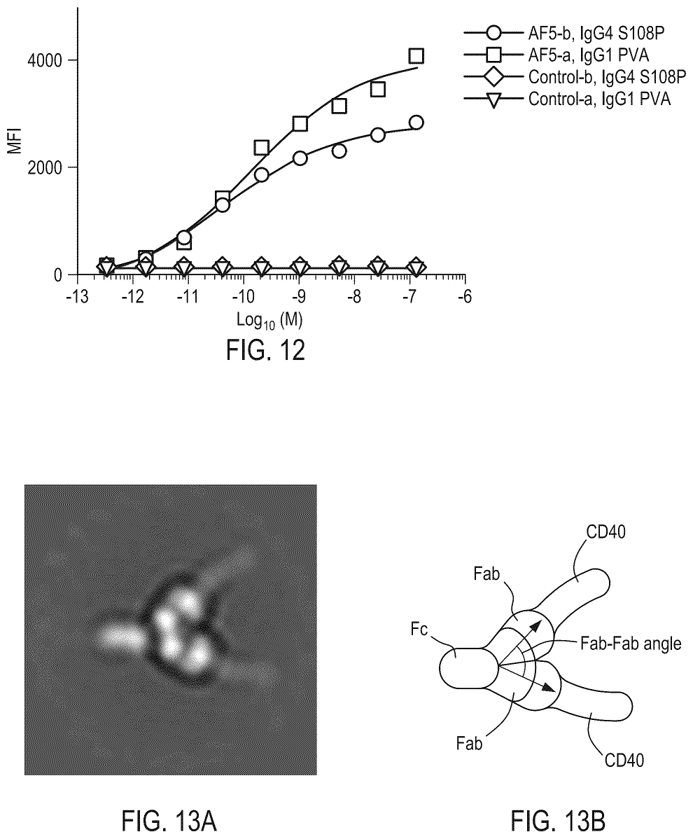

As expected, the isotype control antibodies displayed no binding affinity. In contrast, both AF5-a and AF5-b displayed binding to protein X-overexpressing cells. More specifically, AF5-a had a higher maximum MFI signal than AF5-b (FIG. 12), indicating that the IgG1 PVA backbone was associated with higher efficacy than the IgG4 S108P backbone.

### 8.8. Example 7: Fab-Fab Angles Associated with Three Different Hinge Sequences

### 8.8.1. Overview

Fab-fab angles of CD40-bound anti-CD40 IgG antibodies linked to different IgG hinge sequences were calculated as described in Section 8.1.10.

### 8.8.2. Results

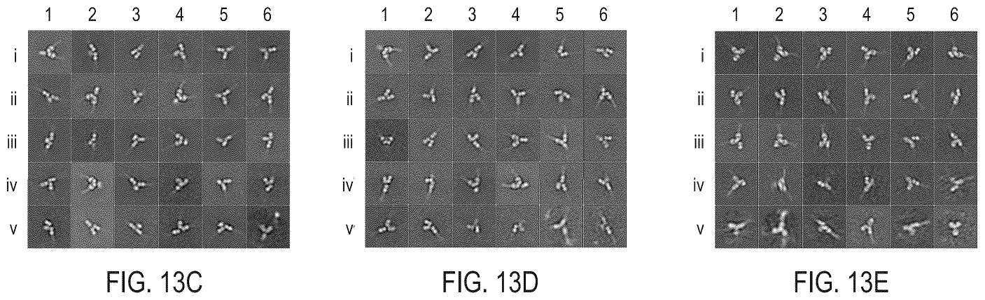

Three IgG-CD40 complexes were evaluated: IgG1-CD40 complex, IgG1-PVA-CD40 complex, and IgG2-CD40 complex. Approximately 35%, 35%, and 11% of particles in the 2D classification runs for IgG1-CD40, IgG1-PVA-CD40, and IgG2-CD40, respectively, had IgG-like features but were not assigned Fab-Fab angles due to ambiguity in Fab arm identification. For the 2D classification runs shown in FIGS. 13C-E, only a small minority of particles (i.e., less than 2%) were classified into ‘junk’ 2D classes lacking IgG-like features.

Fab-Fab angles determined from 2D class averages and particle population percentages are set forth in Table 6.

TABLE 6

| IgG-CD40 | | Fab-Fab | Particle |
|---|---|---|---|
| complex | Representation | angle | population % |
| IgG1-CD40 | FIG. 13C: i1 | 61° | 16 |
| IgG1-CD40 | FIG. 13C: i2 | 210° | 10 |
| IgG1-CD40 | FIG. 13C: i5 | 84° | 5 |
| IgG1-CD40 | FIG. 13C: i6 | 103° | 5 |
| IgG1-CD40 | FIG. 13C: ii1 | 175° | 5 |
| IgG1-CD40 | FIG. 13C: ii2 | 61° | 4 |
| IgG1-CD40 | FIG. 13C: ii4 | 73° | 3 |
| IgG1-CD40 | FIG. 13C: ii5 | 109° | 3 |
| IgG1-CD40 | FIG. 13C: iii1 | 210° | 3 |
| IgG1-CD40 | FIG. 13C: iii3 | 126° | 3 |
| IgG1-CD40 | FIG. 13C: iii4 | 58° | 2 |
| IgG1-CD40 | FIG. 13C: iv2 | 75° | 2 |
| IgG1-CD40 | FIG. 13C: iv3 | 135° | 1 |
| IgG1-CD40 | FIG. 13C: v3 | 178° | 1 |
| IgG1-CD40 | FIG. 13C: iv5 | 103° | 1 |
| IgG1-PVA-CD40 | FIG. 13D: i1 | 65° | 22 |
| IgG1-PVA-CD40 | FIG. 13D: i3 | 177° | 6 |
| IgG1-PVA-CD40 | FIG. 13D: i4 | 164° | 6 |
| IgG1-PVA-CD40 | FIG. 13D: i5 | 53° | 6 |
| IgG1-PVA-CD40 | FIG. 13D: i6 | 207° | 6 |
| IgG1-PVA-CD40 | FIG. 13D: ii3 | 50° | 4 |
| IgG1-PVA-CD40 | FIG. 13D: ii5 | 231° | 3 |
| IgG1-PVA-CD40 | FIG. 13D: ii6 | 69° | 3 |
| IgG1-PVA-CD40 | FIG. 13D: iii4 | 56° | 2 |
| IgG1-PVA-CD40 | FIG. 13D: iii5 | 58° | 2 |
| IgG1-PVA-CD40 | FIG. 13D: iv3 | 61° | 1 |
| IgG1-PVA-CD40 | FIG. 13D: iv4 | 62° | 1 |
| IgG1-PVA-CD40 | FIG. 13D: iv5 | 158° | 1 |
| IgG1-PVA-CD40 | FIG. 13D: iv6 | 138° | 1 |
| IgG2-CD40 | FIG. 13E: i1 | 93° | 17 |
| IgG2-CD40 | FIG. 13E: i2 | 82° | 11 |
| IgG2-CD40 | FIG. 13E: i3 | 94° | 7 |
| IgG2-CD40 | FIG. 13E: i4 | 88° | 7 |
| IgG2-CD40 | FIG. 13E: i5 | 86° | 6 |
| IgG2-CD40 | FIG. 13E: i6 | 87° | 6 |
| IgG2-CD40 | FIG. 13E: ii1 | 86° | 6 |
| IgG2-CD40 | FIG. 13E: ii2 | 218° | 5 |
| IgG2-CD40 | FIG. 13E: ii3 | 105° | 4 |
| IgG2-CD40 | FIG. 13E: ii4 | 155° | 4 |
| IgG2-CD40 | FIG. 13E: ii5 | 81° | 4 |
| IgG2-CD40 | FIG. 13E: ii6 | 219° | 3 |
| IgG2-CD40 | FIG. 13E: iii1 | 93° | 3 |
| IgG2-CD40 | FIG. 13E: iii2 | 89° | 3 |
| IgG2-CD40 | FIG. 13E: iii3 | 111° | 2 |

Without being bound by theory, the IgG1-PVA-Fc domains are believed to result in increased target binding of an antibody preparation by virtue of increasing the percentage of molecules in the antibody preparation with Fab-Fab angles suitable for target binding.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A multispecific antibody comprising a first polypeptide chain comprising a chimeric constant domain comprising, from N-terminus to C-terminus:
(a) a chimeric immunoglobulin hinge comprising:
(i) a human IgG1 upper hinge amino acid sequence EPKSCDKTHT (SEQ ID NO: 24) from positions 216 to 225 (EU numbering)
(ii) a human IgG1 core hinge amino acid sequence CPPC (SEQ ID NO: 25) from positions 226 to 229 (EU numbering);
(iii) a human IgG2 lower hinge amino acid sequence PCPAPPVAGP (SEQ ID NO: 42) from positions 230 to 238 (EU numbering);
(b) a CH2 domain comprising a human IgG1 CH2 domain amino acid sequence from position 239 to 340 (EU numbering) of human IgG1, optionally comprising one or more CH2 domain amino acid substitutions wherein if the CH2 has one or more of amino acid substitutions H268Q, K274Q, Y296F, A327G, A330S, and P331S (EU numbering), they do not occur simultaneously; and
(c) a CH3 domain comprising a human IgG1 CH3 domain amino acid sequence from position 341 to 447 (EU numbering) of human IgG1, optionally comprising one or more CH3 domain amino acid substitutions selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing.

2. A multispecific antibody comprising a first polypeptide chain associated with a second polypeptide chain, wherein:
(a) the first polypeptide chain comprises: a first chimeric constant domain comprising an amino acid sequence that has at least 95% identity to SEQ ID NO:1, provided that chimeric constant domain has the sequence P-V-A-absent at amino acids 233 to 236 as defined by EU numbering; and
(b) the second polypeptide chain comprises a second chimeric constant domain comprising an amino acid sequence that has at least 95% identity to SEQ ID NO:1, provided that chimeric constant domain has the sequence P-V-A-absent at amino acids 233 to 236 as defined by EU numbering.

3. A multispecific antibody comprising a first polypeptide chain associated with a second polypeptide chain, wherein:
(a) the first polypeptide chain comprises a first chimeric constant domain comprising, from N-terminus to C-terminus:
(i) a chimeric immunoglobulin hinge comprising:
(1) a human IgG1 upper hinge amino acid sequence EPKSCDKTHT from positions 216 to 225 (EU numbering)
(2) a human IgG1 core hinge amino acid sequence CPPC from positions 226 to 229 (EU numbering);
(3) a human IgG2 lower hinge amino acid sequence PCPAPPVAGP from positions 230 to 238 (EU numbering);
(ii) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A327G, A330S, and P331S (EU numbering), they do not occur simultaneously; and
(iii) a CH3 domain comprising a human IgG1 CH3 domain amino acid sequence from position 341 to 447 (EU numbering) of human IgG1, optionally comprising one or more CH3 domain amino acid substitutions selected from knob substitutions (e.g., T366W), hole substitutions (e.g., T366S, L368A, Y407V), star mutations (e.g., H435R, Y436F), mutations that introduce a disulfide bridge (e.g., S354C or E357C), or a combination of two or more of the foregoing; and
(b) the second polypeptide chain comprises a second chimeric constant domain comprising, from N-terminus to C-terminus:
(i) a chimeric immunoglobulin hinge comprising:
(1) a human IgG1 upper hinge amino acid sequence EPKSCDKTHT from positions 216 to 225 (EU numbering)
(2) a human IgG1 core hinge amino acid sequence CPPC from positions 226 to 229 (EU numbering);
(3) a human IgG2 lower hinge amino acid sequence PCPAPPVAGP from positions 230 to 238 (EU numbering);
(ii) a CH2 domain comprising a human IgG1 CH2 domain amino acid sequence from position 239 to 340 (EU numbering) of human IgG1, optionally comprising one or more CH2 domain amino acid substitutions and optionally wherein if the CH2 has one or more of amino acid substitutions H268Q, K274Q, Y296F, A327G, A33S, and P331S (EU numbering), they do not occur simultaneously; and
(iii) a CH3 domain comprising a human IgG1 CH3 domain amino acid sequence from position 341 to 447 (EU numbering) of human IgG1, optionally comprising one or more CH3 domain amino acid substitutions, optionally wherein the substitutions are selected from knob substitutions (e.g., T366W)), hole substitutions (e.g., T366S, L368A, Y407V), star mutations (e.g., H435R, Y436F), mutations that introduce a disulfide bridge (e.g., S354C or E357C), or a combination of two or more of the foregoing.

4. The multispecific antibody any one of claims 1 to 3, wherein the first chimeric constant domain comprises one or more amino acid substitutions that promote heterodimerization with the second polypeptide chain.

5. The multispecific antibody of claim 4, wherein the one or more amino acid substitutions that promote heterodimerization with the second polypeptide chain are one or more of S354C and T366W (EU numbering).

6. The multispecific antibody of claim 4, wherein the one or more amino acid substitutions that promote heterodimerization with the second polypeptide chain are one or more of Y349C, T366S, L368A and Y407V (EU numbering).

7. The multispecific antibody of any one of claims 1 to 6, wherein the first chimeric constant domain comprises an amino acid sequence having at least 97% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

8. The multispecific antibody of any one of claims 1 to 7, wherein the first chimeric constant domain comprises an amino acid sequence having at least 98% sequence identity to any one of SEQ ID NO:3, SEQ ID NO: 15, SEQ ID NO:16, SEQ ID NO:17, SEQ ID NO:18, SEQ ID NO:19, SEQ ID NO:20, SEQ ID NO:21 or SEQ ID NO:22.

9. The multispecific antibody of any one of claims 1 to 3, wherein the first chimeric constant domain comprises an amino acid sequence having at least 99% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

10. The multispecific antibody of any one of claims 1 to 9, wherein the first chimeric constant domain comprises the amino acid sequence of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

11. The multispecific antibody of any one of claims 1 to 10, wherein the second chimeric constant domain comprises one or more amino acid substitutions that promote heterodimerization with the first polypeptide chain.

12. The multispecific antibody of claim 11, wherein the one or more amino acid substitutions that promote heterodimerization with the first polypeptide chain are one or more of S354C and T366W (EU numbering).

13. The multispecific antibody of claim 11, wherein the one or more amino acid substitutions that promote heterodimerization with the first polypeptide chain are one or more of Y3490, T366S, L368A and Y407V (EU numbering).

14. The multispecific antibody of any one of claims 1 to 13, wherein the second chimeric constant domain comprises an amino acid sequence having at least 97% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

15. The multispecific antibody of any one of claims 1 to 14, wherein the second chimeric constant domain comprises an amino acid sequence having at least 98% sequence identity to any one of SEQ ID NO:3, SEQ ID NO:15, SEQ ID NO:16, SEQ ID NO:17, SEQ ID NO:13, SEQ ID NO:19, SEQ ID NO:20, SEQ ID NO:21 or SEQ ID NO:22.

16. The multispecific antibody of any one of claims 1 to 15, wherein the second chimeric constant domain comprises an amino acid sequence having at least 99% sequence identity to any one of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

17. The multispecific antibody of any one of claims 1 to 16, wherein the second chimeric constant domain comprises the amino acid sequence of SEQ ID NO:3, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

18. The multispecific antibody of any one of claims 1 to 16, wherein the first chimeric constant domain and the second chimeric constant domain are identical.

19. The multispecific antibody of claim 18, wherein the multispecific antibody is a homodimer.

20. The multispecific antibody of any one of claims 1 to 16, wherein the first chimeric constant domain and the second chimeric constant domain are different.

21. The multispecific antibody of claim 20, wherein the first constant domain comprises the amino acid sequence of SEQ ID NO:15 and the second constant domain comprises the amino acid sequence of SEQ ID NO:16.

22. The multispecific antibody of claim 20, The multispecific antibody of any one of claims 1 to 17, wherein the first constant domain comprises the amino acid sequence of SEQ ID NO:17 and the second constant domain comprises the amino acid sequence of SEQ ID NO:16.

23. The multispecific antibody of claim 20, wherein the first constant domain comprises the amino acid sequence of SEQ ID NO:15 and the second constant domain comprises the amino acid sequence of SEQ ID NO:18.

24. The multispecific antibody of claim 20, wherein the first constant domain comprises the amino acid sequence of SEQ ID NO:17 and the second constant domain comprises the amino acid sequence of SEQ ID NO:18.

25. The multispecific antibody of claim 20, wherein the first constant domain comprises the amino acid sequence of SEQ ID NO:19 and the second constant domain comprises the amino acid sequence of SEQ ID NO:20.

26. The multispecific antibody of claim 20, wherein the first constant domain comprises the amino acid sequence of SEQ ID NO:19 and the second constant domain comprises the amino acid sequence of SEQ ID NO:22.

27. The multispecific antibody of claim 20, wherein the first constant domain comprises the amino acid sequence of SEQ ID NO:21 and the second constant domain comprises the amino acid sequence of SEQ ID NO:20.

28. The multispecific antibody of claim 20, wherein the first constant domain comprises the amino acid sequence of SEQ ID NO:21 and the second constant domain comprises the amino acid sequence of SEQ ID NO:22.

29. The multispecific antibody of any one of claims 20 to 28, wherein the multispecific antibody is a heterodimer.

30. The multispecific antibody of any one of claims 1 to 29, further comprising a first target binding domain and a second target binding domain.

31. The multispecific antibody of claim 30, wherein the first target binding domain binds to a first target molecule and the second target binding domain binds to a second target molecule.

32. The multispecific antibody of claim 30, wherein the first target binding domain and the second target binding domain bind to the same target molecule.

33. The multispecific antibody of any one of claims 30 to 32, further comprising a third target binding domain binds to a different target molecule than the first target binding domain and/or the second target binding domain.

34. The multispecific antibody of any one of claims 1 to 33, wherein the multispecific antibody is a bispecific antibody.

35. The multispecific antibody of any one of claims 1 to 33, wherein the multispecific antibody is a trispecific antibody.

36. The multispecific antibody of any one of claims 1 to 35, wherein the multispecific antibody is a trivalent antibody.

37. The multispecific antibody of any one of claims 1 to 35, wherein the multispecific antibody is a tetravalent antibody.

38. The multispecific antibody of any one of claims 1 to 37, wherein the multispecific antibody exhibits increased expression of at least 5%, 10%, 15%, 20%, 25%, 30%, 40%, 50%, 60%, 70%, 80%, 90%, or 100% in an expression system compared to an antibody comprising a wild type IgG1 constant domain comprising IgG1 hinge, IgG1 CH2, and IgG1 CH3.

39. The multispecific antibody of any one of claims 1 to 38, wherein the multispecific antibody exhibits at least 5-fold, at least 10-fold, at least 50-fold, at least 100-fold, at least 500-fold, at least 1,000-fold, at least 5,000-fold, or at least 10,000-fold reduced binding to a human Fc receptor (FcR) than a polypeptide comprising a wild type IgG1 constant domain comprising IgG1 hinge, IgG1 CH2, and IgG1 CH3.

40. The multispecific antibody of claim 39, wherein the FcR is FcRγ1, FcRγ2A, FcRγ2B, FcRγ3A, or FcRγ3B.

41. A composition comprising the multispecific antibody of any one of claims 1 to 40.

42. The composition of claim 41, wherein the composition is a pharmaceutical composition and further comprises one or more excipients and/or pharmaceutically acceptable carriers.

43. A nucleic acid molecule or a plurality of nucleic acid molecules encoding the multispecific antibody of any one of claims 1 to 40.

44. A host cell engineered to express the multispecific antibody of any one of claims 1 to 40.

45. A method of producing the multispecific antibody of any one of claims 1 to 40, comprising culturing the host cell of claim 44 and optionally recovering and/or purifying the expressed protein.

46. A method for increasing production of a multispecific antibody, comprising expressing the multispecific antibody as a multispecific antibody a defined in any one of claims 1 to 40.

47. The method of claim 46, which comprises culturing the host cell of claim 44 such that the protein is expressed and recovering the expressed protein.

48. The method of claim 46 or 47, wherein the method increases expression by at least 10%, at least 15%, at least 20%, at least 30%, at least 40%, or at least 50% relative to expression of a variant protein comprising a control pair of constant domains each comprising the sequence E-L-L-G at amino acids 233 to 236 as defined by EU numbering.

49. A population of multispecific antibodies according to any one of claims 1 to 40, optionally comprising at least 10,000 multispecific antibodies.

50. The population of multispecific antibodies of claim 49, which is characterized by greater activity as compared to a population of proteins comprising a control pair of constant domains each comprising the sequence E-L-L-G at amino acids 233 to 236 as defined by EU numbering.


SEQUENCE LISTING (59 sequences)

SEQ ID NO: 1 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 4 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 7 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 5 aa, synthetic construct
GGGGS

SEQ ID NO: 12 - protein, 7 aa, synthetic construct
GGGGSGG

SEQ ID NO: 13 - protein, 15 aa, synthetic construct
GGGGSGGGGSGGGGS

SEQ ID NO: 14 - protein, 30 aa, synthetic construct
GGGGSGGGGSGGGGSGGGGSGGGGSGGGGS

SEQ ID NO: 15 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 18 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 19 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 22 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 23 - protein, 4 aa, synthetic construct
ELLG

SEQ ID NO: 24 - protein, 10 aa, Homo sapiens
EPKSCDKTHT

SEQ ID NO: 25 - protein, 4 aa, Homo sapiens
CPPC

SEQ ID NO: 26 - protein, 7 aa, Homo sapiens
PAPELLG

SEQ ID NO: 27 - protein, 104 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 28 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 6 aa, synthetic construct
PAPPVA

SEQ ID NO: 30 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 31 - protein, 4 aa, synthetic construct
DKKV

SEQ ID NO: 32 - protein, 4 aa, synthetic construct
DKRV

SEQ ID NO: 33 - protein, 11 aa, synthetic construct
  VARIANT 1..10: note = This region may encompass 1-10 residues
GGGGGGGGGGS

SEQ ID NO: 34 - protein, 11 aa, synthetic construct
  VARIANT 2..11: note = This region may encompass 1-10 residues
SGGGGGGGGGG

SEQ ID NO: 35 - protein, 5 aa, synthetic construct
GGGGS

SEQ ID NO: 36 - protein, 4 aa, synthetic construct
GGGG

SEQ ID NO: 37 - protein, 5 aa, synthetic construct
GGGGG

SEQ ID NO: 38 - protein, 6 aa, synthetic construct
GGGGGG

SEQ ID NO: 39 - protein, 7 aa, synthetic construct
GGGGGGG

SEQ ID NO: 40 - protein, 8 aa, synthetic construct
GGGGGGGG

SEQ ID NO: 41 - protein, 9 aa, synthetic construct
GGGGGGGGG

SEQ ID NO: 42 - protein, 10 aa, Homo sapiens
PCPAPPVAGP

SEQ ID NO: 43 - protein, 12 aa, Homo sapiens
EPKSCDKTHTCP

SEQ ID NO: 44 - protein, 8 aa, Homo sapiens
PCPAPPVA

SEQ ID NO: 45 - protein, 5 aa, Homo sapiens
DISVE

SEQ ID NO: 46 - protein, 5 aa, synthetic construct
DIAVE

SEQ ID NO: 47 - protein, 6 aa, synthetic construct
HHHHHH

SEQ ID NO: 48 - protein, 330 aa, Homo sapiens [sequence omitted]

SEQ ID NO: 49 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 50 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 22 aa, synthetic construct
EPKSCDKTHTCPPCPAPPVAGP

SEQ ID NO: 52 - protein, 23 aa, synthetic construct
EPKSCDKTHTCPPCPAPELLGGP

SEQ ID NO: 53 - protein, 19 aa, synthetic construct
ERKCCVECPPCPAPPVAGP

SEQ ID NO: 54 - protein, 20 aa, synthetic construct
ESKYGPPCPPCPAPEFLGGP

SEQ ID NO: 55 - protein, 19 aa, synthetic construct
ESKYGPPCPPCPAPPVAGP

SEQ ID NO: 56 - protein, 10 aa, synthetic construct
EPKSCDKTHT

SEQ ID NO: 57 - protein, 4 aa, synthetic construct
CPPC

SEQ ID NO: 58 - protein, 104 aa, synthetic construct; identical to SEQ ID NO: 27

SEQ ID NO: 59 - protein, 107 aa, synthetic construct; identical to SEQ ID NO: 28